(19) United States
(12) Patent Application Publication
(10) Pub. No.: US 2025/0011352 A1
(43) Pub. Date: Jan. 9, 2025

(54) SOLID FORMS

(71) Applicant: Gilead Sciences, Inc., Foster City, CA (US)

(72) Inventors: Ekaterina V. Albert, West Lafayette, IN (US); Yisoo M. Hwang, West Lafayette, IN (US); Bing Shi, Redwood City, CA (US)

(21) Appl. No.: 18/678,355

(22) Filed: May 30, 2024

Related U.S. Application Data
(60) Provisional application No. 63/505,226, filed on May 31, 2023.

(51) Int. Cl. C07F 9/6558; A61K 31/675; A61K 45/06; C07C 55/06; C07C 59/255; C07C 65/05; C07C 65/21

(52) U.S. Cl. CPC C07F 9/65583; A61K 31/675; A61K 45/06; C07C 55/06; C07C 59/255; C07C 65/05; C07C 65/21

(57) ABSTRACT

The present disclosure relates to solid forms of compounds and pharmaceutical compositions thereof, which are useful in the treatment and prevention of a Retroviridae viral infection including an infection caused by the HIV virus

### CROSS REFERENCE TO RELATED APPLICATIONS

This application claims the benefit of U.S. Provisional Application No. 63/505,226, filed on May 31, 2023, the entire contents of which is hereby incorporated by reference in its entirety.

### TECHNICAL FIELD

This disclosure relates generally to solid forms of a compound useful in the prevention or treatment of a Retroviridae viral infection, including an infection caused by the human immunodeficiency virus (HIV). This disclosure also relates to methods of making said compounds and intermediates in the preparation of said compounds.

### BACKGROUND

Positive-single stranded RNA viruses comprising the Retroviridae family include those of the subfamily Orthoretrovirinae and genera Alpharetrovirus, Betaretrovirus, Gammaretrovirus, Deltaretrovirus, Epsilonretrovirus, Lentivirus, and Spumavirus which cause many human and animal diseases. Among the Lentivirus, HIV-1 infection in humans leads to depletion of T helper cells and immune dysfunction, producing immunodeficiency and vulnerability to opportunistic infections. Treating HIV-1 infections with highly active antiretroviral therapies (HAART) has proven to be effective at reducing viral load and significantly delaying disease progression (Hammer, S. M., et al.; JAMA 2008, 300:555-570). However, these treatments could lead to the emergence of HIV strains that are resistant to current therapies (Taiwo, B., International Journal of Infectious Diseases 2009, 13:552-559; Smith, R. J., et al., Science 2010, 327:697-701). Therefore, there is a pressing need to discover new antiretroviral agents that are active against emerging drug-resistant HIV variants.

Also of interest in the area of HIV therapies and treatments is providing regimens to patients with improved pharmacokinetic properties, including, for example, increased potency, long-acting pharmacokinetics, low solubility, low clearance, and/or other properties. While current regimens for treating HIV have progressed enough that patients no longer have to take multiple pills multiple times a day, patients today still are required to take a pill every day for the foreseeable span of their life. Thus, it would be beneficial to have HIV therapies that require patients take medication less than once a day (e.g. once every couple of days, once a week, once every other week, once a month, and so forth) or take a smaller effective dose of the medication(s) on a daily, weekly, monthly, or longer basis.

### SUMMARY

The present disclosure provides, inter alia, a crystalline form of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid.

The present disclosure further provides a solvate form of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid.

The present disclosure further provides a co-crystal of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid.

The present disclosure further provides a pharmaceutical composition comprising a crystalline form, solvate form, or co-crystal disclosed herein, and at least one pharmaceutically acceptable excipient.

The present disclosure further provides a method of treating or preventing a human immunodeficiency virus (HIV) infection comprising administering a therapeutically effective amount of a crystalline form, solvate form, or co-crystal disclosed herein, to a subject in need thereof.

The present disclosure further provides a crystalline form, solvate form, or co-crystal disclosed herein, for use in therapy.

The present disclosure further provides a crystalline form, solvate form, or co-crystal disclosed herein, for use in a method of treating or preventing a human immunodeficiency virus (HIV) infection, comprising administering a therapeutically effective amount of the crystalline or solvate form to a subject in need thereof.

### DESCRIPTION OF DRAWINGS

FIG. 1 shows the structure of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid (i.e., Compound 1) and its restricted rotational axes.

FIG. 2 shows a representative XRPD pattern of Compound 1, acetonitrile (MeCN) solvate.

FIG. 3 shows representative XRPD patterns of the solvate precursors of Compound 1, crystalline Form I (from top to bottom): MeCN solvate, MIBK solvate 1, MIBK solvate 2, toluene solvate 2, butyronitrile solvate, nitromethane solvate, ethyl ether solvate, anisole solvate 2, and xylene solvate.

FIG. 4 shows a representative XRPD pattern of Compound 1, crystalline Form I.

FIG. 5 shows a representative DSC thermogram of Compound 1, crystalline Form I.

FIG. 6 shows a representative TGA thermogram of Compound 1, crystalline Form I.

FIG. 7 shows a representative DVS analysis of Compound 1, crystalline Form I.

FIG. 8 shows representative XRPD patterns of the solvate precursors of Compound 1, crystalline Form II (from top to bottom): DCM solvate 2, BuOAc solvate 2, MEK solvate, EtOAc solvate 2, chloroform solvate 2, acetophenone solvate, CPME solvate, and DCE solvate.

FIG. 9 shows an overlay of the XRPD patterns of Compound 1, crystalline Form II from simulation (top) and experiment (bottom).

FIG. 10 shows an overly of the XRPD patterns of Compound 1, crystalline Form II obtained after drying BuOAc solvate 2 (top) and DCM solvate 2 (bottom).

FIG. 11 shows a representative XRPD pattern of Compound 1, crystalline Form II.

FIG. 12 shows a representative DSC thermogram of Compound 1, crystalline Form II.

FIG. 13 shows a representative TGA thermogram of Compound 1, crystalline Form II.

FIG. 14 shows a representative DVS analysis of Compound 1, crystalline Form II.

FIG. 15 shows representative XRPD patterns of the solvate precursors of Compound 1, crystalline Form III: IPAc solvate 2 (top) and MIBK solvate 3 (bottom).

FIG. 16 shows a representative XRPD pattern of Compound 1, crystalline Form III.

FIG. 17 shows a representative DSC thermogram of Compound 1, crystalline Form III.

FIG. 18 shows a representative TGA thermogram of Compound 1, crystalline Form III.

FIG. 19 shows a representative DVS analysis of Compound 1, crystalline Form III.

FIG. 20 shows representative XRPD patterns of the solvate precursors of Compound 1, Material A (from top to bottom): EtOAc solvate 1, MeOAc solvate, IPAc solvate 1, BuOAc solvate 1, DCM solvate 1, chloroform solvate 1, toluene solvate 1, cyclohexane solvate, and anisole solvate 1.

FIG. 21 shows a representative XRPD pattern of Compound 1, Material A.

FIG. 22 shows a representative reversible heat thermogram from MDSC analysis of Compound 1, Material A.

FIG. 23 shows a representative TGA thermogram of Compound 1, Material A.

FIG. 24 shows a representative DVS analysis of Compound 1, Material A.

FIG. 25 shows an overlay of the spectra of Compound 1, Material A (top), and amorphous Compound 1 (bottom) from 19F-NMR analysis.

FIG. 26 shows a representative XRPD pattern of amorphous Compound 1.

FIG. 27 shows a representative reversible heat thermogram from MDSC analysis of amorphous Compound 1.

FIG. 28 shows a representative TGA thermogram of amorphous Compound 1.

FIG. 29 shows a representative XRPD pattern of Compound 1, maleate co-crystal.

FIG. 30 shows a representative DSC thermogram of Compound 1, maleate co-crystal.

FIG. 31 shows a representative TGA thermogram of Compound 1, maleate co-crystal.

FIG. 32 shows a representative DVS analysis of Compound 1, maleate co-crystal.

FIG. 33 shows a representative XRPD pattern of Compound 1, succinate co-crystal.

FIG. 34 shows a representative DSC thermogram of Compound 1, succinate co-crystal.

FIG. 35 shows a representative TGA thermogram of Compound 1, succinate co-crystal.

FIG. 36 shows a representative DVS analysis of Compound 1, succinate co-crystal.

FIG. 37 shows representative XRPD patterns of various solvate precursors of Compound 2, crystalline Form I (from top to bottom): IPA solvate, MTBE solvate, and CPME solvate.

FIG. 38 shows a representative XRPD pattern of Compound 2, crystalline Form I.

FIG. 39 shows a representative DSC thermogram of Compound 2, crystalline Form I.

FIG. 40 shows a representative TGA thermogram of Compound 2, crystalline Form I.

FIG. 41 shows a representative DVS analysis of Compound 2, crystalline Form I.

FIG. 42 shows representative XRPD patterns of acetone solvate of Compound 2 (bottom), and Compound 2, crystalline Form II (top, obtained after drying the acetone solvate at 40° C. under vacuum).

FIG. 43 shows the crystal structure of Compound 2, acetone solvate (hydrogen atoms were not shown and solvent molecules were not modeled).

FIG. 44 shows a representative XRPD pattern of Compound 2, crystalline Form II.

FIG. 45 shows a representative DSC thermogram of Compound 2, crystalline Form II.

FIG. 46 shows a representative TGA thermogram of Compound 2, crystalline Form II.

FIG. 47 shows a representative XRPD pattern of Compound 2, crystalline Form III.

FIG. 48 shows a representative DSC thermogram of Compound 2, crystalline Form III.

FIG. 49 shows a representative TGA thermogram of Compound 2, crystalline Form III.

FIG. 50 shows a representative XRPD pattern of amorphous Compound 2.

FIG. 51 shows a representative DSC thermogram of amorphous Compound 2.

FIG. 52 shows the crystal structure of Compound 2, acetonitrile (MeCN) solvate (hydrogen atoms were not shown and solvent molecules were not modeled).

FIG. 53 shows the crystal structure of Compound 1, ethyl acetate (EtOAc) solvate 1 (hydrogen atoms were not shown and solvent molecules were not modeled).

FIG. 54 shows the crystal structure of Compound 1, crystalline Form II, recrystallized from dichloromethane (DCM) (hydrogen atoms were not shown and solvent molecules were not modeled).

FIG. 55 shows the crystal structure of Compound 1, crystalline Form II, recrystallized from butyl acetate (BuOAc) (hydrogen atoms were not shown and solvent molecules were not modeled).

FIG. 56 shows the crystal structure of Compound 1, butyl acetate (BuOAc) solvate 2 (hydrogen atoms were not shown and solvent molecules were not modeled).

FIG. 57 shows a representative XRPD pattern of Compound 1, oxalate co-crystal.

FIG. 58 shows a representative DSC thermogram of Compound 1, oxalate co-crystal.

FIG. 59 shows a representative XRPD pattern of Compound 1, gentisate co-crystal.

FIG. 60 shows a representative DSC thermogram of Compound 1, gentisate co-crystal.

FIG. 61 shows a representative XRPD pattern of Compound 1, L-tartrate co-crystal.

FIG. 62 shows a representative DSC thermogram of Compound 1, L-tartrate co-crystal.

FIG. 63 shows a representative XRPD pattern of Compound 1, vanillate co-crystal.

FIG. 64 shows a representative DSC thermogram of Compound 1, vanillate co-crystal.

### DETAILED DESCRIPTION

The present invention relates to new solid forms, e.g., crystalline forms, solvate forms, and/or co-crystals of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid (i.e., Compound 1, structure shown below; see e.g. U.S. application Ser. No. 18/061,375, the disclosure of which is incorporated herein by reference in its entirety).

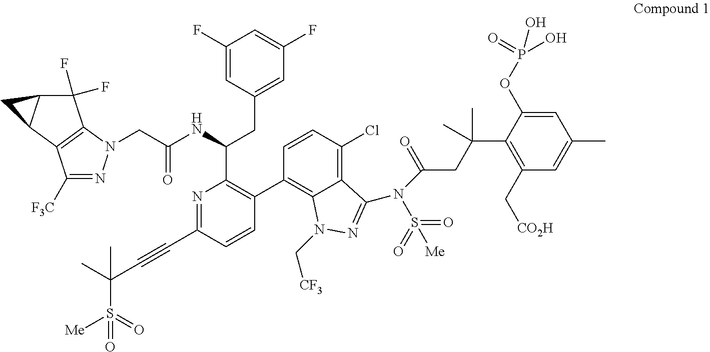 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

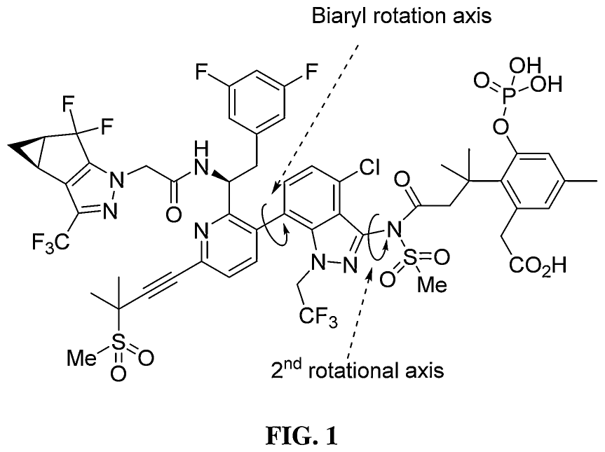

Compound 1 has two restricted rotational axes, resulting in 4 atropisomers (see FIG. 1) that may be detected by ¹⁹F-NMR. In deuterated DMSO at 25° C., the half-life of conversion from the major to the minor atropisomer for the biaryl rotation is about 71.6 hours with equilibrium ratio at about 3:1, and the half-life of interconversion at the 2^{nd} rotational axis is about 7 minutes with equilibrium ratio at about 4:3.

Compound 1 converts to lenacapavir (i.e., N—((S)-1-(3-(4-chloro-3-(methylsulfonamido)-1-(2,2,2-trifluoroethyl)-1H-indazol-7-yl)-6-(3-methyl-3-(methylsulfonyl) but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide), an HIV capsid inhibitor that is in development as a long-acting treatment for HIV, in the gastrointestinal tract when administered to a subject (e.g., a human patient).

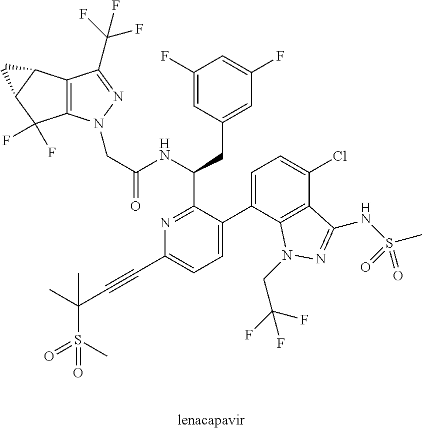 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Synthesis and characterization of lenacapavir, and salts thereof, are described, for example, in US20180051005 and US20190300505, the contents of each of which are hereby incorporated by reference in their entireties. Various forms and/or uses of the compounds of lenacapavir are disclosed, for example, in US20190083478, US20190084963, US 20200038389A1, and US20210188815, the contents of each of which are hereby incorporated by reference in their entireties.

The present application further relates to new solid forms, e.g., crystalline forms, of tert-butyl 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-3-((di-tert-butoxyphosphoryl)oxy)-5-methylphenyl)acetate (i.e., Compound 2, structure shown below).

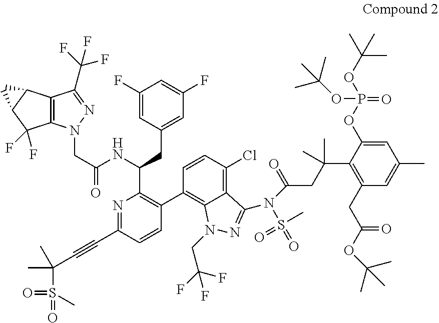 [SMILES: Cc1cc(CC(=O)OC(C)(C)C)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(OC(C)(C)C)OC(C)(C)C)c1]

Compound 2 is an intermediate in the synthesis of Compound 1 (see e.g., U.S. application Ser. No. 18/061,375, the disclosure which is incorporated herein by reference in its entirety). Similar to Compound 1, Compound 2 has two restricted rotational axes, resulting in 4 atropisomers.

### Compound 1, Crystalline Forms

In some embodiments, the present application provides a crystalline form of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid (i.e., Compound 1). In some embodiments, the crystalline form of Compound 1 provided herein is selected from crystalline Form I, crystalline Form II, and crystalline Form III.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. In some embodiments, the crystalline form provided herein may be substantially anhydrous.

In some embodiments, the crystalline form provided herein is Compound 1, crystalline Form I.

In some embodiments, the Compound 1, crystalline Form I has at least one XRPD peak, in terms of 2-theta±0.2°, selected from 6.2°, 6.6°, 8.7°, 10.5°, 12.4°, 12.6°, 13.8°, 23.1°, and 25.7°.

In some embodiments, the Compound 1, crystalline Form I has at least two XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the Compound 1, crystalline Form I has at least three XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the Compound 1, crystalline Form I has at least four XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the Compound 1, crystalline Form I has at least five XRPD peaks, in terms of 2-theta±0.2°, selected from 6.2°, 6.6°, 8.7°, 10.5°, 12.4°, 12.6°, 13.8°, 23.1°, and 25.7°.

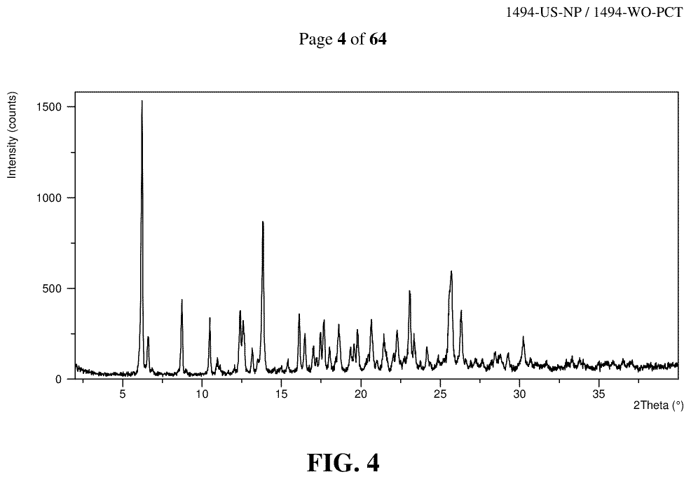

In some embodiments, the Compound 1, crystalline Form I is characterized by an XRPD pattern substantially as shown in FIG. 4.

In some embodiments, the Compound 1, crystalline Form I is characterized by a DSC thermogram having a melting onset at about 202° C.

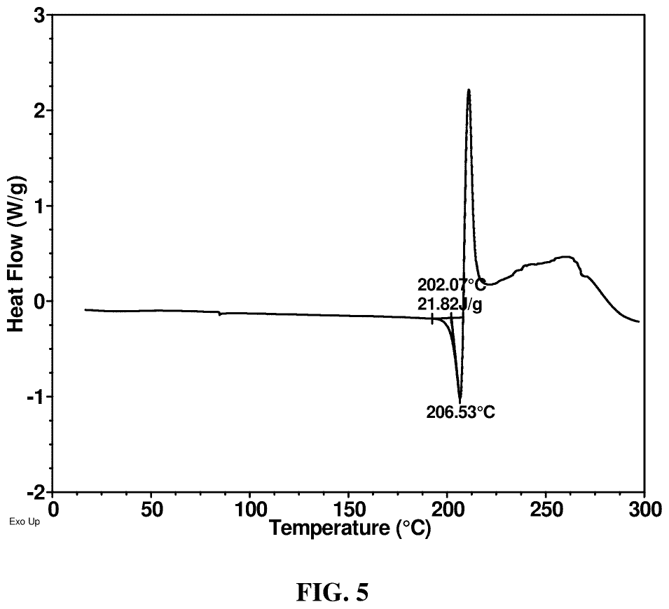

In some embodiments, the Compound 1, crystalline Form I is characterized by a DSC thermogram substantially as shown in FIG. 5.

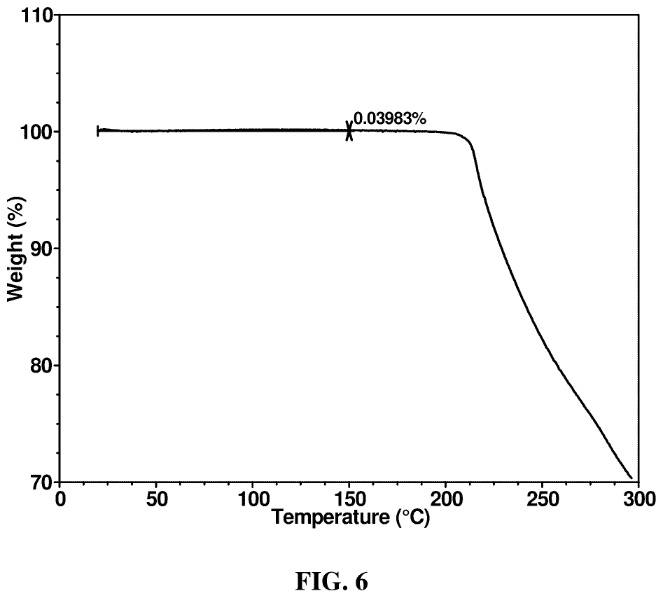

In some embodiments, the Compound 1, crystalline Form I is characterized by a TGA thermogram substantially as shown in FIG. 6.

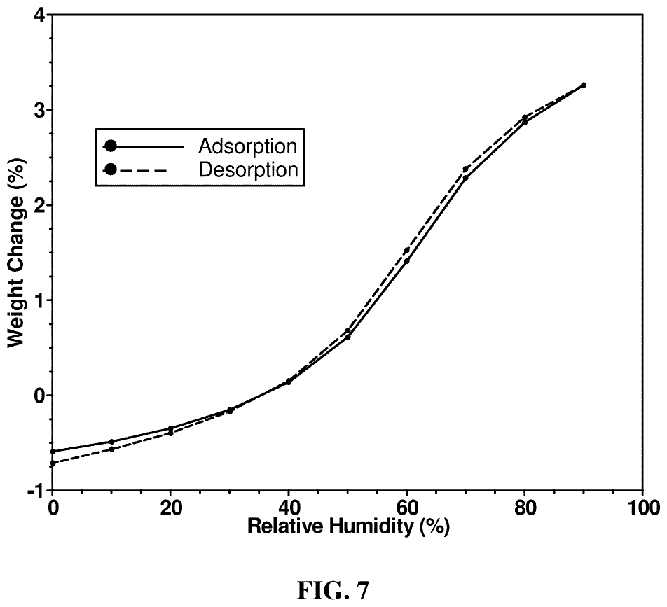

In some embodiments, the Compound 1, crystalline Form I is characterized by a DVS analysis substantially as shown in FIG. 7.

In some embodiments, the crystalline form provided herein is Compound 1, crystalline Form II.

In some embodiments, the Compound 1, crystalline Form II has at least one XRPD peak, in terms of 2-theta±0.2°, selected from 6.5°, 8.9°, 11.4°, 14.3°, 15.7°, 17.2°, 20.0°, 20.8°, and 22.7°.

In some embodiments, the Compound 1, crystalline Form II has at least two XRPD peaks, in terms of 2-theta±0.2°, selected from 6.5°, 8.9°, 11.4°, 14.3°, 15.7°, 17.2°, 20.0°, 20.8°, and 22.7°.

In some embodiments, the Compound 1, crystalline Form II has at least three XRPD peaks, in terms of 2-theta±0.2°, selected from 6.5°, 8.9°, 11.4°, 14.3°, 15.7°, 17.2°, 20.0°, 20.8°, and 22.7°.

In some embodiments, the Compound 1, crystalline Form II has at least four XRPD peaks, in terms of 2-theta±0.2°, selected from 6.5°, 8.9°, 11.4°, 14.3°, 15.7°, 17.2°, 20.0°, 20.8°, and 22.7°.

In some embodiments, the Compound 1, crystalline Form II has at least five XRPD peaks, in terms of 2-theta±0.2°, selected from 6.5°, 8.9°, 11.4°, 14.3°, 15.7°, 17.2°, 20.0°, 20.8°, and 22.7°.

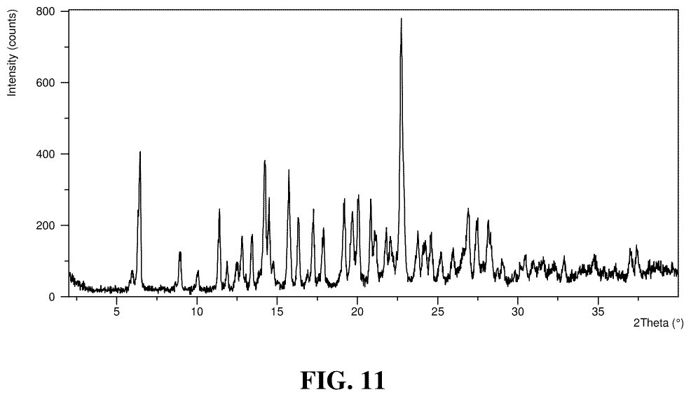

In some embodiments, the Compound 1, crystalline Form II is characterized by an XRPD pattern substantially as shown in FIG. 11.

In some embodiments, the Compound 1, crystalline Form II is characterized by a DSC thermogram having a melting onset at about 201° C.

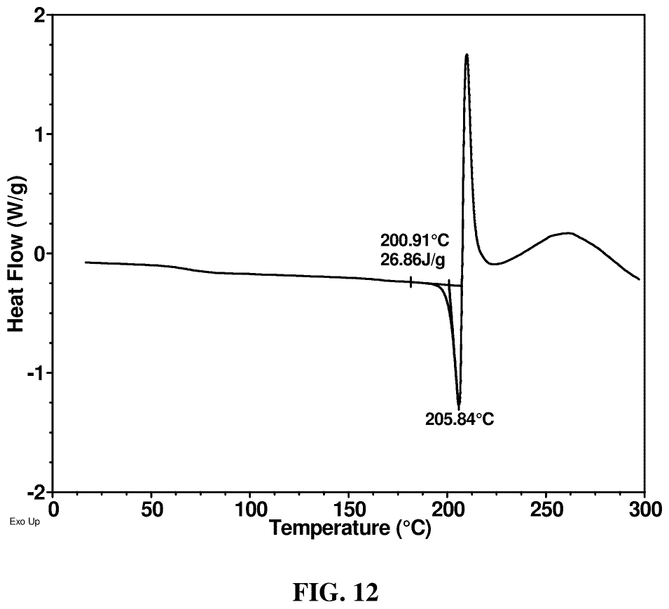

In some embodiments, the Compound 1, crystalline Form II is characterized by a DSC thermogram substantially as shown in FIG. 12.

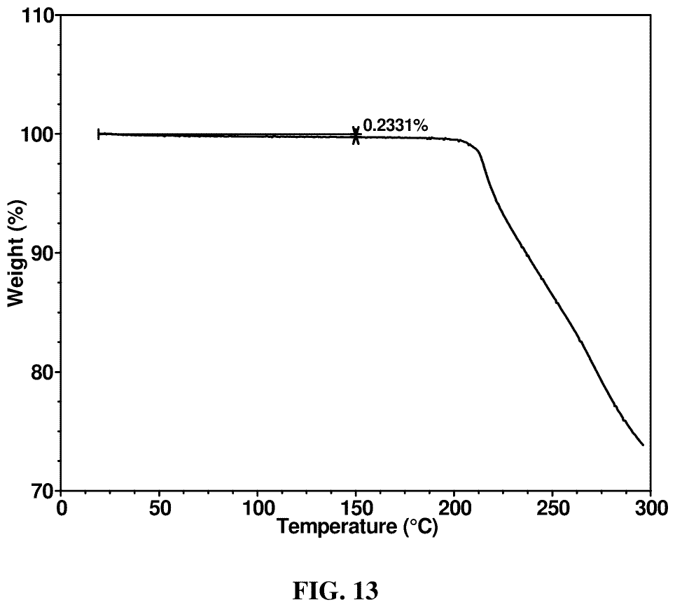

In some embodiments, the Compound 1, crystalline Form II is characterized by a TGA thermogram substantially as shown in FIG. 13.

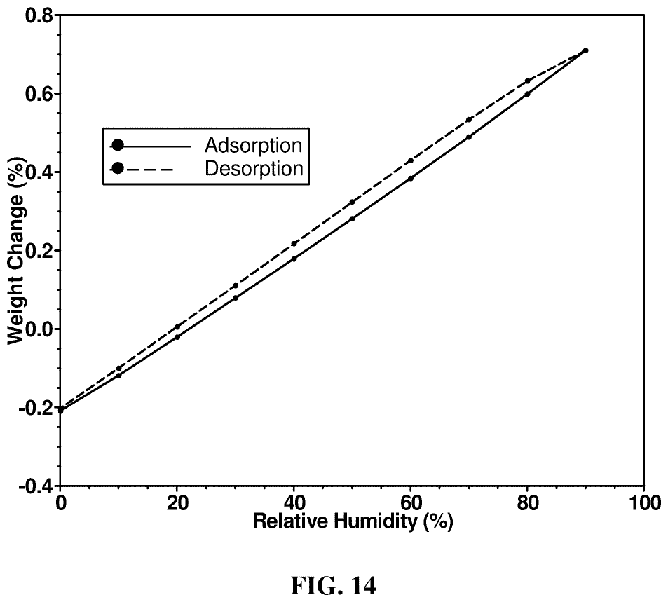

In some embodiments, the Compound 1, crystalline Form II is characterized by a DVS analysis substantially as shown in FIG. 14.

In some embodiments, the crystalline form provided herein is Compound 1, crystalline Form III.

In some embodiments, the Compound 1, crystalline Form III has at least one XRPD peak, in terms of 2-theta±0.2°, selected from 6.5°, 11.8°, 12.8°, 17.4°, 18.7°, 19.2°, 21.5°, 21.9°, and 24.5°.

In some embodiments, the Compound 1, crystalline Form III has at least two XRPD peaks, in terms of 2-theta±0.2°, selected from 6.5°, 11.8°, 12.8°, 17.4°, 18.7°, 19.2°, 21.5°, 21.9°, and 24.5°.

In some embodiments, the Compound 1, crystalline Form III has at least three XRPD peaks, in terms of 2-theta±0.2°, selected from 6.5°, 11.8°, 12.8°, 17.4°, 18.7°, 19.2°, 21.5°, 21.9°, and 24.5°.

In some embodiments, the Compound 1, crystalline Form III has at least four XRPD peaks, in terms of 2-theta±0.2°, selected from 6.5°, 11.8°, 12.8°, 17.4°, 18.7°, 19.2°, 21.5°, 21.9°, and 24.5°.

In some embodiments, the Compound 1, crystalline Form III has at least five XRPD peaks, in terms of 2-theta±0.2°, selected from 6.5°, 11.8°, 12.8°, 17.4°, 18.7°, 19.2°, 21.5°, 21.9°, and 24.5°.

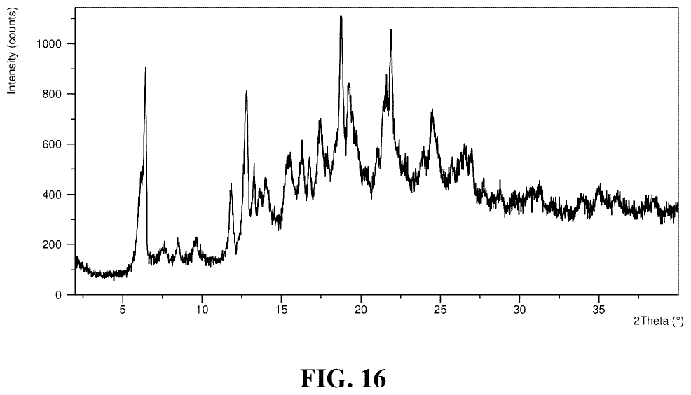

In some embodiments, the Compound 1, crystalline Form III is characterized by an XRPD pattern substantially as shown in FIG. 16.

In some embodiments, the Compound 1, crystalline Form III is characterized by a DSC thermogram having a melting onset at about 175° C.

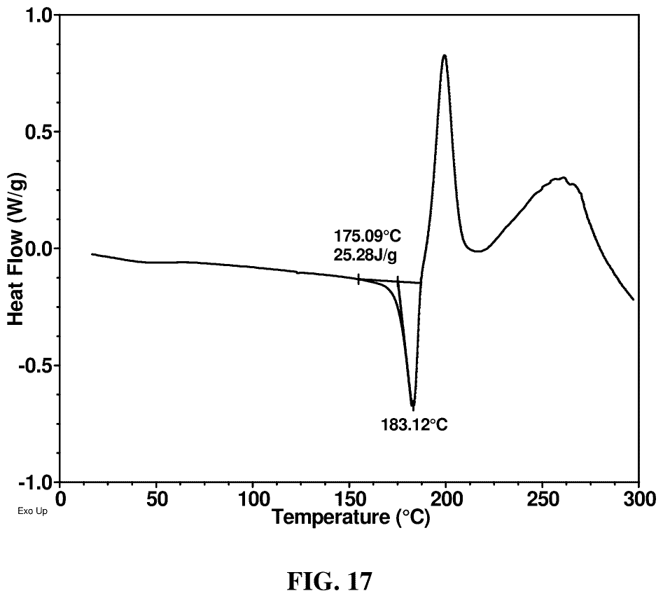

In some embodiments, the Compound 1, crystalline Form III is characterized by a DSC thermogram substantially as shown in FIG. 17.

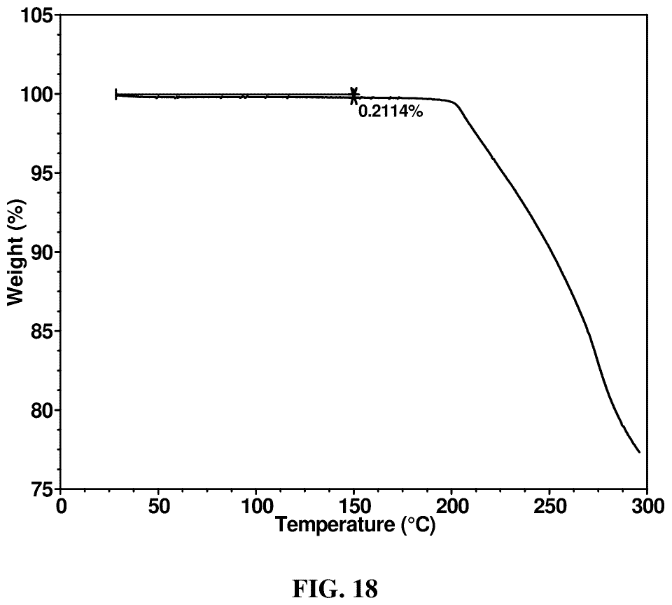

In some embodiments, the Compound 1, crystalline Form III is characterized by a TGA thermogram substantially as shown in FIG. 18.

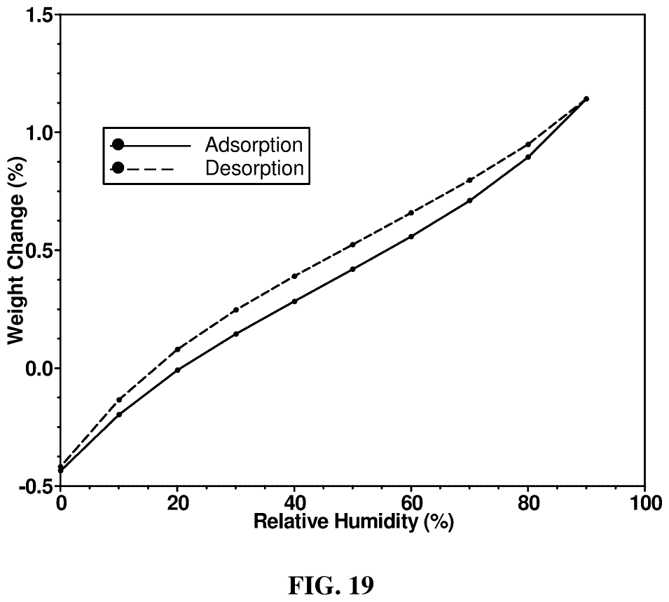

In some embodiments, the Compound 1, crystalline Form III is characterized by a DVS analysis substantially as shown in FIG. 19.

### Compound 1, Solvate Forms

In some embodiments, the present application provides a solvate form of Compound 1.

As used herein, the term “solvate” refers to a complex formed by the combining of a compound provided herein (e.g., Compound 1 or Compound 2), and a solvent or a crystalline solid containing amounts of a solvent incorporated within the crystal structure. As used herein, the term “solvate” includes hydrates. The term “hydrate” refers to the complex formed by the combining a compound provided herein (e.g., Compound 1 or Compound 2), and water.

In some embodiments, the Compound 1, solvate form is selected from an acetonitrile solvate form, a butyronitrile solvate form, a methyl ethyl ketone solvate form, a methyl isobutyl ketone solvate form, a toluene solvate form, a nitromethane solvate form, a diethyl ether solvate form, a cyclopentyl methyl ether solvate form, an anisole solvate form, a xylene solvate form, a dichloromethane solvate form, a chloroform solvate form, an acetophenone solvate form, a dichloroethane solvate form, a methyl acetate solvate form, an ethylacetate solvate form, an isopropyl acetate solvate form, a n-butyl acetate solvate form, and a cyclohexane solvate form.

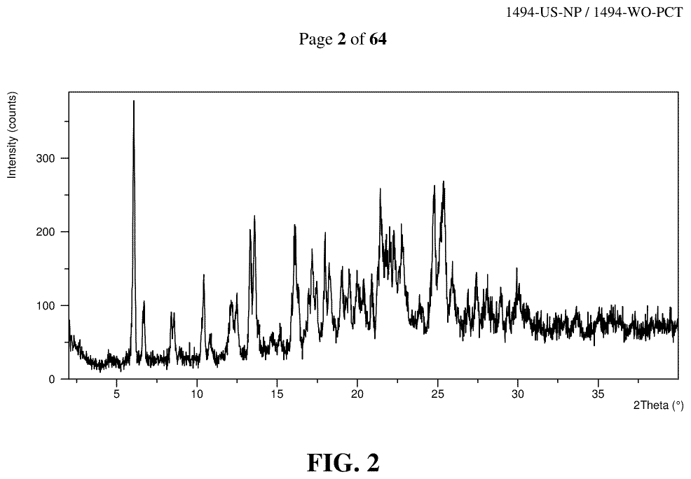

In some embodiments, the solvate form of Compound 1 is an acetonitrile solvate form. In some embodiments, the Compound 1, acetonitrile solvate form is characterized by an XRPD pattern substantially as shown in FIG. 2.

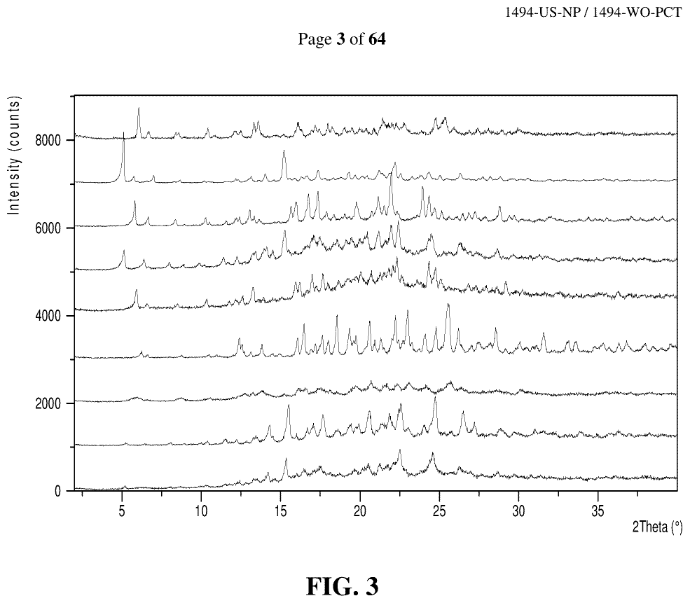

In some embodiments, the solvate form of Compound 1 is a butyronitrile solvate form. In some embodiments, the Compound 1, butyronitrile solvate form is characterized by an XRPD pattern substantially as shown in FIG. 3.

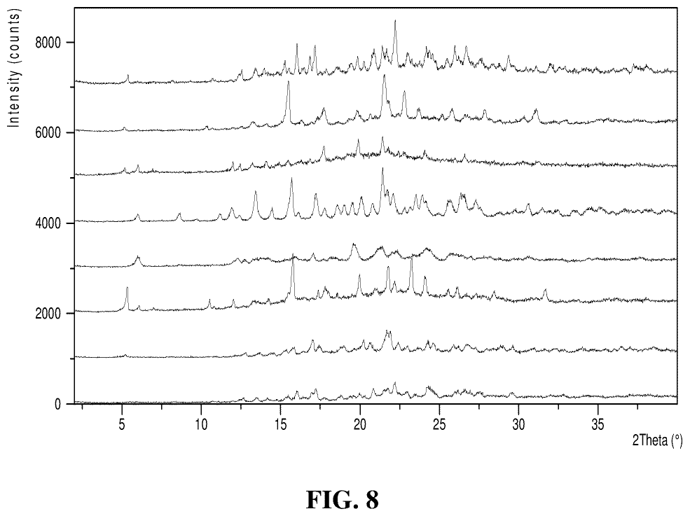

In some embodiments, the solvate form of Compound 1 is a methyl ethyl ketone solvate form. In some embodiments, the Compound 1, methyl ethyl ketone solvate form is characterized by an XRPD pattern substantially as shown in FIG. 8.

In some embodiments, the solvate form of Compound 1 is a methyl isobutyl ketone solvate form. In some embodiments, the solvate form of Compound 1 is methyl isobutyl ketone solvate form 1. In some embodiments, the Compound 1, methyl isobutyl ketone solvate form 1 is characterized by an XRPD pattern substantially as shown in FIG. 3. In some embodiments, the solvate form of Compound 1 is methyl isobutyl ketone solvate form 2. In some embodiments, the Compound 1, methyl isobutyl ketone solvate form 2 is characterized by an XRPD pattern substantially as shown in FIG. 3. In some embodiments, the solvate form of Compound 1 is methyl isobutyl ketone solvate form 3.

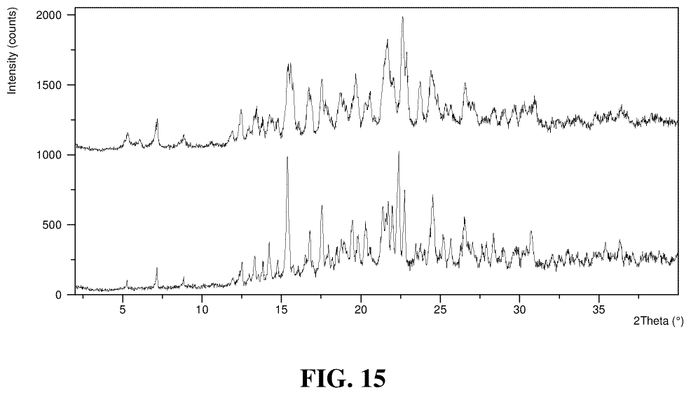

In some embodiments, the Compound 1, methyl isobutyl ketone solvate form 3 is characterized by an XRPD pattern substantially as shown in FIG. 15.

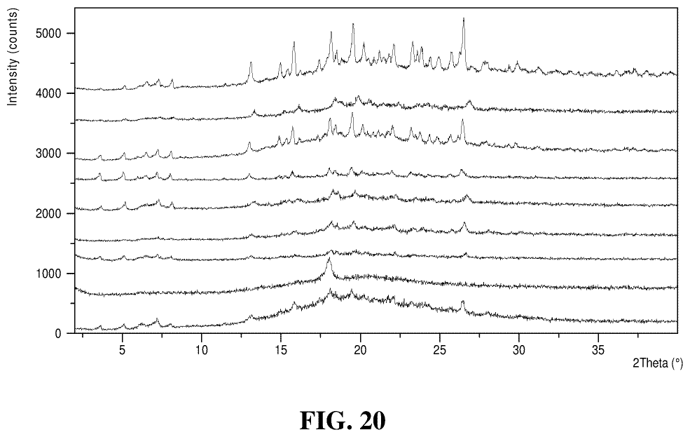

In some embodiments, the solvate form of Compound 1 is a toluene solvate form. In some embodiments, the solvate form of Compound 1 is toluene solvate form 1. In some embodiments, the Compound 1, toluene solvate form 1 is characterized by an XRPD pattern substantially as shown in FIG. 20. In some embodiments, the solvate form of Compound 1 is toluene solvate form 2. In some embodiments, the Compound 1, toluene solvate form 2 is characterized by an XRPD pattern substantially as shown in FIG. 3.

In some embodiments, the solvate form of Compound 1 is a nitromethane solvate form. In some embodiments, the Compound 1, nitromethane solvate form is characterized by an XRPD pattern substantially as shown in FIG. 3.

In some embodiments, the solvate form of Compound 1 is a diethyl ether solvate form. In some embodiments, the Compound 1, diethyl ether solvate form is characterized by an XRPD pattern substantially as shown in FIG. 3.

In some embodiments, the solvate form of Compound 1 is a cyclopentyl methyl ether solvate form. In some embodiments, the Compound 1, cyclopentyl methyl ether solvate form is characterized by an XRPD pattern substantially as shown in FIG. 8.

In some embodiments, the solvate form of Compound 1 is an anisole solvate form. In some embodiments, the solvate form of Compound 1 is anisole solvate form 1. In some embodiments, the Compound 1, anisole solvate form 1 is characterized by an XRPD pattern substantially as shown in FIG. 20. In some embodiments, the solvate form of Compound 1 is anisole solvate form 2. In some embodiments, the Compound 1, anisole solvate form 2 is characterized by an XRPD pattern substantially as shown in FIG. 3.

In some embodiments, the solvate form of Compound 1 is a xylene solvate form. In some embodiments, the Compound 1, xylene solvate is characterized by an XRPD pattern substantially as shown in FIG. 3.

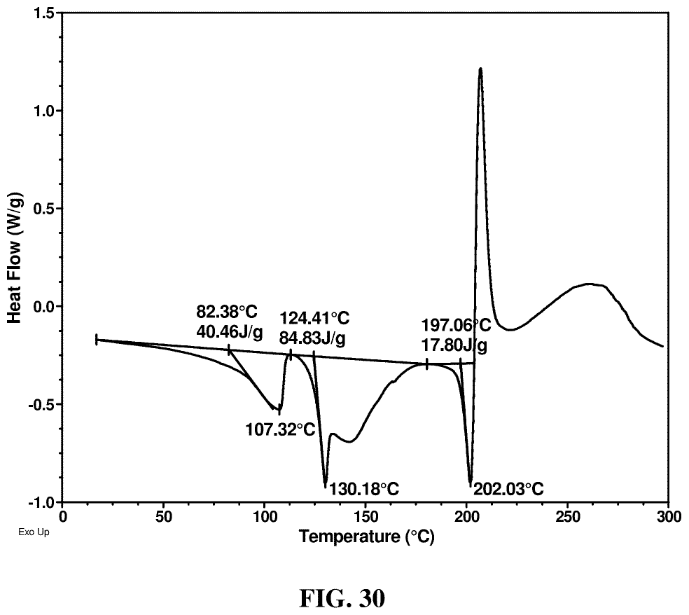

In some embodiments, the solvate form of Compound 1 is a dichloromethane solvate form. In some embodiments, the solvate form of Compound 1 is dichloromethane solvate form 1. In some embodiments, the Compound 1, dichloromethane solvate form 1 is characterized by an XRPD pattern substantially as shown in FIG. 30. In some embodiments, the solvate form of Compound 1 is dichloromethane solvate form 2. In some embodiments, the Compound 1, dichloromethane solvate form 2 is characterized by an XRPD pattern substantially as shown in FIG. 8.

In some embodiments, the solvate form of Compound 1 is a chloroform solvate form. In some embodiments, the solvate form of Compound 1 is chloroform solvate form 1. In some embodiments, the Compound 1, chloroform solvate form 1 is characterized by an XRPD pattern substantially as shown in FIG. 20. In some embodiments, the solvate form of Compound 1 is chloroform solvate form 2. In some embodiments, the Compound 1, chloroform solvate form 2 is characterized by an XRPD pattern substantially as shown in FIG. 8.

In some embodiments, the solvate form of Compound 1 is an acetophenone solvate form. In some embodiments, the Compound 1, acetophenone solvate form is characterized by an XRPD pattern substantially as shown in FIG. 8.

In some embodiments, the solvate form of Compound 1 is a dichloroethane solvate form. In some embodiments, the Compound 1, dichloroethane solvate form is characterized by an XRPD pattern substantially as shown in FIG. 8.

In some embodiments, the solvate form of Compound 1 is a methyl acetate solvate form. In some embodiments, the Compound 1, methyl acetate solvate form is characterized by an XRPD pattern substantially as shown in FIG. 20.

In some embodiments, the solvate form of Compound 1 is an ethyl acetate solvate form. In some embodiments, the solvate form of Compound 1 is ethyl acetate solvate form 1. In some embodiments, the Compound 1, ethyl acetate solvate form 1 is characterized by an XRPD pattern substantially as shown in FIG. 20. In some embodiments, the solvate form of Compound 1 is ethyl acetate solvate form 2. In some embodiments, the Compound 1, ethyl acetate solvate form 2 is characterized by an XRPD pattern substantially as shown in FIG. 8.

In some embodiments, the solvate form of Compound 1 is an isopropyl acetate solvate form. In some embodiments, the solvate form of Compound 1 is isopropyl acetate solvate form 1. In some embodiments, the Compound 1, isopropyl acetate solvate form 1 is characterized by an XRPD pattern substantially as shown in FIG. 20. In some embodiments, the solvate form of Compound 1 is isopropyl acetate solvate form 2. In some embodiments, the Compound 1, isopropyl acetate solvate form 2 is characterized by an XRPD pattern substantially as shown in FIG. 15.

In some embodiments, the solvate form of Compound 1 is a n-butyl acetate solvate form. In some embodiments, the solvate form of Compound 1 is n-butyl acetate solvate form 1. In some embodiments, the Compound 1, n-butyl acetate solvate form 1 is characterized by an XRPD pattern substantially as shown in FIG. 20. In some embodiments, the solvate form of Compound 1 is n-butyl acetate solvate form 2. In some embodiments, the Compound 1, n-butyl acetate solvate form 2 is characterized by an XRPD pattern substantially as shown in FIG. 8.

In some embodiments, the solvate form of Compound 1 is a cyclohexane solvate form. In some embodiments, the Compound 1, cyclohexane solvate form is characterized by an XRPD pattern substantially as shown in FIG. 20.

### Compound 1, Co-Crystal Forms

In some embodiments, the present application provides a co-crystal form of Compound 1.

As used herein, the term “co-crystal” refers to a crystalline material formed by combining a compound provided herein (e.g., Compound 1 or Compound 2) and one or more co-crystal formers (i.e., a molecule, ion, or atom). In certain instances, co-crystals may have improved properties as compared to the parent form (i.e., the free molecule, zwitterion, etc.) or a salt of the parent compound. Improved properties can include, but are not limited to, increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are known to those of skill in the art.

In some embodiments, the Compound 1 co-crystal provided herein is selected from the group consisting of a maleic acid co-crystal (i.e., “Compound 1, maleate co-crystal” or “Compound 1 maleate”), a succinic acid co-crystal (i.e., “Compound 1, succinate co-crystal” or “Compound 1 succinate”), an oxalic acid co-crystal (i.e., “Compound 1, oxalate co-crystal” or “Compound 1 oxalate”), a gentisic acid co-crystal (i.e., “Compound 1, gentisate co-crystal” or “Compound 1 gentisate”), an L-tartaric acid co-crystal (i.e., “Compound 1, L-tartrate co-crystal” or “Compound 1 L-tartrate”), and a vanillic acid co-crystal (i.e., “Compound 1, vanillate co-crystal” or “Compound 1 vanillate”).

In some embodiments, the co-crystal provided herein is a Compound 1, maleic acid co-crystal. In some embodiments, the Compound 1, maleic acid co-crystal can be prepared with 1.0 to 2.0 equivalents of maleic acid.

In some embodiments, the Compound 1, maleic acid co-crystal has at least one XRPD peak, in terms of 2-theta±0.2°, selected from 6.0°, 6.7°, 12.3°, 13.4°, 16.1°, 20.1°, 21.8°, 22.4°, and 24.8°.

In some embodiments, the Compound 1, maleic acid co-crystal has at least two XRPD peaks, in terms of 2-theta±0.2°, selected from 6.0°, 6.7°, 12.3°, 13.4°, 16.1°, 20.1°, 21.8°, 22.4°, and 24.8°.

In some embodiments, the Compound 1, maleic acid co-crystal has at least three XRPD peaks, in terms of 2-theta±0.2°, selected from 6.0°, 6.7°, 12.3°, 13.4°, 16.1°, 20.1°, 21.8°, 22.4°, and 24.8°.

In some embodiments, the Compound 1, maleic acid co-crystal has at least four XRPD peaks, in terms of 2-theta±0.2°, selected from 6.0°, 6.7°, 12.3°, 13.4°, 16.1°, 20.1°, 21.8°, 22.4°, and 24.8°.

In some embodiments, the Compound 1, maleic acid co-crystal has at least five XRPD peaks, in terms of 2-theta±0.2°, selected from 6.0°, 6.7°, 12.3°, 13.4°, 16.1°, 20.1°, 21.8°, 22.4°, and 24.8°.

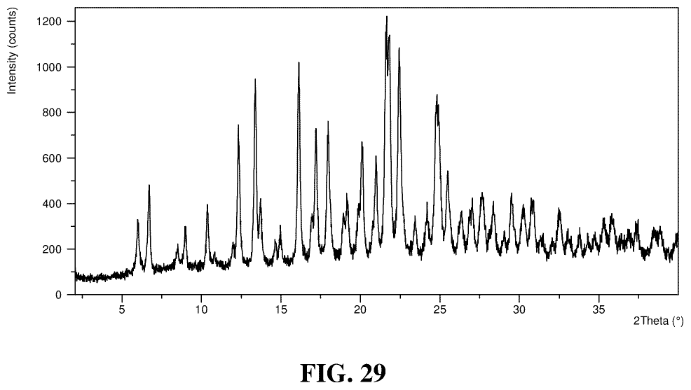

In some embodiments, the Compound 1, maleic acid co-crystal is characterized by an XRPD pattern substantially as shown in FIG. 29.

In some embodiments, the Compound 1, maleic acid co-crystal is characterized by a DSC thermogram having an endothermic transition at about 82° C.

In some embodiments, the Compound 1, maleic acid co-crystal is characterized by a DSC thermogram having an endothermic transition at about 124° C.

In some embodiments, the Compound 1, maleic acid co-crystal is characterized by a DSC thermogram having an endothermic transition at about 197° C.

In some embodiments, the Compound 1, maleic acid co-crystal is characterized by a DSC thermogram having at least one endothermic transition selected from the group consisting of about 82° C., about 124° C., and about 197° C.

In some embodiments, the Compound 1, maleic acid co-crystal is characterized by a DSC thermogram having at least two endothermic transitions selected from the group consisting of about 82° C., about 124° C., and about 197° C.

In some embodiments, the Compound 1, maleic acid co-crystal is characterized by a DSC thermogram having at least three endothermic transitions selected from the group consisting of about 82° C., about 124° C., and about 197° C.

In some embodiments, the Compound 1, maleic acid co-crystal is characterized by a DSC thermogram having an endothermic transition at about 82° C., 124° C., and 197° C.

In some embodiments, the Compound 1, maleic acid co-crystal is characterized by a DSC thermogram substantially as shown in FIG. 30.

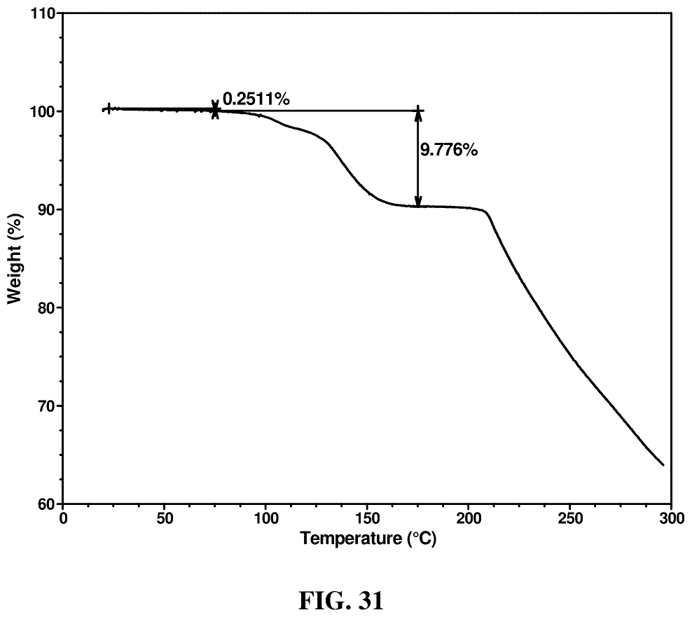

In some embodiments, the Compound 1, maleic acid co-crystal is characterized by a TGA thermogram substantially as shown in FIG. 31.

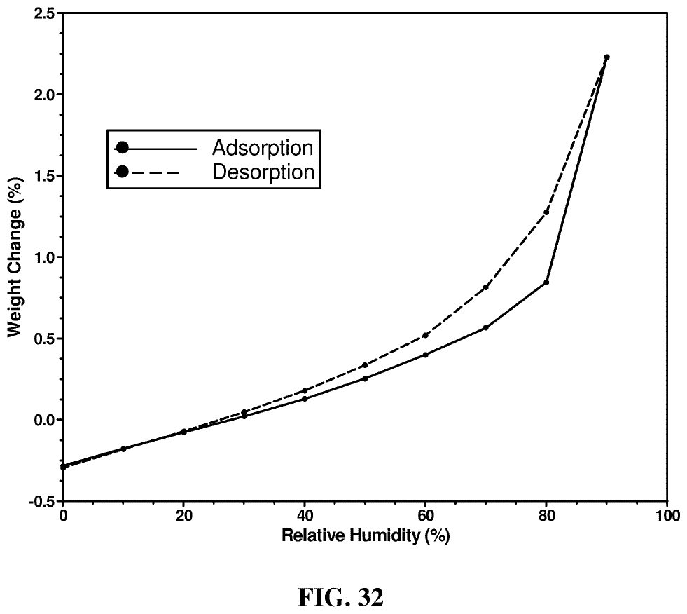

In some embodiments, the Compound 1, maleic acid co-crystal is characterized by a DVS analysis substantially as shown in FIG. 32.

In some embodiments, the co-crystal provided herein is a Compound 1, succinic acid co-crystal. In some embodiments, the Compound 1, succinic acid co-crystal can be prepared with 1.0 to 2.0 equivalents of succinic acid.

In some embodiments, the Compound 1, succinic acid co-crystal has at least one XRPD peak, in terms of 2-theta±0.2°, selected from 13.5°, 16.3°, 17.4°, 18.1°, 20.2°, 22.0°, 22.7°, 24.9°, and 25.7°.

In some embodiments, the Compound 1, succinic acid co-crystal has at least two XRPD peaks, in terms of 2-theta±0.2°, selected from 13.5°, 16.3°, 17.4°, 18.1°, 20.2°, 22.0°, 22.7°, 24.9°, and 25.7°.

In some embodiments, the Compound 1, succinic acid co-crystal has at least three XRPD peaks, in terms of 2-theta±0.2°, selected from 13.5°, 16.3°, 17.4°, 18.1°, 20.2°, 22.0°, 22.7°, 24.9°, and 25.7°.

In some embodiments, the Compound 1, succinic acid co-crystal has at least four XRPD peaks, in terms of 2-theta±0.2°, selected from 13.5°, 16.3°, 17.4°, 18.1°, 20.2°, 22.0°, 22.7°, 24.9°, and 25.7°.

In some embodiments, the Compound 1, succinic acid co-crystal has at least five XRPD peaks, in terms of 2-theta±0.2°, selected from 13.5°, 16.3°, 17.4°, 18.1°, 20.2°, 22.0°, 22.7°, 24.9°, and 25.7°.

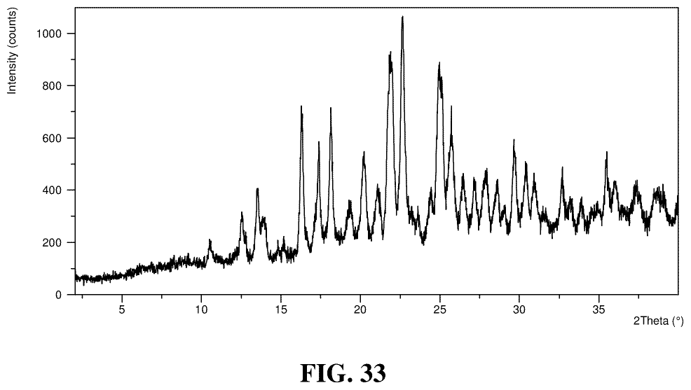

In some embodiments, the Compound 1, succinic acid co-crystal is characterized by an XRPD pattern substantially as shown in FIG. 33.

In some embodiments, the Compound 1, succinic acid co-crystal is characterized by a DSC thermogram having an endothermic transition at about 39° C.

In some embodiments, the Compound 1, succinic acid co-crystal is characterized by a DSC thermogram having an endothermic transition at about 163° C.

In some embodiments, the Compound 1, succinic acid co-crystal is characterized by a DSC thermogram having an endothermic transition at about 164° C.

In some embodiments, the Compound 1, succinic acid co-crystal is characterized by a DSC thermogram having at least one endothermic transition selected from the group consisting of about 39° C., about 163° C., and about 164° C.

In some embodiments, the Compound 1, succinic acid co-crystal is characterized by a DSC thermogram having at least two endothermic transitions selected from the group consisting of about 39° C., about 163° C., and about 164° C.

In some embodiments, the Compound 1, succinic acid co-crystal is characterized by a DSC thermogram having at least three endothermic transitions selected from the group consisting of about 39° C., about 163° C., and about 164° C.

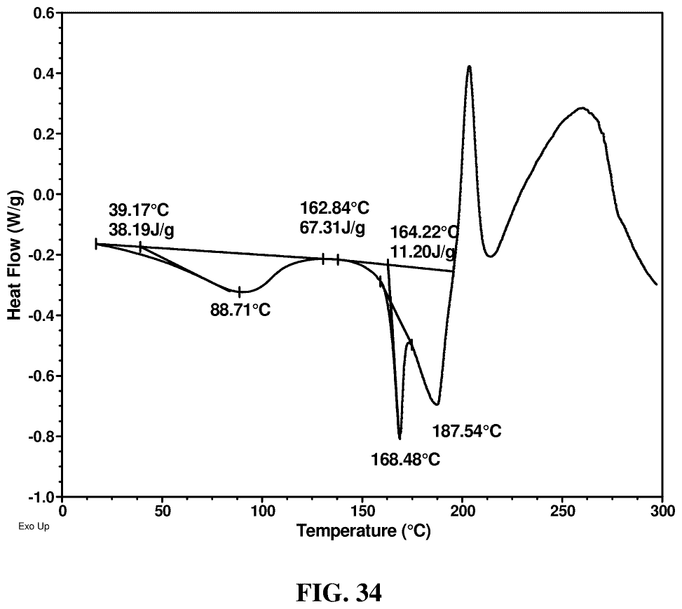

In some embodiments, the Compound 1, succinic acid co-crystal is characterized by a DSC thermogram substantially as shown in FIG. 34.

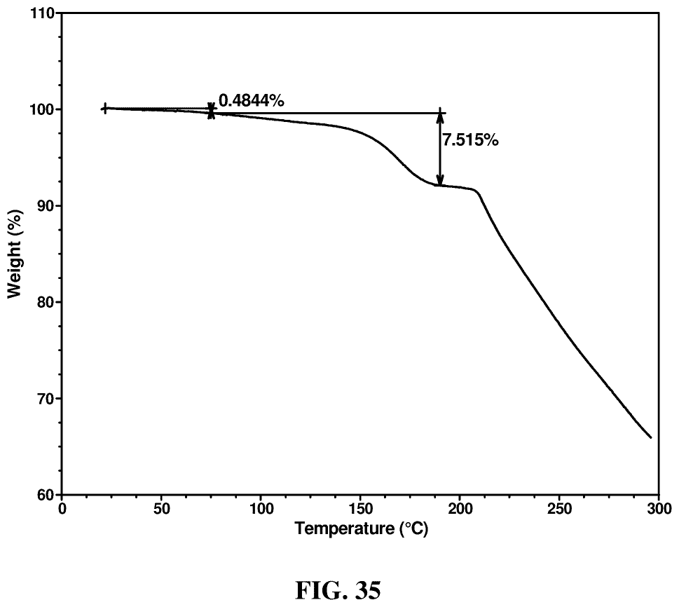

In some embodiments, the Compound 1, succinic acid co-crystal is characterized by a TGA thermogram substantially as shown in FIG. 35.

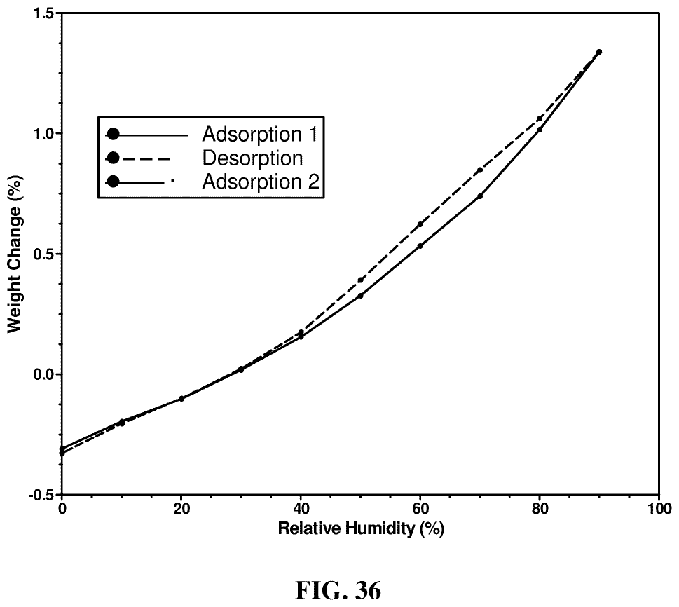

In some embodiments, the Compound 1, succinic acid co-crystal is characterized by a DVS analysis substantially as shown in FIG. 36.

In some embodiments, the co-crystal provided herein is a Compound 1, oxalic acid co-crystal.

In some embodiments, the Compound 1, oxalic acid co-crystal has at least one XRPD peak, in terms of 2-theta±0.2°, selected from 14.4°, 17.1°, 17.4°, 19.7°, 20.3°, 21.9°, 22.9°, 24.4°, and 26.7°.

In some embodiments, the Compound 1, oxalic acid co-crystal has at least two XRPD peaks, in terms of 2-theta±0.2°, selected from 14.4°, 17.1°, 17.4°, 19.7°, 20.3°, 21.9°, 22.9°, 24.4°, and 26.7°.

In some embodiments, the Compound 1, oxalic acid co-crystal has at least three XRPD peaks, in terms of 2-theta±0.2°, selected from 14.4°, 17.1°, 17.4°, 19.7°, 20.3°, 21.9°, 22.9°, 24.4°, and 26.7°.

In some embodiments, the Compound 1, oxalic acid co-crystal has at least four XRPD peaks, in terms of 2-theta±0.2°, selected from 14.4°, 17.1°, 17.4°, 19.7°, 20.3°, 21.9°, 22.9°, 24.4°, and 26.7°.

In some embodiments, the Compound 1, oxalic acid co-crystal has at least five XRPD peaks, in terms of 2-theta±0.2°, selected from 14.4°, 17.1°, 17.4°, 19.7°, 20.3°, 21.9°, 22.9°, 24.4°, and 26.7°.

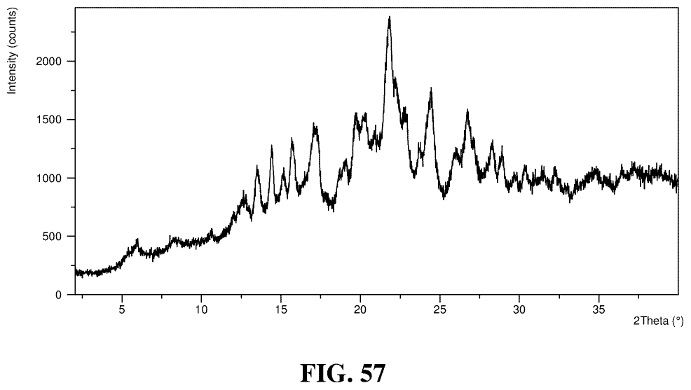

In some embodiments, the Compound 1, oxalic acid co-crystal is characterized by an XRPD pattern substantially as shown in FIG. 57.

In some embodiments, the Compound 1, oxalic acid co-crystal is characterized by a DSC thermogram having an endothermic transition at about 82° C.

In some embodiments, the Compound 1, oxalic acid co-crystal is characterized by a DSC thermogram having an endothermic transition at about 165° C.

In some embodiments, the Compound 1, oxalic acid co-crystal is characterized by a DSC thermogram having an endothermic transition at about 187° C.

In some embodiments, the Compound 1, oxalic acid co-crystal is characterized by a DSC thermogram having at least one endothermic transition selected from the group consisting of about 82° C., about 165° C., and about 187° C.

In some embodiments, the Compound 1, oxalic acid co-crystal is characterized by a DSC thermogram having at least two endothermic transitions selected from the group consisting of about 82° C., about 165° C., and about 187° C.

In some embodiments, the Compound 1, oxalic acid co-crystal is characterized by a DSC thermogram having at least three endothermic transitions selected from the group consisting of about 82° C., about 165° C., and about 187° C.

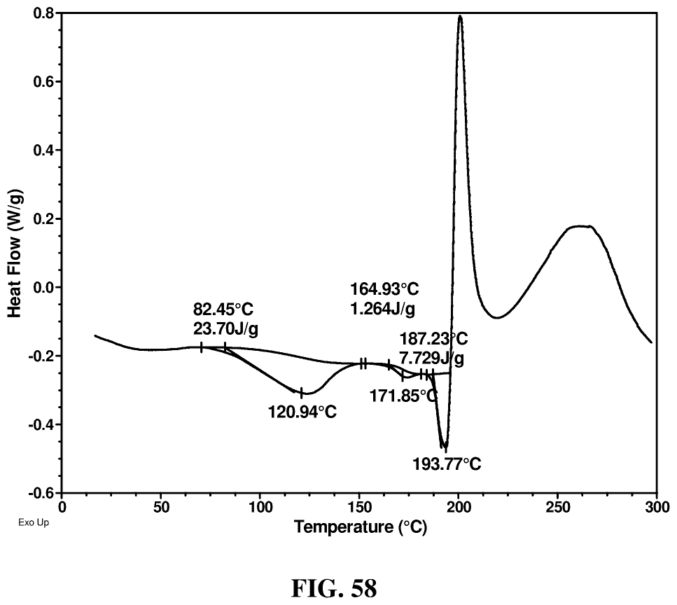

In some embodiments, the Compound 1, oxalic acid co-crystal is characterized by a DSC thermogram substantially as shown in FIG. 58.

In some embodiments, the co-crystal provided herein is a Compound 1, gentisic acid co-crystal.

In some embodiments, the Compound 1, gentisic acid co-crystal has at least one XRPD peak, in terms of 2-theta±0.2°, selected from 5.3°, 5.9°, 12.7°, 14.3°, 15.8°, 16.9°, 20.2°, 21.7°, and 26.7°.

In some embodiments, the Compound 1, gentisic acid co-crystal has at least two XRPD peaks, in terms of 2-theta±0.2°, selected from 5.3°, 5.9°, 12.7°, 14.3°, 15.8°, 16.9°, 20.2°, 21.7°, and 26.7°.

In some embodiments, the Compound 1, gentisic acid co-crystal has at least three XRPD peaks, in terms of 2-theta±0.2°, selected from 5.3°, 5.9°, 12.7°, 14.3°, 15.8°, 16.9°, 20.2°, 21.7°, and 26.7°.

In some embodiments, the Compound 1, gentisic acid co-crystal has at least four XRPD peaks, in terms of 2-theta±0.2°, selected from 5.3°, 5.9°, 12.7°, 14.3°, 15.8°, 16.9°, 20.2°, 21.7°, and 26.7°.

In some embodiments, the Compound 1, gentisic acid co-crystal has at least five XRPD peaks, in terms of 2-theta±0.2°, selected from 5.3°, 5.9°, 12.7°, 14.3°, 15.8°, 16.9°, 20.2°, 21.7°, and 26.7°.

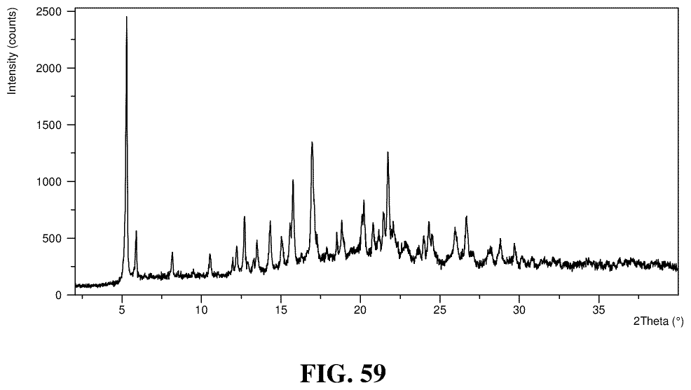

In some embodiments, the Compound 1, gentisic acid co-crystal is characterized by an XRPD pattern substantially as shown in FIG. 59.

In some embodiments, the Compound 1, gentisic acid co-crystal is characterized by a DSC thermogram having an endothermic transition at about 77° C.

In some embodiments, the Compound 1, gentisic acid co-crystal is characterized by a DSC thermogram having an endothermic transition at about 120° C.

In some embodiments, the Compound 1, gentisic acid co-crystal is characterized by a DSC thermogram having at least one endothermic transition selected from the group consisting of about 77° C. and about 120° C.

In some embodiments, the Compound 1, gentisic acid co-crystal is characterized by a DSC thermogram having at least two endothermic transitions selected from the group consisting of about 77° C. and about 120° C.

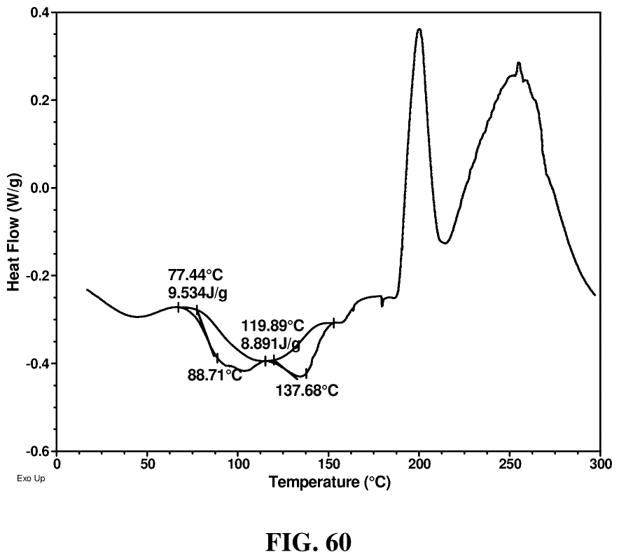

In some embodiments, the Compound 1, gentisic acid co-crystal is characterized by a DSC thermogram substantially as shown in FIG. 60.

In some embodiments, the co-crystal provided herein is a Compound 1, L-tartaric acid co-crystal.

In some embodiments, the Compound 1, L-tartaric acid co-crystal has at least one XRPD peak, in terms of 2-theta±0.2°, selected from 5.3°, 15.7°, 17.0°, 18.9°, 20.2°, 20.8°, 21.8°, 26.7°, and 29.8°.

In some embodiments, the Compound 1, L-tartaric acid co-crystal has at least two XRPD peaks, in terms of 2-theta±0.2°, selected from 5.3°, 15.7°, 17.0°, 18.9°, 20.2°, 20.8°, 21.8°, 26.7°, and 29.8°.

In some embodiments, the Compound 1, L-tartaric acid co-crystal has at least three XRPD peaks, in terms of 2-theta±0.2°, selected from 5.3°, 15.7°, 17.0°, 18.9°, 20.2°, 20.8°, 21.8°, 26.7°, and 29.8°.

In some embodiments, the Compound 1, L-tartaric acid co-crystal has at least four XRPD peaks, in terms of 2-theta±0.2°, selected from 5.3°, 15.7°, 17.0°, 18.9°, 20.2°, 20.8°, 21.8°, 26.7°, and 29.8°.

In some embodiments, the Compound 1, L-tartaric acid co-crystal has at least five XRPD peaks, in terms of 2-theta±0.2°, selected from 5.3°, 15.7°, 17.0°, 18.9°, 20.2°, 20.8°, 21.8°, 26.7°, and 29.8°.

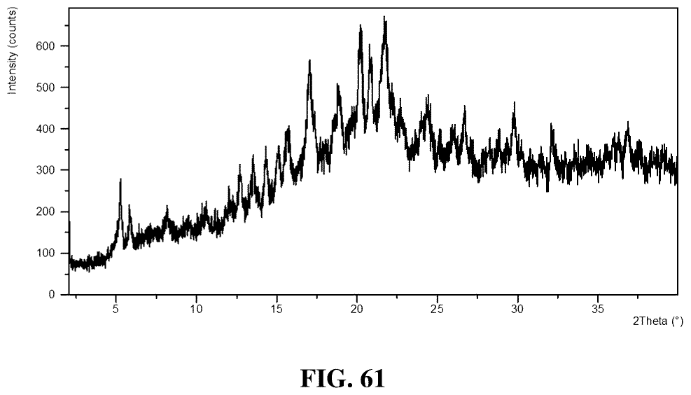

In some embodiments, the Compound 1, L-tartaric acid co-crystal is characterized by an XRPD pattern substantially as shown in FIG. 61.

In some embodiments, the Compound 1, L-tartaric acid co-crystal is characterized by a DSC thermogram having an endothermic transition at about 109° C.

In some embodiments, the Compound 1, L-tartaric acid co-crystal is characterized by a DSC thermogram having an endothermic transition at about 162° C.

In some embodiments, the Compound 1, L-tartaric acid co-crystal is characterized by a DSC thermogram having at least one endothermic transition selected from the group consisting of about 109° C. and about 162° C.

In some embodiments, the Compound 1, L-tartaric acid co-crystal is characterized by a DSC thermogram having at least two endothermic transitions selected from the group consisting of about 109° C. and about 162° C.

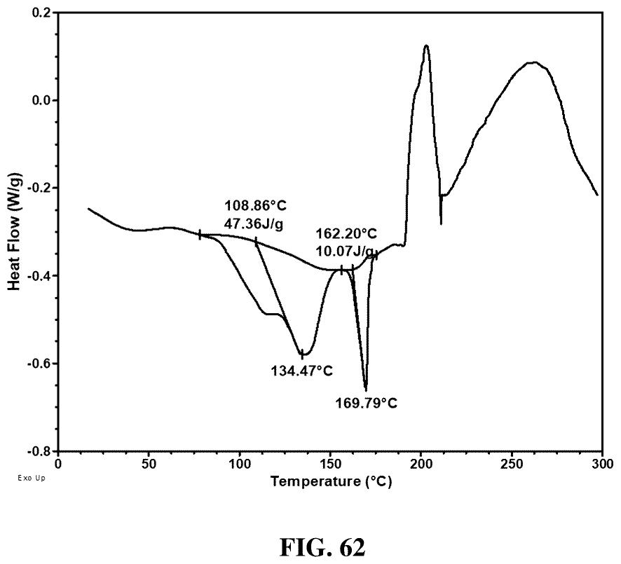

In some embodiments, the Compound 1, L-tartaric acid co-crystal is characterized by a DSC thermogram substantially as shown in FIG. 62.

In some embodiments, the co-crystal provided herein is a Compound 1, vanillic acid co-crystal.

In some embodiments, the Compound 1, vanillic acid co-crystal has at least one XRPD peak, in terms of 2-theta±0.2°, selected from 5.2°, 12.7°, 13.5°, 14.4°, 15.7°, 17.0°, 20.1°, 21.7°, and 26.7°.

In some embodiments, the Compound 1, vanillic acid co-crystal has at least two XRPD peaks, in terms of 2-theta±0.2°, selected from 5.2°, 12.7°, 13.5°, 14.4°, 15.7°, 17.0°, 20.1°, 21.7°, and 26.7°.

In some embodiments, the Compound 1, vanillic acid co-crystal has at least three XRPD peaks, in terms of 2-theta±0.2°, selected from 5.2°, 12.7°, 13.5°, 14.4°, 15.7°, 17.0°, 20.1°, 21.7°, and 26.7°.

In some embodiments, the Compound 1, vanillic acid co-crystal has at least four XRPD peaks, in terms of 2-theta±0.2°, selected from 5.2°, 12.7°, 13.5°, 14.4°, 15.7°, 17.0°, 20.1°, 21.7°, and 26.7°.

In some embodiments, the Compound 1, vanillic acid co-crystal has at least five XRPD peaks, in terms of 2-theta±0.2°, selected from 5.2°, 12.7°, 13.5°, 14.4°, 15.7°, 17.0°, 20.1°, 21.7°, and 26.7°.

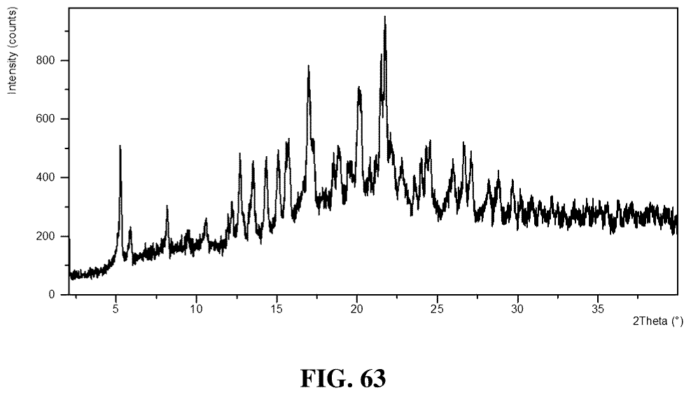

In some embodiments, the Compound 1, vanillic acid co-crystal is characterized by an XRPD pattern substantially as shown in FIG. 63.

In some embodiments, the Compound 1, vanillic acid co-crystal is characterized by a DSC thermogram having an endothermic transition at about 102° C.

In some embodiments, the Compound 1, vanillic acid co-crystal is characterized by a DSC thermogram having an endothermic transition at about 132° C.

In some embodiments, the Compound 1, vanillic acid co-crystal is characterized by a DSC thermogram having an endothermic transition at about 184° C.

In some embodiments, the Compound 1, vanillic acid co-crystal is characterized by a DSC thermogram having at least one endothermic transition selected from the group consisting of about 102° C., about 132° C., and about 184° C.

In some embodiments, the Compound 1, vanillic acid co-crystal is characterized by a DSC thermogram having at least two endothermic transitions selected from the group consisting of about 102° C., about 132° C., and about 184° C.

In some embodiments, the Compound 1, vanillic acid co-crystal is characterized by a DSC thermogram having at least three endothermic transitions selected from the group consisting of about 102° C., about 132° C., and about 184° C.

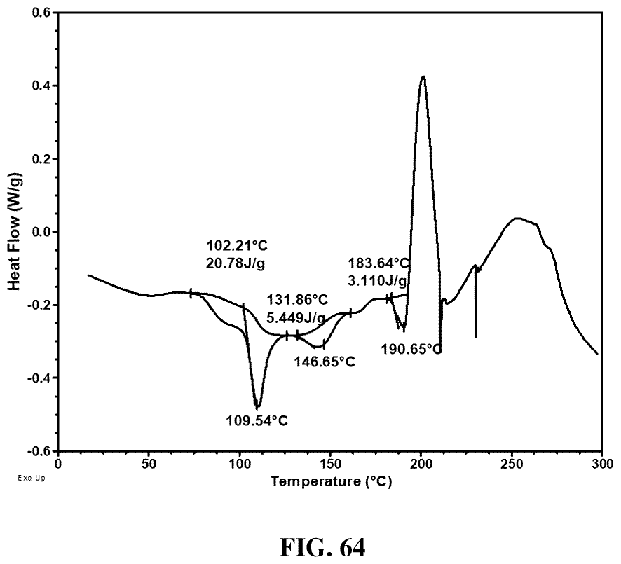

In some embodiments, the Compound 1, vanillic acid co-crystal is characterized by a DSC thermogram substantially as shown in FIG. 64.

### Compound 1, Material A

In some embodiments, the present application provides Compound 1, Material A.

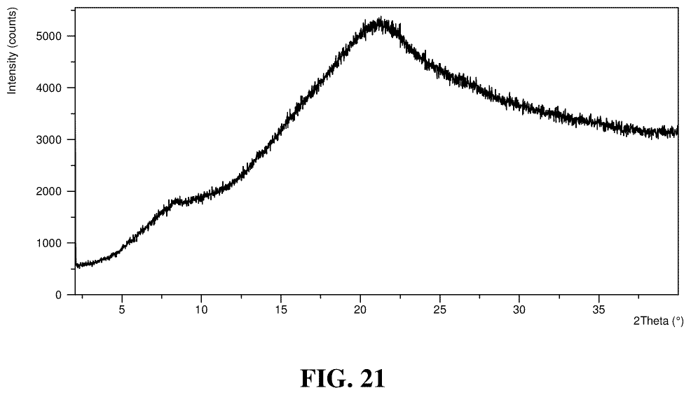

In some embodiments, the Compound 1, Material A is characterized by an XRPD pattern substantially as shown in FIG. 21.

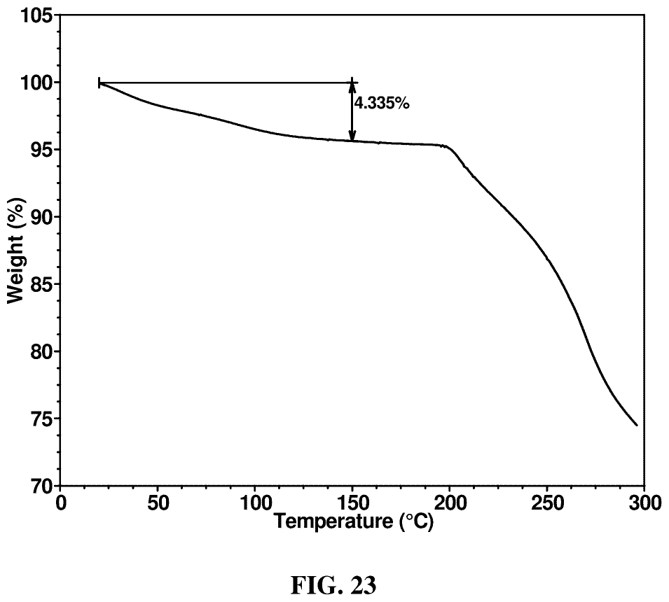

In some embodiments, the Compound 1, Material A is characterized by a TGA thermogram substantially as shown in FIG. 23.

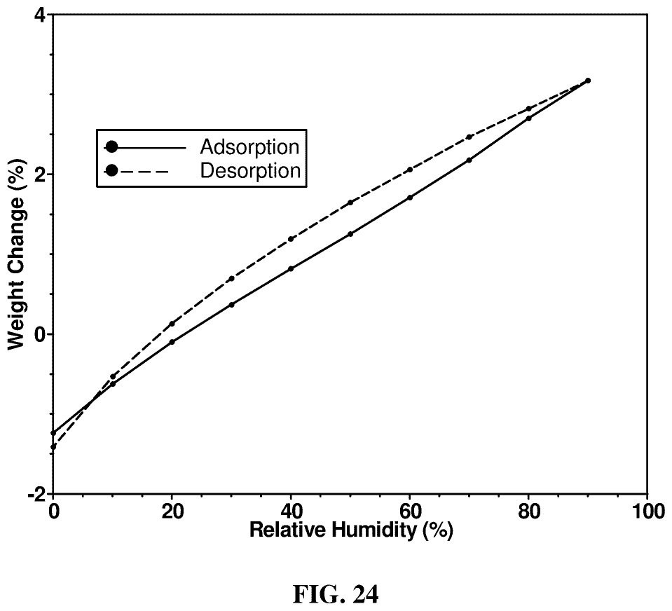

In some embodiments, the Compound 1, Material A is characterized by a DVS analysis substantially as shown in FIG. 24.

### Compound 1, Amorphous Form

In some embodiments, the present application provides an amorphous form of Compound 1.

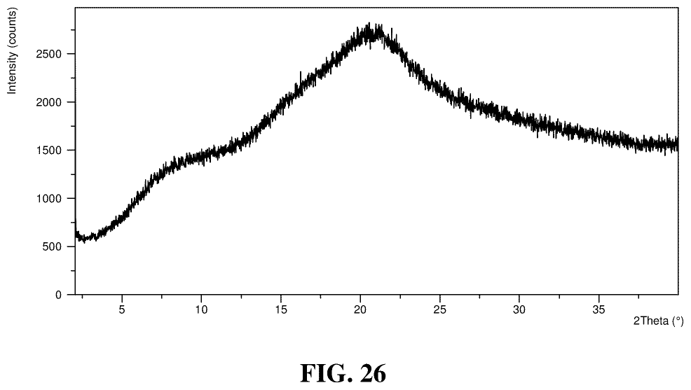

In some embodiments, the Compound 1, amorphous form is characterized by an XRPD pattern substantially as shown in FIG. 26.

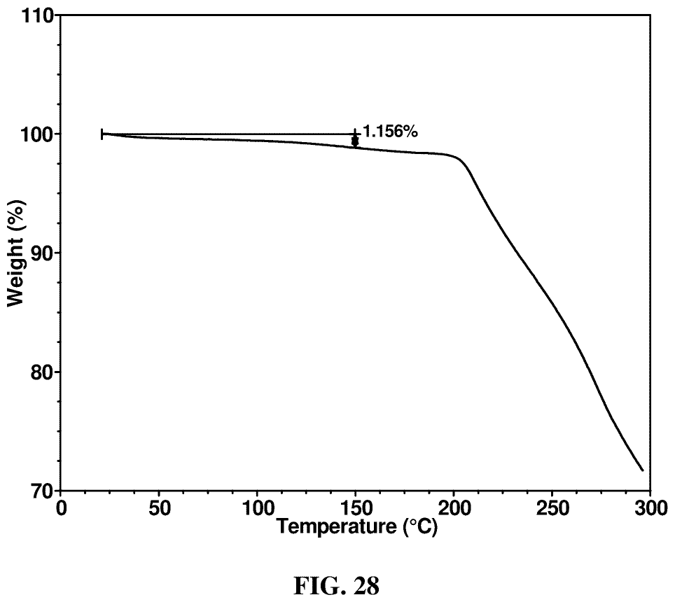

In some embodiments, the Compound 1, amorphous form is characterized by a TGA thermogram substantially as shown in FIG. 28.

### Compound 2, Crystalline Forms

In some embodiments, the present application provides a crystalline form of tert-butyl 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-3-((di-tert-butoxyphosphoryl)oxy)-5-methylphenyl)acetate (i.e., Compound 2).

In some embodiments, the crystalline form of Compound 2 provided herein is selected from crystalline Form I, crystalline Form II, and crystalline Form III.

In some embodiments, the crystalline form of Compound 2 provided herein is Compound 2, crystalline Form I.

In some embodiments, the Compound 2, crystalline Form I has at least one XRPD peak, in terms of 2-theta±0.2°, selected from 11.9°, 15.3°, 17.2°, 17.9°, 18.2°, 18.9°, 19.5°, 20.0°, and 21.1°.

In some embodiments, the Compound 2, crystalline Form I has at least two XRPD peaks, in terms of 2-theta±0.2°, selected from 11.9°, 15.3°, 17.2°, 17.9°, 18.2°, 18.9°, 19.5°, 20.0°, and 21.1°.

In some embodiments, the Compound 2, crystalline Form I has at least three XRPD peaks, in terms of 2-theta±0.2°, selected from 11.9°, 15.3°, 17.2°, 17.9°, 18.2°, 18.9°, 19.5°, 20.0°, and 21.1°.

In some embodiments, the Compound 2, crystalline Form I has at least four XRPD peaks, in terms of 2-theta±0.2°, selected from 11.9°, 15.3°, 17.2°, 17.9°, 18.2°, 18.9°, 19.5°, 20.0°, and 21.1°.

In some embodiments, the Compound 2, crystalline Form I has at least five XRPD peaks, in terms of 2-theta±0.2°, selected from 11.9°, 15.3°, 17.2°, 17.9°, 18.2°, 18.9°, 19.5°, 20.0°, and 21.1°.

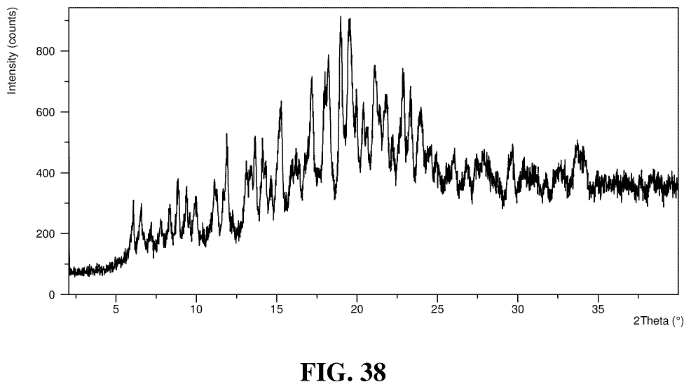

In some embodiments, the Compound 2, crystalline Form I is characterized by an XRPD pattern substantially as shown in FIG. 38.

In some embodiments, the Compound 2, crystalline Form I is characterized by a DSC thermogram having an endothermic event at about 79° C. In some embodiments, the Compound 2, crystalline Form I is characterized by a DSC thermogram having an endothermic transition at about 115° C. In some embodiments, the Compound 2, crystalline Form I is characterized by a DSC thermogram having an endothermic transition at about 121° C. In some embodiments, the Compound 2, crystalline Form I is characterized by a DSC thermogram having an endothermic transition at about 142° C.

In some embodiments, the Compound 2, crystalline Form I is characterized by a DSC thermogram having at least one endothermic transition selected from about 79° C., about 115° C., about 121° C., and about 142° C. In some embodiments, the Compound 2, crystalline Form I is characterized by a DSC thermogram having at least two endothermic transitions selected from about 79° C., about 115° C., about 121° C., and about 142° C. In some embodiments, the Compound 2, crystalline Form I is characterized by a DSC thermogram having at least three endothermic transitions selected from about 79° C., about 115° C., about 121° C., and about 142° C. In some embodiments, the Compound 2, crystalline Form I is characterized by a DSC thermogram having at least four endothermic transitions selected from about 79° C., about 115° C., about 121° C., and about 142° C.

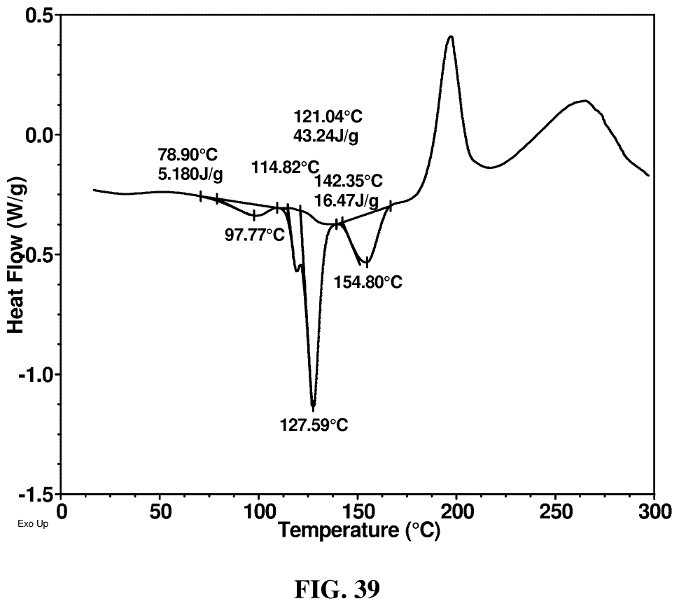

In some embodiments, the Compound 2, crystalline Form I is characterized by a DSC thermogram substantially as shown in FIG. 39.

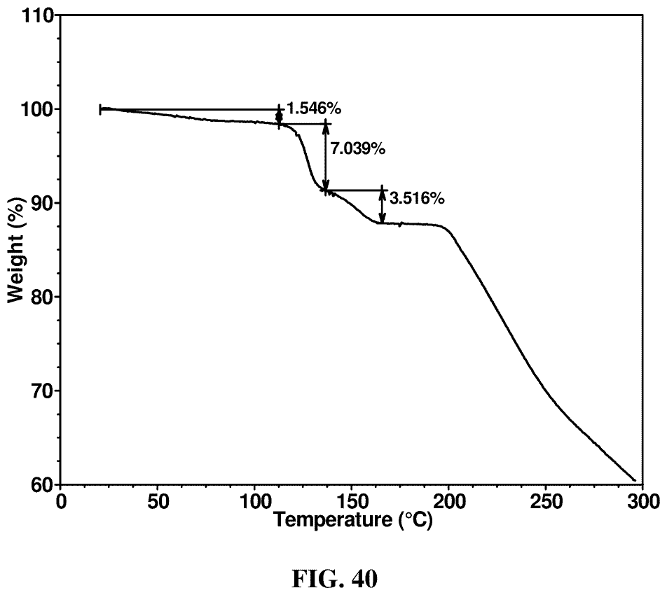

In some embodiments, the Compound 2, crystalline Form I is characterized by a TGA thermogram substantially as shown in FIG. 40.

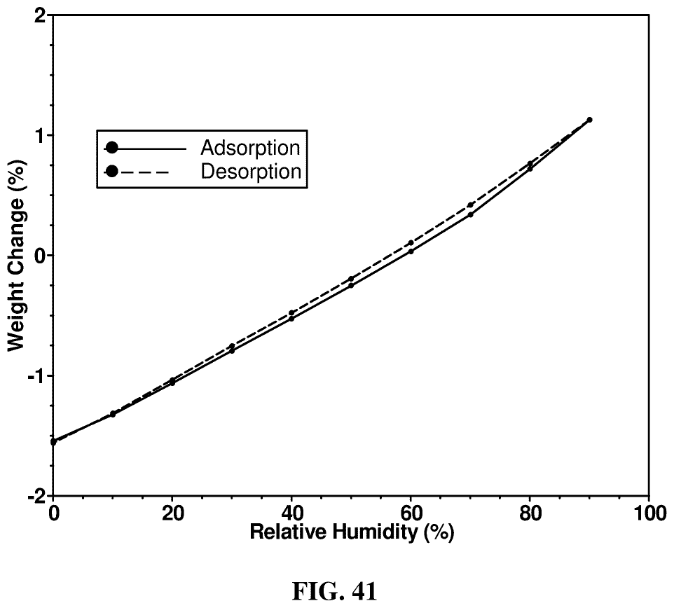

In some embodiments, the Compound 2, crystalline Form I is characterized by a DVS analysis substantially as shown in FIG. 41.

In some embodiments, the crystalline form of Compound 2 provided herein is Compound 2, crystalline Form II.

In some embodiments, the Compound 2, crystalline Form II has at least one XRPD peak, in terms of 2-theta±0.2°, selected from 15.0°, 16.9°, 17.7°, 19.3°, 20.2°, 20.8°, 21.3°, 23.1°, and 23.6°.

In some embodiments, the Compound 2, crystalline Form II has at least two XRPD peaks, in terms of 2-theta±0.2°, selected from 15.0°, 16.9°, 17.7°, 19.3°, 20.2°, 20.8°, 21.3°, 23.1°, and 23.6°.

In some embodiments, the Compound 2, crystalline Form II has at least three XRPD peaks, in terms of 2-theta±0.2°, selected from 15.0°, 16.9°, 17.7°, 19.3°, 20.2°, 20.8°, 21.3°, 23.1°, and 23.6°.

In some embodiments, the Compound 2, crystalline Form II has at least four XRPD peaks, in terms of 2-theta±0.2°, selected from 15.0°, 16.9°, 17.7°, 19.3°, 20.2°, 20.8°, 21.3°, 23.1°, and 23.6°.

In some embodiments, the Compound 2, crystalline Form II has at least five XRPD peaks, in terms of 2-theta±0.2°, selected from 15.0°, 16.9°, 17.7°, 19.3°, 20.2°, 20.8°, 21.3°, 23.1°, and 23.6°.

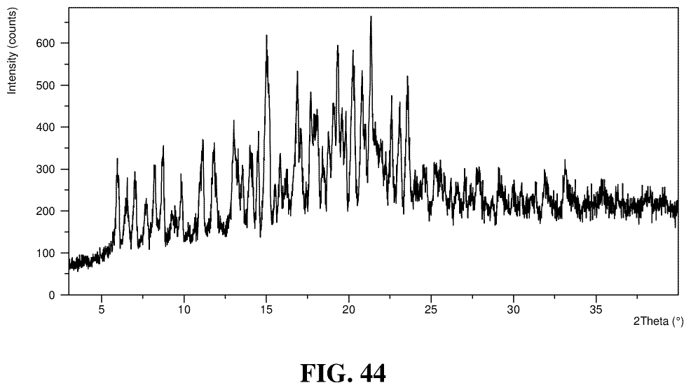

In some embodiments, the Compound 2, crystalline Form II is characterized by an XRPD pattern substantially as shown in FIG. 44.

In some embodiments, the Compound 2, crystalline Form II is characterized by a DSC thermogram having an endothermic event at about 119° C. In some embodiments, the Compound 2, crystalline Form II is characterized by a DSC thermogram having an endothermic transition at about 130° C.

In some embodiments, the Compound 2, crystalline Form II is characterized by a DSC thermogram having at least one endothermic transition selected from about 119° C. and about 130° C. In some embodiments, the Compound 2, crystalline Form II is characterized by a DSC thermogram having at least two endothermic transitions selected from about 119° C. and about 130° C.

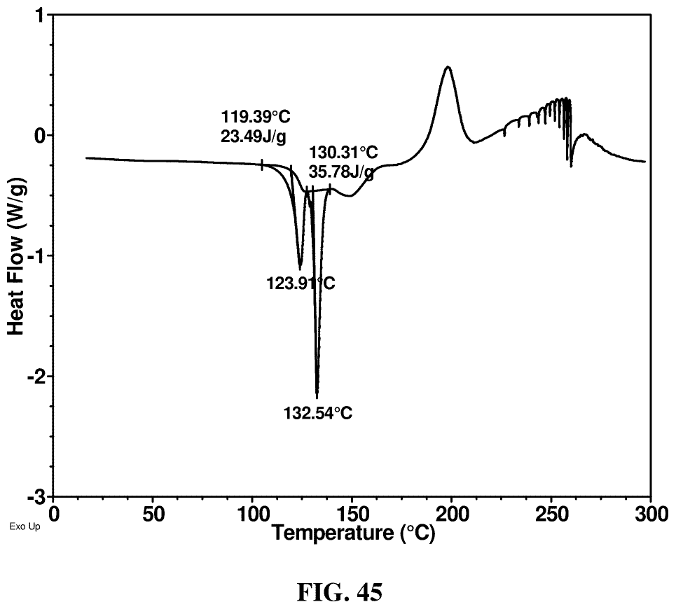

In some embodiments, the Compound 2, crystalline Form II is characterized by a DSC thermogram substantially as shown in FIG. 45.

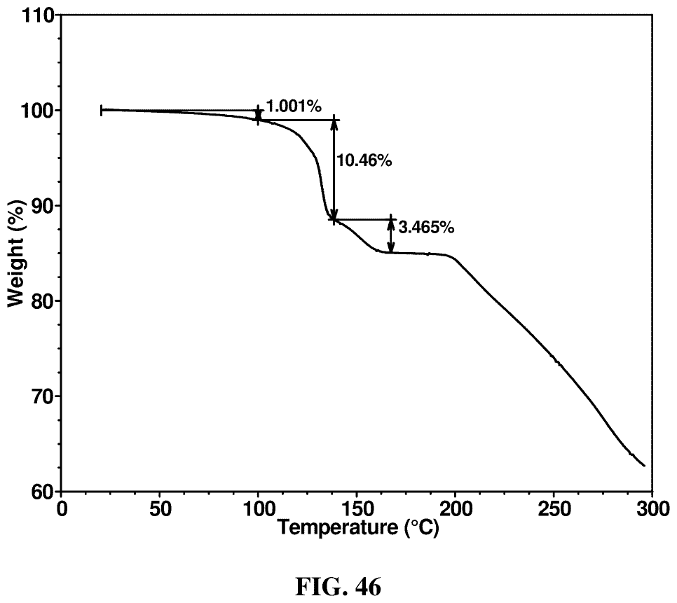

In some embodiments, the Compound 2, crystalline Form II is characterized by a TGA thermogram substantially as shown in FIG. 46.

In some embodiments, the crystalline form of Compound 2 provided herein is Compound 2, crystalline Form III.

In some embodiments, the Compound 2, crystalline Form III has at least one XRPD peak, in terms of 2-theta±0.2°, selected from 6.4°, 13.4°, 15.0°, 18.0°, 19.2°, 20.1°, 21.0°, 21.8°, and 22.1°.

In some embodiments, the Compound 2, crystalline Form III has at least two XRPD peaks, in terms of 2-theta±0.2°, selected from 6.4°, 13.4°, 15.0°, 18.0°, 19.2°, 20.1°, 21.0°, 21.8°, and 22.1°.

In some embodiments, the Compound 2, crystalline Form III has at least three XRPD peaks, in terms of 2-theta±0.2°, selected from 6.4°, 13.4°, 15.0°, 18.0°, 19.2°, 20.1°, 21.0°, 21.8°, and 22.1°.

In some embodiments, the Compound 2, crystalline Form III has at least four XRPD peaks, in terms of 2-theta±0.2°, selected from 6.4°, 13.4°, 15.0°, 18.0°, 19.2°, 20.1°, 21.0°, 21.8°, and 22.1°.

In some embodiments, the Compound 2, crystalline Form III has at least five XRPD peaks, in terms of 2-theta±0.2°, selected from 6.4°, 13.4°, 15.0°, 18.0°, 19.2°, 20.1°, 21.0°, 21.8°, and 22.1°.

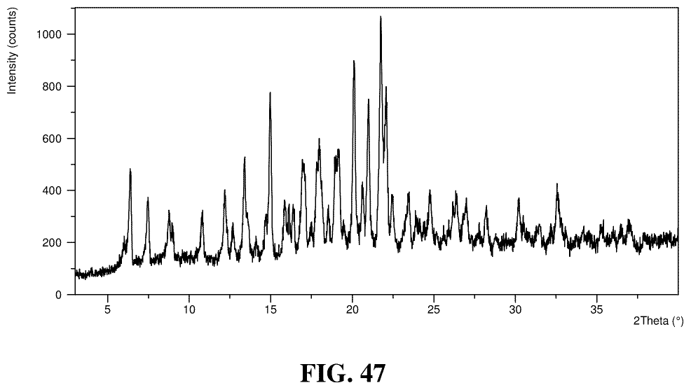

In some embodiments, the Compound 2, crystalline Form III is characterized by an XRPD pattern substantially as shown in FIG. 47.

In some embodiments, the Compound 2, crystalline Form III is characterized by a DSC thermogram having an endothermic event at about 109° C. In some embodiments, the Compound 2, crystalline Form III is characterized by a DSC thermogram having an endothermic transition at about 126° C. In some embodiments, the Compound 2, crystalline Form III is characterized by a DSC thermogram having an endothermic transition at about 141° C.

In some embodiments, the Compound 2, crystalline Form III is characterized by a DSC thermogram having at least one endothermic transition selected from about 109° C., about 126° C., and about 141° C. In some embodiments, the Compound 2, crystalline Form III is characterized by a DSC thermogram having at least two endothermic transitions selected from about 109° C., about 126° C., and about 141° C. In some embodiments, the Compound 2, crystalline Form III is characterized by a DSC thermogram having at least three endothermic transitions selected from about 109° C., about 126° C., and about 141° C.

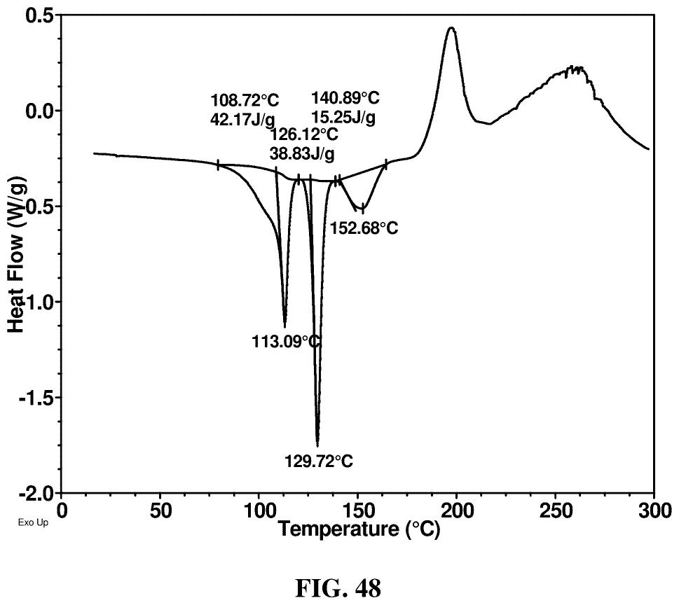

In some embodiments, the Compound 2, crystalline Form III is characterized by a DSC thermogram substantially as shown in FIG. 48.

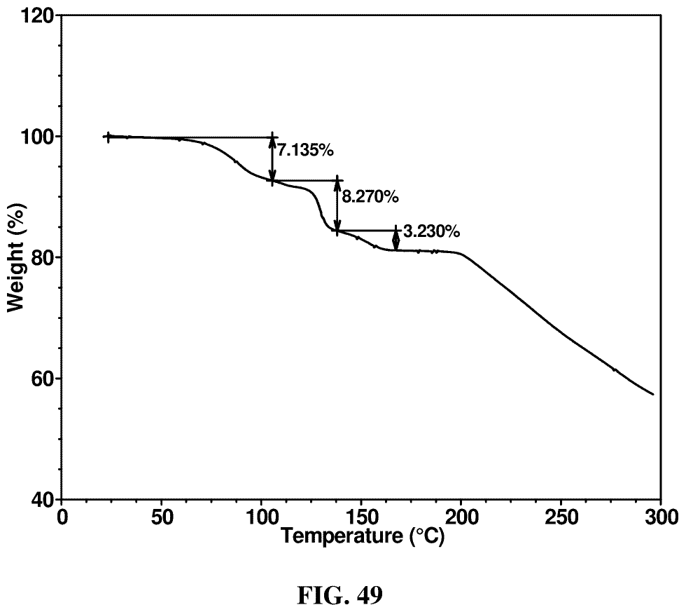

In some embodiments, the Compound 2, crystalline Form III is characterized by a TGA thermogram substantially as shown in FIG. 49.

### Compound 2, Solvate Forms

In some embodiments, the present application provides a solvate form of Compound 2.

In some embodiments, the solvate form of Compound 2 is selected from an isopropyl alcohol solvate form, a methyl tert-butyl ether solvate form, cyclopentyl methyl ether solvate form, and an acetone solvate form.

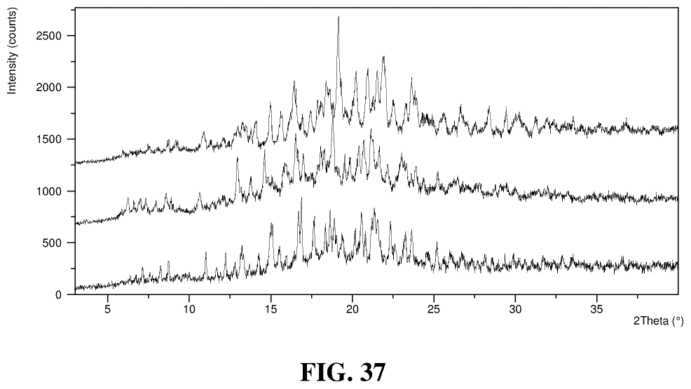

In some embodiments, the solvate form of Compound 2 is an isopropyl alcohol solvate form. In some embodiments, the Compound 2, isopropyl alcohol solvate form is characterized by an XRPD pattern substantially as shown in FIG. 37.

In some embodiments, the solvate form of Compound 2 is a methyl tert-butyl ether solvate form. In some embodiments, the Compound 2, methyl tert-butyl ether solvate form is characterized by an XRPD pattern substantially as shown in FIG. 37.

In some embodiments, the solvate form of Compound 2 is a cyclopentyl methyl ether solvate form. In some embodiments, the Compound 2, cyclopentyl methyl ether solvate form is characterized by an XRPD pattern substantially as shown in FIG. 37.

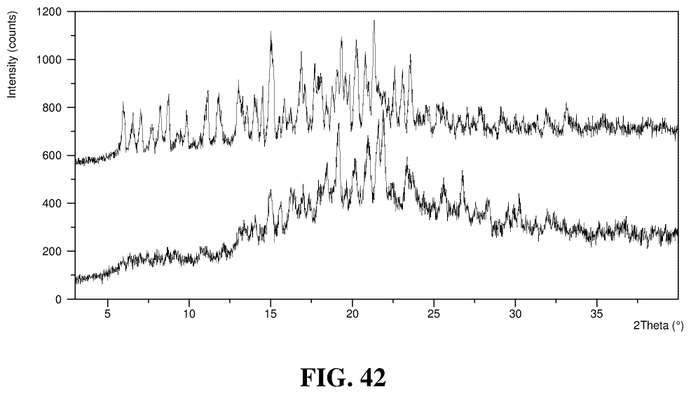

In some embodiments, the solvate form of Compound 2 is an acetone solvate form. In some embodiments, the Compound 2, acetone solvate form is characterized by an XRPD pattern substantially as shown in FIG. 42.

### Compound 2, Amorphous Form

In some embodiments, the present application provides an amorphous form of

### Compound 2.

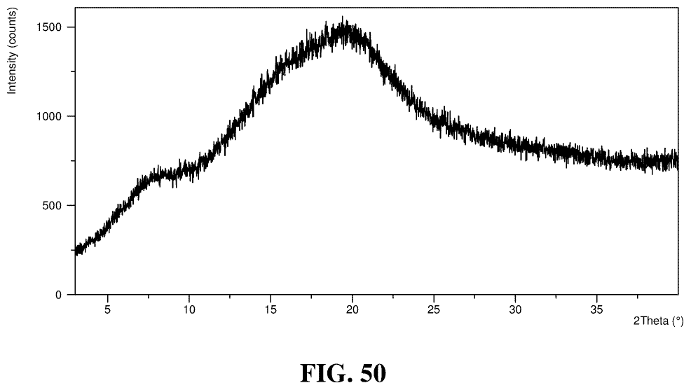

In some embodiments, the amorphous form of Compound 2 is characterized by an XRPD pattern substantially as shown in FIG. 50.

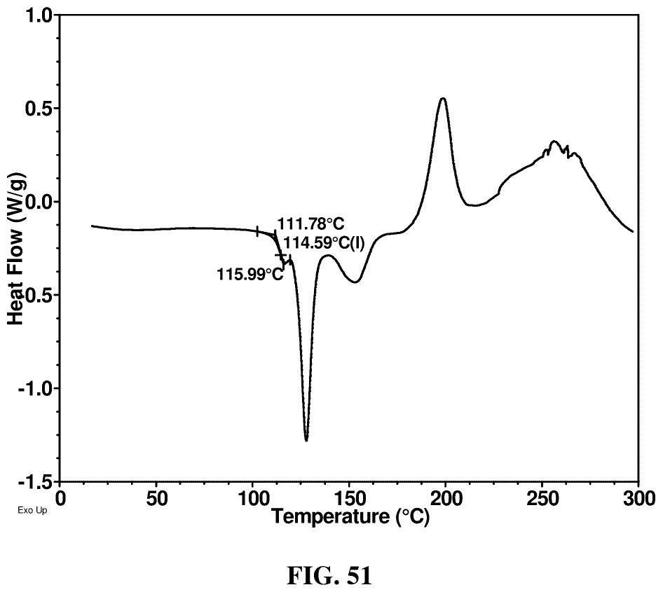

In some embodiments, the amorphous form of Compound 2 is characterized by a DSC thermogram substantially as shown in FIG. 51.

According to the present invention, a solid form provided herein (e.g., a crystalline form, solvate form, and/or co-crystal provided herein) can be useful in the synthesis and/or purification of a compound provided herein (e.g., Compound 1, or Compound 2). For example, a crystalline form of Compound 1 provided herein (e.g., crystalline Form II, or crystalline Form III) can be an intermediate in the synthesis of Compound 1. In addition, different crystalline forms and solvate form of Compound 1 may have different properties with respect to bioavailability, stability, purity, and/or manufacturability for medical or pharmaceutical uses. Variations in the crystal structure of a pharmaceutical drug substance or active ingredient may affect the dissolution rate (which may affect bioavailability, etc.), manufacturability (e.g., case of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, the crystalline forms and/or solvate forms of Compound 1 provided herein may provide advantages such as improving the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different crystalline forms and/or solvate forms of Compound 1, which may exhibit one or more of the favorable characteristics described above. The processes for the preparation of the crystalline forms and solvate forms described herein and characterization of these crystalline forms and solvate forms are described in detail below.

In some embodiments, the crystalline forms and solvate forms described herein, are purified or substantially isolated. By “substantially isolated” is meant that the crystalline form or solvate form is at least partially or substantially separated from the environment in which it was formed or detected. Partial separation can include, for example, a composition enriched in the crystalline form or solvate form of the invention. Substantial separation can include compositions containing at least about 50%, at least about 60%, at least about 70%, at least about 80%, at least about 90%, at least about 95%, at least about 97%, or at least about 99% by weight of the crystalline form or solvate form the invention. In some embodiments, the crystalline form or solvate form of the invention can be prepared with a purity of about 75% or more, 80% or more, 85% or more, 90% or more, 95% or more, 98% or more, or 99% or more.

Different crystalline forms and solvate forms can be identified by solid state characterization methods such as by X-ray powder diffraction (XRPD). Other characterization methods such as differential scanning calorimetry (DSC) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The description below is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art.

When trade names are used herein, it is intended to independently include the tradename product and the active pharmaceutical ingredient(s) of the tradename product.

As used herein and in the appended claims, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” refers to a salt of a compound that is pharmaceutically acceptable and that possesses (or can be converted to a form that possesses) the desired pharmacological activity of the parent compound.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like.

“Treatment” or “treating” is an approach for obtaining beneficial or desired results including clinical results. Beneficial or desired clinical results may include one or more of the following: a) inhibiting the disease or condition (i.e., decreasing one or more symptoms resulting from the disease or condition, and/or diminishing the extent of the disease or condition); b) slowing or arresting the development of one or more clinical symptoms associated with the disease or condition (i.e., stabilizing the disease or condition, preventing or delaying the worsening or progression of the disease or condition, and/or preventing or delaying the spread (i.e., metastasis) of the disease or condition); and/or c) relieving the disease, that is, causing the regression of clinical symptoms (i.e., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

As used herein, “delaying” development of a disease or condition means to defer, hinder, slow, retard, stabilize and/or postpone development of the disease or condition. This delay can be of varying lengths of time, depending on the history of the disease and/or subject being treated. As is evident to one skilled in the art, a sufficient or significant delay can, in effect, encompass prevention, in that the subject does not develop the disease or condition. For example, a method that “delays” development of AIDS is a method that reduces the probability of disease development in a given time frame and/or reduces extent of the disease in a given time frame, when compared to not using the method. Such comparisons may be based on clinical studies, using a statistically significant number of subjects. For example, the development of AIDS can be detected using known methods, such as confirming a subject's HIV+ status and assessing the subject's T-cell count or other indication of AIDS development, such as extreme fatigue, weight loss, persistent diarrhea, high fever, swollen lymph nodes in the neck, armpits or groin, or presence of an opportunistic condition that is known to be associated with AIDS (e.g., a condition that is generally not present in subjects with functioning immune systems but does occur in AIDS patients). Development may also refer to disease progression that may be initially undetectable and includes occurrence, recurrence and onset.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

The term “therapeutically effective amount” or “effective amount” of a solid form described herein means an amount sufficient to effect treatment when administered to a subject, to provide a therapeutic benefit such as amelioration of symptoms or slowing of disease progression. For example, a therapeutically effective amount may be an amount sufficient to improve a symptom of a Retroviridae viral infection, including but not limited to HIV infection. The therapeutically effective amount may vary depending on the subject, and the disease or condition being treated, the weight and age of the subject, the severity of the disease or condition, and the manner of administering, which can readily be determined by one of ordinary skill in the art.

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

“Diastereoisomers” are stereoisomers that have at least two asymmetric atoms, but which are not mirror-images of each other.

The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds and salts described herein contain one or more asymmetric centers and/or hindered rotation about a bond axis and may thus give rise to enantiomers, diastereomers, and other stereoisomeric forms that may be defined, in terms of absolute stereochemistry, as (R)- or (S)-. The present disclosure is meant to include all such possible isomers, including racemic mixtures, scalemic mixtures, diastereomeric mixtures, optically pure forms and intermediate mixtures. Optically active (R)- and (S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastercomerically or enantiomerically enriched form. Where chirality is not specified but is present, it is understood that the embodiment is directed to either the specific diastercomerically or enantiomerically enriched form; or a racemic or scalemic mixture of such compound(s).

One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC). Accordingly, the compound structure for Compound 1 provided above may also be named or identified as (2R,3S,5R)-5-(6-amino-2-fluoro-9H-purin-9-yl)-2-ethynyl-2-((2-phenylacetoxy)methyl)tetrahydrofuran-3-yl 2-phenylacetate.

In some embodiments, the current disclosure relates to the use of the crystalline forms and/or solvate forms of the invention in treating a Retroviridae viral infection including an infection caused by the HIV virus comprising administering a therapeutically effective amount of the crystalline form or solvate form to a subject in need thereof.

In some embodiments, the current disclosure relates to the use of the crystalline forms or solvate forms of the invention in treating a Retroviridae viral infection including an infection caused by the HIV virus comprising administering a therapeutically effective amount of the crystalline form or solvate form to a subject in need thereof.

It is a desirable goal to discover a compound, or crystalline form or solvate form thereof having a low EC₅₀. The EC₅₀ value refers to the concentration of a compound in an assay that achieves 50% of the maximum efficacy. A compound, crystalline form, or solvate form with a lower EC₅₀ achieves similar efficacy with lower compound, crystalline form, or solvate form concentration relative to a compound, crystalline form, or solvate form with a higher EC₅₀. Thus, a lower EC₅₀ is generally preferred for drug development.

It is a desirable goal to discover a compound, crystalline form, or solvate form that has good physical and/or chemical stability. An increase in overall stability of a compound, crystalline form, or solvate form can provide an increase in circulation time in the body. With less degradation, a stable compound, crystalline form, or solvate form can be administered in lower doses and still maintain efficacy. Also, with less degradation, there is less concern about by-products from degradation of a compound, crystalline form, or solvate form.

It is a desirable goal to discover a compound, crystalline form, or solvate form thereof that has improved pharmacokinetic and/or pharmacodynamic profiles and long half-life. It is advantageous for a drug to have a moderate or low clearance and a long half-life, as this can lead to a good bioavailability and high exposure in systemic exposure. Reducing the clearance and increasing half-life time of a compound, crystalline form, or solvate form could reduce the daily dose required for efficacy and therefore give a better efficacy and safety profile. Thus, improved pharmacokinetic and/or pharmacodynamic profiles and long half-life can provide for better patient compliance.

### Compositions and Kits

The compounds and solid forms provided herein (e.g., crystalline forms, solvate forms, and/or co-crystal forms provided herein), are usually administered in the form of pharmaceutical compositions. Thus, provided herein are also pharmaceutical compositions that comprise one or more of the solid forms provided herein and one or more pharmaceutically acceptable vehicles selected from carriers, adjuvants and excipients. The solid forms provided herein may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In one aspect, provided herein are pharmaceutical compositions comprising a solid forms provided herein (e.g., a crystalline form, a solvate form, and/or a co-crystal form provided herein), and a pharmaceutically acceptable excipient or carrier. In some embodiments, the pharmaceutical compositions comprise a therapeutically effective amount of a solid for provided herein, and a pharmaceutically acceptable excipient or carrier.

In some embodiments, the pharmaceutical compositions provided herein further comprise one or more (i.e., one, two, three, four; one or two; one to three; or one to four) additional therapeutic agents, or a pharmaceutically acceptable salt thereof. In some embodiments, the pharmaceutical compositions further comprise a therapeutically effective amount of the one or more (i.e., one, two, three, four; one or two; one to three; or one to four) additional therapeutic agents, or a pharmaceutically acceptable salt thereof.

In some embodiments, the one or more additional therapeutic agents include agents that are therapeutic for an HIV virus infection. In some embodiments, the one or more additional therapeutic agents is an anti-HIV agent. In some embodiments, the one or more additional therapeutic agents is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and any combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and any combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and any combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and any combinations thereof.

In some embodiments, the additional therapeutic agent or agents are chosen from dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, and tenofovir alafenamide hemifumarate, or a pharmaceutically acceptable salt thereof.

In some embodiments, the additional therapeutic agent or agents are chosen from dolutegravir, cabotegravir, darunavir, bictegravir, elsulfavirine, rilpivirine, and any combinations thereof, or a pharmaceutically acceptable salt thereof.

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, and dual-target HIV-1 reverse transcriptase/nucleocapsid protein 7 inhibitors.

In one embodiment, provided herein are pharmaceutical compositions comprising a therapeutically effective amount of a solid form provided herein, and a pharmaceutically acceptable excipient.

In some embodiments, the pharmaceutical compositions provided herein further comprise one, two, three, or four additional therapeutic agents.

In some embodiments, the pharmaceutical compositions provided herein further comprise one, two, three, or four additional therapeutic agents, wherein the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

In some embodiments, the pharmaceutical compositions provided herein further comprise one, two, three, or four additional therapeutic agents, wherein the additional therapeutic agents are selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, “antibody-like” therapeutic proteins, or any combinations thereof.

In some embodiments, the pharmaceutical compositions provided herein further comprise one, two, three, or four additional therapeutic agents, wherein the additional therapeutic agents are selected from the group consisting of dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, and tenofovir alafenamide hemifumarate, or a pharmaceutically acceptable salt thereof.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, the solid forms and pharmaceutical compositions disclosed herein are administered by subcutaneous injection.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the sterile injectable preparation disclosed herein may also be a sterile injectable solution or suspension prepared from a reconstituted lyophilized powder in a non-toxic parenterally acceptable diluent or solvent, such as a solution in 1,3-butane-diol. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found e.g., in Handbook of Pharmaceutical Excipients (eds. Rowe, Sheskey & Quinn), 6th edition 2009. Examples of solubilizing excipients in a parenteral formulation (e.g., an SC or IM formulation) include, but are not limited to, polysorbates (such as polysorbate 20 or 80) and poloxamers (such as poloxamer 338, 188, or 207).

In some embodiments, the solid forms and pharmaceutical compositions disclosed herein are administered with implants.

Oral administration may be another route for administration of the solid forms provided herein. Administration may be via, for example, capsule or enteric coated tablets. In making the pharmaceutical compositions that include at least one solid form provided herein, the active ingredient (such as a solid form provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one solid form described herein can be formulated so as to provide quick, sustained or delayed release of the active ingredient (such as a solid form provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the solid forms provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a solid form described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of the solid forms provided herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In one embodiment, provided herein are kits that comprise a solid form provided herein (e.g., a crystalline form, a solvate form, and/or a co-crystal form provided herein), and suitable packaging. In some embodiments, the kit further comprises instructions for use. In some embodiments, the kit comprises a solid form provided herein, and a label and/or instructions for use of the solid forms in the treatment of the indications, including the diseases or conditions, described herein.

In some embodiments, the kits further comprise one or more (i.e., one, two, three, four; one or two; one to three; or one to four) additional therapeutic agents, or a pharmaceutically acceptable salt thereof.

In one embodiment, provided herein are articles of manufacture that comprise a solid form described herein in a suitable container. In some embodiments, the container may be a vial, jar, ampoule, preloaded syringe, or intravenous bag.

### Methods

The methods provided herein may be applied to cell populations in vivo or ex vivo. “In vivo” means within a living individual, as within an animal or human. In this context, the methods provided herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. Exemplary tissue samples include tumors and biopsies thereof. In this context, the present disclosure may be used for a variety of purposes, including therapeutic and experimental purposes. For example, the present disclosure may be used ex vivo to determine the optimal schedule and/or dosing of administration of a compound as disclosed herein for a given cell type, individual, and other parameters. Information gleaned from such use may be used for experimental purposes or in the clinic to set protocols for in vivo treatment. Other ex vivo uses for which the present disclosure may be suited are described below or will become apparent to those skilled in the art. The selected solid forms may be further characterized to examine the safety or tolerance dosage in human or non-human subjects. Such properties may be examined using commonly known methods to those skilled in the art.

In one embodiment, the present disclosure provides a method of treating or preventing a human immunodeficiency virus (HIV) infection in a patient in need thereof comprising administering to the patient a therapeutically effective amount of a solid form provided herein (e.g., a crystalline form, a solvate form, and/or a co-crystal form provided herein), or a pharmaceutical composition provided herein.

In one embodiment, the present disclosure provides a method of treating a human immunodeficiency virus (HIV) infection in a heavily treatment-experienced patient, the method comprising administering to the patient a therapeutically effective amount of a solid form provided herein, or a pharmaceutical composition provided herein.

In some embodiments, the methods provided herein further comprise administering a therapeutically effective amount of one, two, three, or four additional therapeutic agents, or a pharmaceutically acceptable salt thereof.

In some embodiments, the one, two, three, or four additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

In some embodiments, the one, two, three, or four additional therapeutic agents are selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, and “antibody-like” therapeutic proteins, or any combinations thereof.

In some embodiments, the one, two, three, or four additional therapeutic agents are selected from the group consisting of dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, and tenofovir alafenamide hemifumarate, or a pharmaceutically acceptable salt thereof.

In some embodiments of the methods provided herein, the patient is a human.

In one embodiment, the present disclosure provides a therapeutically effective amount of a solid form provided herein, or a pharmaceutical composition provided herein for use in therapy.

In one embodiment, the present disclosure provides a solid form provided herein, or a pharmaceutical composition provided herein for use in a method of treating or preventing a human immunodeficiency virus (HIV) infection in a patient in need thereof, the method comprising administering to the patient a therapeutically effective amount of the solid form or the pharmaceutical composition.

In one embodiment, the present disclosure provides a solid form provided herein, or a pharmaceutical composition provided herein for use in a method of treating a human immunodeficiency virus (HIV) infection in a heavily treatment-experienced patient, the method comprising administering to the patient a therapeutically effective amount of the solid form or the pharmaceutical composition.

In some embodiments, the uses provided herein further comprise administering a therapeutically effective amount of one, two, three, or four additional therapeutic agents, or a pharmaceutically acceptable salt thereof.

In some embodiments of the uses provided herein, the one, two, three, or four additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

In some embodiments of the uses provided herein, the one, two, three, or four additional therapeutic agents are selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, and “antibody-like” therapeutic proteins, or any combinations thereof.

In some embodiments of the uses provided herein, the one, two, three, or four additional therapeutic agents are selected from the group consisting of dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, and tenofovir alafenamide hemifumarate, or a pharmaceutically acceptable salt thereof.

In some embodiments of the uses provided herein, the patient is a human.

### Administration

The solid forms of the present disclosure (e.g., crystalline forms, solvate forms, and/or co-crystals provided herein; also referred to herein as the active ingredients) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient.

A solid form of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the solid form is administered on a daily or intermittent schedule for the duration of the individual's life.

The specific dose level of a solid form of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a solid form provided herein, per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a solid form described herein administered per dose. The dosage or dosing frequency of a solid form of the present disclosure may be adjusted over the course of the treatment, based on the judgment of the administering physician.

The solid forms of the present disclosure may be administered to an individual (e.g., a human) in a therapeutically effective amount. In some embodiments, the solid form is administered once daily, once weekly, once monthly, once every two months, once every three months, or once every six months. In some embodiments, the solid form is administered once daily. In some embodiments, the solid form is administered once weekly. In some embodiments, the solid form is administered once monthly. In some embodiments, the solid form is administered once every two months. In some embodiments, the solid form is administered once every three months. In some embodiments, the solid form is administered once every six months.

The solid forms provided herein can be administered by any useful route and means, such as by oral or parenteral (e.g., intravenous) administration. Therapeutically effective amounts of the solid form may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the solid forms provided herein include from about 0.3 mg to about 30 mg per day, or from about 30 mg to about 300 mg per day, or from about 0.3 μg to about 30 mg per day, or from about 30 μg to about 300 μg per day.

A solid form of the present disclosure may be combined with one or more additional therapeutic agents in any dosage amount of the solid form of the present disclosure (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the solid form are about 50, 100, 125, 150, 175, 200, 225, 250, 275, or 300 mg per dose. Other therapeutically effective amounts of the solid forms are about 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, a therapeutically effective amount of the solid form is about 1 mg to about 1000 mg. In some embodiments, a therapeutically effective amount of the solid form is about 1 mg to about 900 mg. In some embodiments, a therapeutically effective amount of the solid form is about 1 mg to about 800 mg. In some embodiments, a therapeutically effective amount of the solid form is about 1 mg to about 700 mg. In some embodiments, a therapeutically effective amount of the solid form is about 1 mg to about 600 mg. In some embodiments, a therapeutically effective amount of the solid form is about 1 mg to about 500 mg. In some embodiments, a therapeutically effective amount of the solid form is about 1 mg to about 400 mg. In some embodiments, a therapeutically effective amount of the solid form is about 1 mg to about 300 mg. In some embodiments, a therapeutically effective amount of the solid form is about 1 mg to about 200 mg. In some embodiments, a therapeutically effective amount of the solid form is about 1 mg to about 100 mg. In some embodiments, a therapeutically effective amount of the solid form is about 1 mg to about 75 mg. In some embodiments, a therapeutically effective amount of the solid form is about 1 mg to about 50 mg. In some embodiments, a therapeutically effective amount of the solid form is about 1 mg to about 25 mg. In some embodiments, a therapeutically effective amount of the solid form is about 1 mg to about 20 mg. In some embodiments, a therapeutically effective amount of the solid form is about 1 mg to about 15 mg. In some embodiments, a therapeutically effective amount of the solid form is about 1 mg to about 10 mg. In some embodiments, a therapeutically effective amount of the solid form is about 1 mg to about 5 mg.

In some embodiments, a therapeutically effective amount of the solid form is about 50 mg, about 75 mg, about 100 mg, about 125 mg, about 150 mg, about 175 mg, about 200 mg, about 225 mg, about 275 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, or about 1050 mg. In some embodiments, a therapeutically effective amount of the solid form is about 5 mg. In some embodiments, a therapeutically effective amount of the solid form is about 100 mg. In some embodiments, a therapeutically effective amount of the solid form is about 150 mg. In some embodiments, a therapeutically effective amount of the solid form is about 200 mg. In some embodiments, a therapeutically effective amount of the solid form is about 250 mg. In some embodiments, a therapeutically effective amount of the solid form is about 300 mg. In some embodiments, a therapeutically effective amount of the solid form is about 350 mg. In some embodiments, a therapeutically effective amount of the solid form is about 400 mg. In some embodiments, a therapeutically effective amount of the solid form is about 450 mg. In some embodiments, a therapeutically effective amount of the solid form is about 500 mg. In some embodiments, a therapeutically effective amount of the solid form is about 550 mg. In some embodiments, a therapeutically effective amount of the solid form is about 600 mg. In some embodiments, a therapeutically effective amount of the solid form is about 650 mg. In some embodiments, a therapeutically effective amount of the solid form is about 700 mg. In some embodiments, a therapeutically effective amount of the solid form is about 750 mg. In some embodiments, a therapeutically effective amount of the solid form is about 800 mg. In some embodiments, a therapeutically effective amount of the solid form is about 850 mg. In some embodiments, a therapeutically effective amount of the solid form is about 900 mg. In some embodiments, a therapeutically effective amount of the solid form is about 950 mg. In some embodiments, a therapeutically effective amount of the solid form is about 1000 mg. In some embodiments, a therapeutically effective amount of the solid form is about 1050 mg.

When administered orally, the total weekly dosage for a human subject may be between about 1 mg and 1,000 mg/week, between about 10-500 mg/week, between about 50-300 mg/week, between about 75-200 mg/week, or between about 100-150 mg/week. In some embodiments, the total weekly dosage for a human subject may be about 100, 200, 300, 400, 500, 600, 700, 800, 900, or 1000 mg/week administered in a single dose. In some embodiments, the total weekly dosage for a human subject of a solid form may be about 100 mg administered in a single dose. In some embodiments, the total weekly dosage for a human subject of a solid form may be about 150 mg administered in a single dose. In some embodiments, the total weekly dosage for a human subject of a solid form may be about 200 mg administered in a single dose. In some embodiments, the total weekly dosage for a human subject of a solid form may be about 250 mg administered in a single dose. In some embodiments, the total weckly dosage for a human subject of a solid form may be about 300 mg administered in a single dose. In some embodiments, the total weekly dosage for a human subject of a solid form may be about 350 mg administered in a single dose. In some embodiments, the total weekly dosage for a human subject of a solid form may be about 400 mg administered in a single dose. In some embodiments, the total weekly dosage for a human subject of a solid form may be about 450 mg administered in a single dose. In some embodiments, the total weekly dosage for a human subject of a solid form may be about 500 mg administered in a single dose.

When administered orally, the total monthly dosage for a human subject of a solid form may be between about 500 mg and 1,000 mg/month, between about 600-900 mg/month, or between about 700-800 mg/month. In some embodiments, the total weekly dosage for a human subject of a solid form may be about 100, 200, 300, 400, 500, 600, 700, 800, 900, or 1000 mg/week administered in a single dose. In some embodiments, the total monthly dosage for a human subject of a solid form may be about 500 mg administered in a single dose. In some embodiments, the total monthly dosage for a human subject may be about 550 mg administered in a single dose. In some embodiments, the total monthly dosage for a human subject of a solid form may be about 600 mg administered in a single dose. In some embodiments, the total monthly dosage for a human subject of a solid form may be about 650 mg administered in a single dose. In some embodiments, the total monthly dosage for a human subject of a solid form may be about 700 mg administered in a single dose. In some embodiments, the total monthly dosage for a human subject of a solid form may be about 750 mg administered in a single dose. In some embodiments, the total monthly dosage for a human subject of a solid form may be about 800 mg administered in a single dose. In some embodiments, the total monthly dosage for a human subject of a solid form may be about 850 mg administered in a single dose. In some embodiments, the total monthly dosage for a human subject of a solid form may be about 900 mg administered in a single dose. In some embodiments, the total monthly dosage for a human subject of a solid form may be about 950 mg administered in a single dose. In some embodiments, the total monthly dosage for a human subject of a solid form may be about 1000 mg administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a solid form provided herein is administered once daily in a method disclosed herein. In some embodiments, a solid form provided herein is administered twice daily in a method disclosed herein.

In some embodiments, a solid form provided herein is administered once daily in a method disclosed herein. In some embodiments, a solid form provided herein is administered once weekly in a method disclosed herein. In some embodiments, a solid form provided herein is administered once monthly in a method disclosed herein. In some embodiments, a solid form provided herein is administered once every two months in a method disclosed herein. In some embodiments, a solid form provided herein is administered once every three months in a method disclosed herein. In some embodiments, a solid form provided herein is administered once every six months in a method disclosed herein.

In some embodiments, a solid form provided herein is administered orally in a single dose of about 100 mg once weekly. In some embodiments, a solid form provided herein is administered orally in a single dose of about 150 mg once weekly. In some embodiments, a solid form provided herein is administered orally in a single dose of about 200 mg once weekly. In some embodiments, a solid form provided herein is administered orally in a single dose of about 250 mg once weekly. In some embodiments, a solid form provided herein is administered orally in a single dose of about 300 mg once weekly. In some embodiments, a solid form provided herein is administered orally in a single dose of about 350 mg once weekly. In some embodiments, a solid form provided herein is administered orally in a single dose of about 400 mg once weekly. In some embodiments, a solid form provided herein is administered orally in a single dose of about 450 mg once weekly. In some embodiments, a solid form provided herein is administered orally in a single dose of about 500 mg once weekly.

In some embodiments, a solid form provided herein is administered orally in a single dose of about 500 mg once monthly. In some embodiments, a solid form provided herein is administered orally in a single dose of about 550 mg once monthly. In some embodiments, a solid form provided herein is administered orally in a single dose of about 600 mg once monthly. In some embodiments, a solid form provided herein is administered orally in a single dose of about 650 mg once monthly. In some embodiments, a solid form provided herein is administered orally in a single dose of about 700 mg once monthly. In some embodiments, a solid form provided herein is administered orally in a single dose of about 750 mg once monthly. In some embodiments, a solid form provided herein is administered orally in a single dose of about 800 mg once monthly. In some embodiments, a solid form provided herein is administered orally in a single dose of about 850 mg once monthly. In some embodiments, a solid form provided herein is administered orally in a single dose of about 900 mg once monthly. In some embodiments, a solid form provided herein is administered orally in a single dose of about 950 mg once monthly. In some embodiments, a solid form provided herein is administered orally in a single dose of about 1000 mg once monthly.

The frequency of dosage of the solid form of the present disclosure will be determined by the needs of the individual patient and can be, for example, once per day, once per week, once per month, once per every two months, once per every three months, or once per every six months. Administration of the solid form continues for as long as necessary to treat the Retroviridae infection, including an HIV infection, or any other indication described herein. For example, a solid form can be administered to a human suffering from a Retroviridae infection, including an HIV infection, for the duration of the human's life.

Administration can be intermittent, with a period of several or more days during which a patient receives a daily dose of the solid form of the present disclosure followed by a period of several or more days during which a patient does not receive a daily dose of the solid form. For example, a patient can receive a dose of the solid form every other day, or three times per week. Again by way of example, a patient can receive a dose of the solid form each day for a period of from 1 to 14 days, followed by a period of 7 to 21 days during which the patient does not receive a dose of the solid form followed by a subsequent period (e.g., from 1 to 14 days) during which the patient again receives a daily dose of the solid form. Alternating periods of administration of the solid form followed by non-administration of the solid form can be repeated as clinically required to treat the patient.

The solid forms of the present disclosure, or the pharmaceutical compositions of the present disclosure may be administered once, twice, three, or four times daily, using any suitable mode described above. Also, administration or treatment with the solid forms may be continued for a number of days; for example, commonly treatment would continue for at least 7 days, 14 days, or 28 days, for one cycle of treatment. Treatment cycles are well known for Retroviridae infections, including an HIV infection. In some embodiments, treatment cycles are frequently alternated with resting periods of about 1 to 28 days, commonly about 7 days or about 14 days, between cycles. The treatment cycles, in other embodiments, may also be continuous.

### Combination Therapy

Patients being treated by administration of the solid forms provided herein (e.g., crystalline forms, solvate forms, and/or co-crystal forms provided herein) often exhibit diseases or conditions that benefit from treatment with other therapeutic agents, including agents that are therapeutic for Retroviridae infections, including an HIV infection. In some embodiments, the other therapeutic agent is an agent that is therapeutic for an HIV infection. Thus, one aspect of the disclosure is a method of treating an HIV infection comprising administering a solid form of the present disclosure in combination with one or more compounds useful for the treatment of an HIV infection to a subject, particularly a human subject, in need thereof.

In some embodiments, a solid form of the present disclosure is combined with one, two, three, four or more additional therapeutic agents. In some embodiments, a solid form of the present disclosure is combined with two additional therapeutic agents. In some embodiments, a solid form of the present disclosure is combined with three additional therapeutic agents. In some embodiments, a solid form of the present disclosure is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a solid form of the present disclosure is combined with one or more additional therapeutic agents as described herein, the components of the composition are administered as a simultaneous or sequential regimen. When administered sequentially, the combination may be administered in two or more administrations.

In some embodiments, a solid form of the present disclosure is combined with one or more additional therapeutic agents in a unitary dosage form for simultaneous administration to a patient, for example as a solid dosage form for oral administration.

In some embodiments, a solid form of the present disclosure is co-administered with one or more additional therapeutic agents.

Co-administration includes administration of unit dosages of the solid forms provided herein before or after administration of unit dosages of one or more additional therapeutic agents. The solid forms provided herein may be administered within seconds, minutes, or hours of the administration of one or more additional therapeutic agents. For example, in some embodiments, a unit dose of a solid form provided herein is administered first, followed within seconds or minutes by administration of a unit dose of one or more additional therapeutic agents. Alternatively, in other embodiments, a unit dose of one or more additional therapeutic agents is administered first, followed by administration of a unit dose of a solid form provided herein within seconds or minutes. In some embodiments, a unit dose of a solid form provided herein is administered first, followed, after a period of hours (i.e., 1-12 hours), by administration of a unit dose of one or more additional therapeutic agents. In other embodiments, a unit dose of one or more additional therapeutic agents is administered first, followed, after a period of hours (i.e., 1-12 hours), by administration of a unit dose of a solid form provided herein.

In some embodiments, a solid form provided herein (e.g., a crystalline form, solvate form, and/or co-crystal form provided herein), is formulated as a tablet, which may optionally contain one or more other compounds useful for treating the disease being treated. In certain embodiments, the tablet can contain another active ingredient for treating a Retroviridae infection, including an HIV infection. In some embodiments, such tablets are suitable for once daily dosing. In some embodiments, such tablets are suitable for once weekly dosing. In some embodiments, such tablets are suitable for once monthly dosing. In some embodiments, such tablets are suitable for once every two months dosing. In some embodiments, such tablets are suitable for once every three months dosing. In some embodiments, such tablets are suitable for once every six months dosing.

Also provided herein are methods of treatment in which a solid form is given to a patient in combination with one or more additional therapeutic agents or therapy. In some embodiments, the total daily dosage of a solid form may be about 1 to about 500 mg administered in a single dose for a human subject.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents any are chosen from dolutegravir, cabotegravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911. Additional examples of HIV protease inhibitors are described, e.g., in U.S. Pat. No. 10,294,234, and U.S. Patent Application Publication Nos. US 2020030327 and US 2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), elsulfavirine (long acting injectable nanosuspension, HIV infection), and elsulfavirine (VM-1500). Additional non-limiting examples of non-nucleoside or non-nucleotide inhibitors of reverse transcriptase include the compounds disclosed in U.S. Pat. No. 10,548,898.

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017. Additional non-limiting examples of HIV integrase inhibitors include the compounds disclosed in U.S. Pat. No. 11,084,832.

Examples of HIV non-catalytic site, or allosteric, integrase inhibitors (NCINI) include, but are not limited to, CX-05045, CX-05168, and CX-14442.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, Cl3hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

Additional examples of HIV capsid inhibitors include, but are not limited to, those described in U.S. Patent Application Publication Nos. US2014221356 and US2016016973.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18 (11): 671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript IE (RAETIE; ULBP4); retinoic acid early transcript 1G (RAETIG; ULBP5); retinoic acid early transcript IL (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor DI (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, arc combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor DI (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18 (11): 671-688.

In some embodiments, the one or more immune checkpoint inhibitors comprises a proteinaccous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Genc ID: 8793), TNFRSF11A (CD265, RANK, NCBI Gene ID: 8792), TNFRSF11B (NCBI Genc ID: 4982), TNFRSF12A (CD266, NCBI Gene ID: 51330), TNFRSF13B (CD267, NCBI Genc ID: 23495), TNFRSF13C (CD268, NCBI Gene ID: 115650), TNFRSF16 (NGFR, CD271, NCBI Genc ID: 4804), TNFRSF17 (BCMA, CD269, NCBI Gene ID: 608), TNFRSF18 (GITR, CD357, NCBI Genc ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi-and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRIKE) include, but are not limited to, OXS-3550, HIV-TriKE, and CD16-IL-15-B7H3 TriKe.

Indoleamine-pyrrole-2,3-dioxygenase (IDO1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO—C101, IL-15 Synthorin (pegylated Il-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

alpha-4/beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492 (7427): 118-22 (2012), Horwitz et al., Proc Natl Acad Sci USA, 110 (41): 16538-43 (2013), Scheid et al., Science, 333: 1633-1637 (2011), Scheid et al., Nature, 458:636-640 (2009), Eroshkin et al, Nucleic Acids Res., 42 (Database issue): DI 133-9 (2014), Mascola et al., Immunol Rev., 254 (1): 225-44 (2013), such as 2F5, 4E10, M66.6, CAP206-CH12, 10E81 (all of which bind the MPER of gp41); PG9, PG16, CH01-04 (all of which bind VIV2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173 (7): 1783-1795; Sajadi et al., J Infect Dis. (2016) 213 (1): 156-64; Klein et al., Nature, 492 (7427): 118-22 (2012), Horwitz et al., Proc Natl Acad Sci US A, 110 (41): 16538-43 (2013), Scheid et al., Science, 333:1633-1637 (2011), Scheid et al., Nature, 458:636-640 (2009), Eroshkin et al., Nucleic Acids Res., 42 (Database issue): DI 133-9 (2014), Mascola et al., Immunol Rev., 254 (1): 225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, DID2, 10-1074-LS, Cl3hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, cCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US20140221356 (Gilead Sciences), US20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp 140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp 120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PTI, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, Pre VaxTat, AE-H, MYM-V101, CombiHIVvac, ADVAX, MYM-V201, MVA-CMDR, MagaVax, DNA-Ad5 gag/pol/ncf/ncv (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp 160 HIV vaccine, RNActive HIV vaccine, SCB-703, Tat Oyi vaccine, TBC-M4, UBI HIV gp120, Vacc-4x+romidepsin, variant gp 120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; COD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvScq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a solid form disclosed herein is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDHI (LV-800, HIV infection), AGT103-T, and SupTI cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp 120 or a portion thereof, a CD4 binding site on gp120, the CD4-induced binding site on gp 120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A solid as disclosed herein (e.g., a crystalline form, solvate form, and/or co-crystal form disclosed herein) may be combined with one, two, three, or four additional therapeutic agents in any dosage amount of the solid form (e.g., from 1 mg to 500 mg of the solid form).

In one embodiment, kits comprising a solid form disclosed herein in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a solid form disclosed herein and an HIV nucleoside or nucleotide inhibitor of reverse transcriptase. In a specific embodiment, the kit includes a solid form disclosed herein and an HIV nucleoside or nucleotide inhibitor of reverse transcriptase and an HIV non-nucleoside inhibitor of reverse transcriptase. In another specific embodiment, the kit includes a solid form disclosed herein and an HIV nucleoside or nucleotide inhibitor of reverse transcriptase, and an HIV protease inhibiting compound. In an additional embodiment, the kit includes a solid form disclosed herein, an HIV nucleoside or nucleotide inhibitor of reverse transcriptase, an HIV non-nucleoside inhibitor of reverse transcriptase, and a pharmacokinetic enhancer. In certain embodiments, the kit includes a solid form disclosed herein, at least one HIV nucleoside inhibitor of reverse transcriptase, an integrase inhibitor, and a pharmacokinetic enhancer. In another embodiment, the kit includes a solid form disclosed herein, and two HIV nucleoside or nucleotide inhibitors of reverse transcriptase. In a specific embodiment, the kit includes a solid form disclosed herein, an HIV nucleoside or nucleotide inhibitor of reverse transcriptase and an HIV capsid inhibitor. In a specific embodiment, the kit includes a solid form disclosed herein, an HIV nucleoside inhibitor of reverse transcriptase and an HIV capsid inhibitor. In a specific embodiment, the kit includes a solid form disclosed herein, and an HIV capsid inhibitor. In a specific embodiment, the kit includes a solid form disclosed herein, and one, two, three or four HIV bNAbs. In a specific embodiment, the kit includes a solid form disclosed herein, one, two, three or four HIV bNAbs and an HIV capsid inhibitor. In a specific embodiment, the kit includes a solid form disclosed herein, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, doravirine, raltegravir, and long acting dolutegravir.

### Kits and Articles of Manufacture

The present disclosure relates to a kit comprising a solid form disclosed herein (e.g., a crystalline form, solvate form, and/or co-crystal form disclosed herein). In some embodiments, the kit may comprise one or more additional therapeutic agents as described hereinbefore. The kit may further comprise instructions for use, e.g., for use in inhibiting an HIV reverse transcriptase, such as for use in treating an HIV infection or AIDS or as a research tool. The instructions for use are generally written instructions, although electronic storage media (e.g., magnetic diskette or optical disk) containing instructions are also acceptable.

The present disclosure also relates to a pharmaceutical kit comprising one or more containers comprising a solid form disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

In some embodiments, the present disclosure also relates to a pharmaceutical kit comprising one or more containers comprising a solid form disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

Also disclosed are articles of manufacture comprising a unit dosage of a crystalline form or solvate form of Compound 1 disclosed herein in suitable packaging for use in the methods described herein. Suitable packaging is known in the art and includes, for example, vials, vessels, ampules, bottles, jars, flexible packaging and the like. An article of manufacture may further be sterilized and/or sealed.

The following abbreviations may be used herein:
° C. Degree Celsius
DIPE Diisopropyl ether
DSC Differential scanning calorimetry
DVS Dynamic Vapor Sorption
eq. or equiv Equivalent
M Molar
MDSC Modulated differential scanning calorimetry
NMR Nuclear magnetic resonance
SCXRD Single crystal x-ray diffraction
Tg Glass transition temperature
TGA Thermogravimetric analysis
XRPD or XRD X-Ray powder diffraction
DCM Dichloromethane
EtOAc Ethyl acetate
EtOH Ethanol
IPA Isopropanol
IPAc Isopropyl acetate
IPE Diisopropyl ether
MeCN Acetonitrile
MEK Methyl ethyl ketone
MeOH Methanol
MIBK Methyl isobutyl ketone
MTBE Methyl tert-butyl ether
THF Tetrahydrofuran
BuOAc n-Butyl acetate
DBE Di-n-butyl ether
MeOAc Methyl acetate

### EXAMPLES

### General Materials and Methods

X-ray powder diffraction (XRPD) analysis was conducted on a diffractometer (PANalytical XPERT-PRO, PANalytical B. V., Almelo, Netherlands) using copper radiation (Cu Kα, λ=1.541874). Samples were spread evenly on a zero-background sample plate. The generator was operated at a voltage of 45 kV and amperage of 40 mA. Slits were Soller 0.02 rad, antiscatter 1.0°, and divergence. Scans were performed from 2 to 40° 2θ with a 0.0167 step size. Data analysis was performed using X'Pert Viewer V1.9a (PANalytical B.V., Almelo, Netherlands).

Differential Scanning calorimetry (DSC) and Modulated Differential Scanning calorimetry (MDSC) were run on Model Q2000 (TA Instruments, New Castle, DE). About 1-5 mg of material was loaded into a Tzero standard aluminum pan with manually punctured a pinhole on the lid. By default, the sample and reference pans were heated from 20 to 300° C. under nitrogen purge of 50 mL/min. DSC was run at a heating rate of 10° C./min; while MDSC was run at a heating rate of 5° C./min from with modulation of ±1.00° C. every 60 s. Data analysis was completed using Universal Analysis 2000 Version 4.5A (TA Instruments, New Castle, DE).

Thermogravimetric Analysis (TGA) was used to evaluate sample weight loss as a function of temperature on either Model Q5000 or Q500 (TA Instruments, New Castle, DE). About 1 to 5 mg of material was loaded onto a weigh pan and the sample was heated from ambient temperature to 300° C. or above at a rate of 10° C./min. The sample pan was under nitrogen purge at 40 mL/min. Data analysis was completed using Universal Analysis 2000 Version 4.5A (TA Instruments, New Castle, DE).

Dynamic Vapor Sorption (DVS) was used to study hygroscopicity on Model Q5000 SA (TA Instruments, New Castle, DE). A sample (1-10 mg) was placed in an aluminum pan and loaded on the sample side of the twin pan balance. The water sorption and desorption were studied as a function of relative humidity (RH) at 25° C. in 10% RH increments from 0% RH to 90% RH and then back to 0%. Each relative humidity increment had an equilibration time of 120 minutes unless weight change % was less than 0.002% in 20 minutes. Data analysis was performed using Universal Analysis 2000 Version 4.7A (TA Instruments, New Castle, DE).

¹⁹F Nuclear Magnetic Resonance spectra were collected on a Varian 400-MR 400 MHz instrument with 7620AS sample changer. Some of the parameters were adjusted as follows: number of scans: 1; spin: 0 (off); steady state scans: 0 (off). Samples were prepared as a solution of 20 mg in 0.75 mL of dimethyl sulfoxide-d6. The time from solvent addition to end of data acquisition should be ≤5 minutes. Off-line analysis was conducted using MNova software.

### Example 1. Preparation of Compound 1, Crystalline Form I

About 50 mg of Compound 1, Material A was stirred in 0.1 mL of MeCN at about 22° C. over a period of 20 hours. XRPD pattern of the wet cake, designated as MeCN solvate, is shown in FIG. 2. After drying at 50° C. under vacuum, it became Compound 1, crystalline Form I.

Antisolvents were explored to improve the yield of Compound 1, MeCN solvate. While both IPE and DBE were able to reduce the solubility of Compound 1, MTBE was found to increase the solubility, and toluene (1:1 mixture with MeCN) resulted in a different solvate that became Compound 1, Material A upon drying.

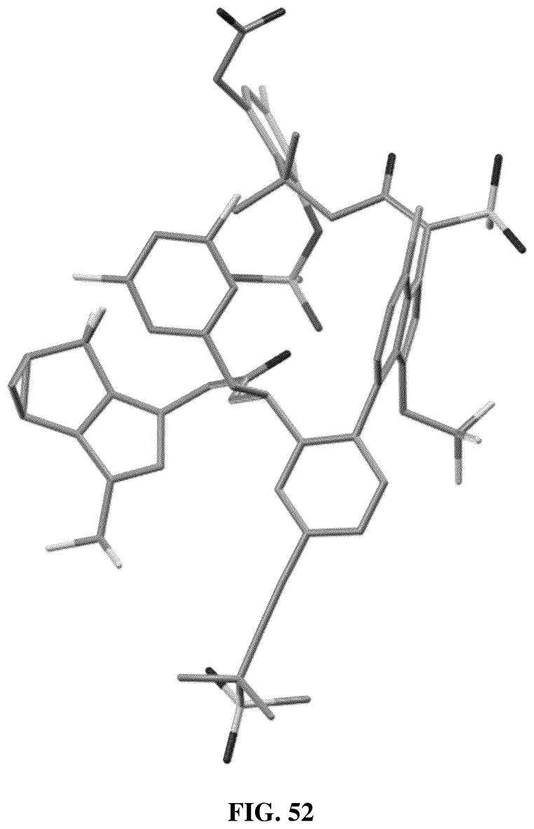

Single crystals of Compound 1, MeCN solvate were prepared by holding a solution of 50 mg of Compound 1 in 0.2 mL of MeCN and 0.1 mL of IPE at 40° C. in the presence of Compound 1, crystalline Form I seeds for a week to form a suspension, which was cooled to about 22° C. and submitted for SCXRD analysis. The data showed 2.767 equivalents of MeCN, and 0.289 equivalents of water. Although water was not deliberately added in the solution, it may have been introduced into the system by either solids or solvents. The data confirmed that the crystal consisted of the major atropisomer at the biaryl rotation axis, as shown in FIG. 52. Additional results of the SCXRD analysis of Compound 1, MeCN solvate are provided in Table 1.

TABLE 1

| Temp. | 100.0 K |
|---|---|
| Crystal system | Orthorhombic |
| Space group | P2₁2₁2₁ |
| Unit cell dimensions | a = 14.8328(2) Å |
| | b = 15.0500(2) Å |
| | c = 29.3514(5) Å |
| | α = 90° |
| | β = 90° |
| | γ = 90° |
| Z | 4 |
| Density (calculated) | 1.434 g/cm3 |
| Goodness-of-fit on F2 | 1.084 |
| Crystal composition | Compound |
| | 1:water:MeCN = |
| | 1:0.289:2.767 |

Compound 1, crystalline Form I was also obtained after drying at 50 to 75° C. from other solvates (as precursors) that had been obtained by stirring Compound 1 in the corresponding solvents (preferably with the seeds of Compound 1, crystalline Form I) with an appropriate antisolvent such as diisopropyl ether, n-butyl ether, or heptanes, if necessary. The XRPD patterns of the Compound 1 solvate precursors are shown in FIG. 3.

Compound 1, crystalline Form I was also obtained from amorphous Compound 1. 5 g of amorphous Compound 1 was stirred in 5 mL MeCN in a vial at about 22° C. While most of the solid dissolved, some sticky substance remained at the bottom of the vial. The sample was sonicated for a few minutes, and a thick slurry was obtained. The slurry was diluted with 5 mL of MeCN and 5 mL of DIPE. After stirring for 30 min at 22° C., the slurry was then cooled to 3° C. over 30 min. The slurry was further diluted with 15 mL of DIPE. After stirring for an additional 30 min, 10 mL heptanes were added. The mixture became sticky after awhile at 3° C. The mixture was heated back to 22° C. and was sonicated to become a slurry. The slurry was stirred for two days at 22° C. and was filtered, washed with 10 mL of DIPE, and then 10 mL of heptanes. The wet cake was dried in the vacuum oven at 50° C. overnight to provide Compound 1, crystalline Form I, which was confirmed by XRPD analysis.

A representative XRPD pattern of Compound 1, crystalline Form I (after drying the MeCN solvate at 50° C. under vacuum) is shown in FIG. 4 and is characterized by sharp reflections, indicating crystallinity. An XRPD peak listing is provided in Table 1-A.

TABLE 1-A

| 2-Theta (°) | Relative Intensity (%) |
|---|---|
| 6.2 | 100 |
| 6.6 | 14 |
| 8.7 | 26 |
| 10.5 | 18 |
| 11.0 | 4 |
| 12.4 | 23 |
| 12.6 | 17 |
| 13.2 | 8 |
| 13.8 | 57 |
| 16.1 | 20 |
| 16.5 | 12 |
| 17.0 | 8 |
| 17.5 | 14 |
| 17.7 | 16 |
| 18.0 | 7 |
| 18.6 | 15 |
| 19.8 | 14 |
| 20.7 | 19 |
| 21.5 | 11 |
| 22.3 | 14 |
| 23.1 | 28 |
| 23.4 | 11 |
| 24.2 | 8 |
| 25.7 | 36 |
| 26.3 | 20 |
| 29.3 | 5 |
| 30.2 | 10 |

The DSC thermogram of Compound 1, crystalline Form I indicated an endotherm event with onset at about 202° C., attributed to melting, and is shown in FIG. 5. The TGA thermogram, shown in FIG. 6, showed a weight loss of about 0.04% (from 25 to 150° C.), indicating that the solid is not solvated. DVS analysis indicated that the form is hygroscopic with about 4% water uptake from 0 to 90% RH at 25° C., as shown in FIG. 7.

### Example 2. Preparation of Compound 1, Crystalline Form II

About 50 mg of Compound 1, crystalline Form I was stirred in about 0.2 mL of DCM at about 22° C. over a period of 2 weeks. XRPD pattern (see FIG. 8) of the wet cake showed that it was a new solvate (comparing with Compound 1, DCM Solvate 1), designated as Compound 1, DCM Solvate 2. The solid became Compound 1, crystalline Form II after drying at about 50° C. under vacuum.

Compound 1, crystalline Form II was also be obtained as follows. About 50 mg of Compound 1, crystalline Form I was stirred in about 0.2 mL of BuOAc at about 22° C. in the presence of combined seeds of Compound 1, Forms I, II, III, and Compound 1, Material A. After two days, XRPD pattern (FIG. 8) of the wet cake showed that it was a new solvate (comparing with Compound 1, BuOAc Solvate 1), designated as Compound 1 BuOAc solvate 2. The solid became Compound 1, crystalline Form II after drying at about 50° C. under vacuum.

More experiments indicated that Compound 1, crystalline Form II may be obtained after drying at 50 to 75° C. from other solvates (as precursors) that had been obtained by stirring Compound 1 in the corresponding solvents (preferably with the seeds of Compound 1, crystalline Form II) with appropriate antisolvents such as diisopropyl ether, n-butyl ether, or heptanes, if necessary. The XRPD patterns of these solvate precursors are shown in FIG. 8.

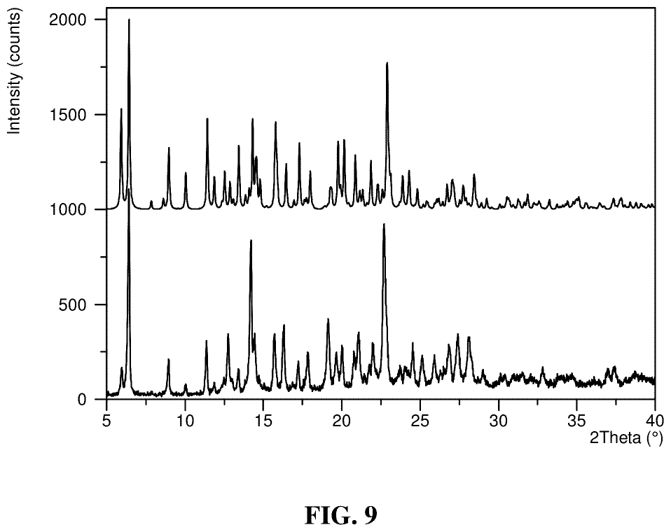

During experiments to acquire SCXRD of Compound 1, DCM solvate 2, SCXRD data of Compound 1, crystalline Form II was obtained as follows. The sample was obtained by holding 100 mg of Compound 1, crystalline Form I in in 1.5 mL of DCM by heating to 60° C. briefly in a sealed vial to dissolve most of the crystals, and then holding at 45° C. for a week. The resulting slurry was cooled to about 22° C. and analyzed by XRPD to confirm DCM solvate 2 was obtained, and it was then submitted for SCXRD analysis. The data shows the crystal consisted of only one major atropisomer at the biaryl rotation axis, and no solvents in the crystal lattice, due to the volatile nature of the DCM. Simulated XRPD powder pattern based on the solved structure was consistent with experimental XRPD pattern of Compound 1, crystalline Form II (FIG. 9), despite that they were obtained at different temperatures (100 K vs 22° C.).

During experiments to acquire SCXRD of Compound 1, BuOAc solvate 2, SCXRD of Compound 1, crystalline Form II was obtained as follows. A solution containing 22 mg of Compound 1 in 1.5 mL of BuOAc was seeded with seeds of Compound 1, crystalline Form II. The sample was held at about 50° C. for one week to form a suspension, which was cooled to about 22° C. and submitted for SCXRD analysis. Two different forms were obtained from the slurry, one being Compound 1, BuOAc solvate and the other being Compound 1, crystalline Form II, as shown in Table 2. The Compound 1, BuOAc solvate had 2 equivalents of BuOAc, and the Compound 1, crystalline Form II crystal contained 0.17 equivalents of water. Both forms consisted of only the major atropisomer at the biaryl rotation axis, as confirmed by ¹⁹F-NMR analysis of the dried solids (Compound 1, crystalline Form II). It was suspected that Compound 1, crystalline Form II crystals might be generated during the crystal growth period when the vial was held at 50° C.

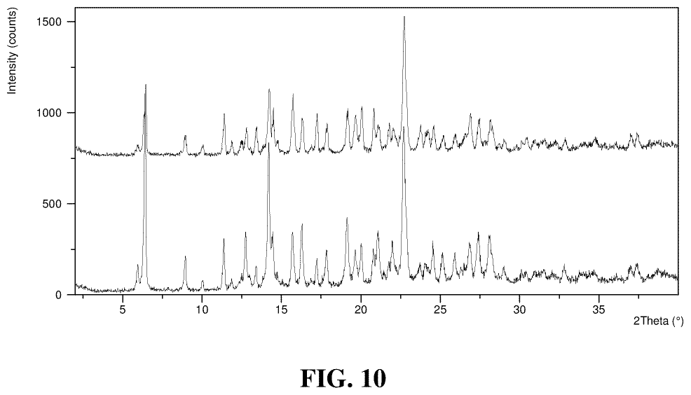

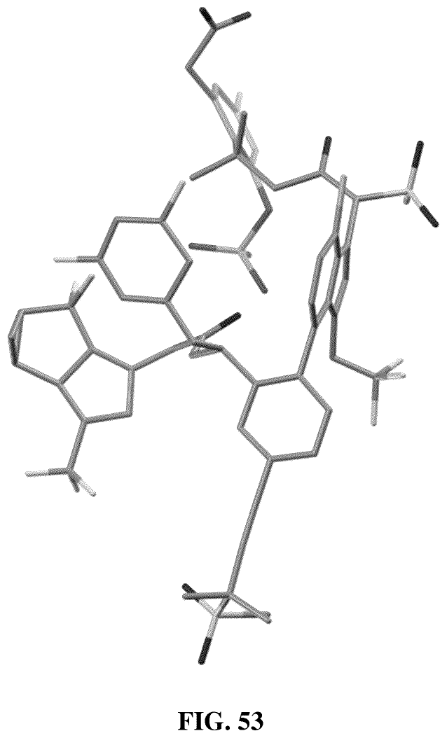

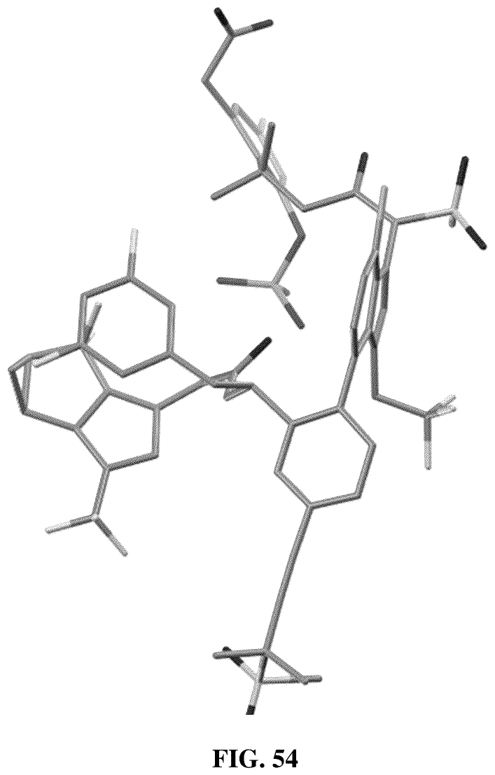

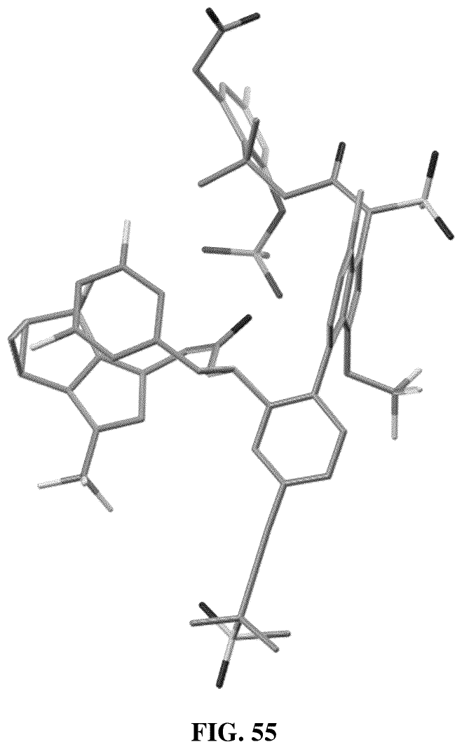

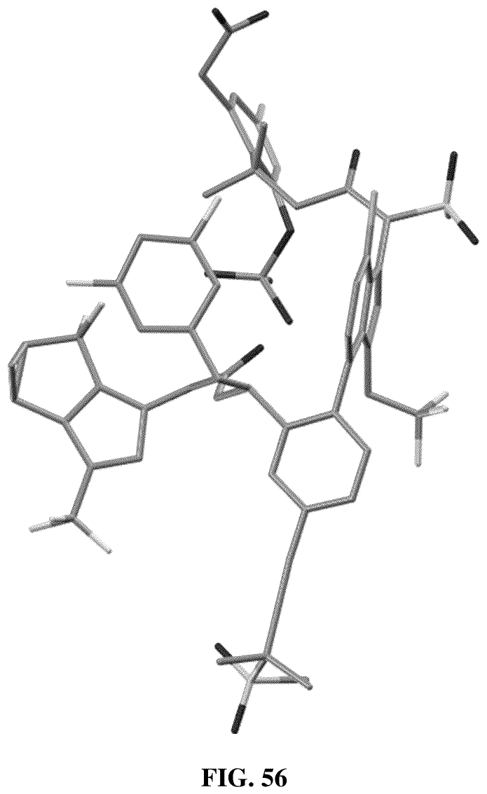

While Compound 1, crystalline Form II may be prepared from either DCM or BuOAc, the SCXRD data, provided in Table 2, showed slight differences in the parameters of the crystal lattices, and the presence of a trace amount of water in the Compound 1, crystalline Form II obtained from BuOAc (0.17 eq, or about 0.23% water). However, the peak positions of the XRPD patterns of both materials were similar (FIG. 10); therefore, these two were both designated as Compound 1, crystalline Form II. Molecular conformations of Compound 1, EtOAc Solvate 1, Compound 1, crystalline Form II from DCM, Compound 1, crystalline Form II from BuOAc, and Compound 1, BuOAc solvate 2 are shown in FIGS. 53-56.

TABLE 2

| | Forms | | | | | |
|---|---|---|---|---|---|---|
| | EtOAc | | Form II | | Form II | BuOAc |
| | Solvate 1 | | from DCM | | from BuOAc | Solvate 2 |
| Temp. | 100.0 | K | 100.0 | K | 100.15 | 100.15 |
| Crystal | Triclinic | | Monoclinic | | Monoclinic | Triclinic |
| system | | | | | | |
| Space | P1 | | P 1 21 1 | | P 1 21 1 | P1 |
| group | | | | | | |
| Unit cell | a = 14.0994(9) Å | | a = 14.0203(9) Å | | a = 14.0703(8) Å | a = 14.4034(7) Å |
| dimensions | b = 15.1390(11) Å | | b = 14.1202(7) Å | | b = 14.1382(8) Å | b = 15.2370(8) Å |
| | c = 17.4607(12) Å | | c = 15.1900(8) Å | | c = 15.2567(10) Å | c = 17.3968(9) Å |
| | α = 103.905(4)° | | α = 90° | | α = 90° | α = 102.924(3)° |
| | β = 100.607(4)° | | β = 101.243(3)° | | β = 101.110(3)° | β = 100.322(2)° |
| | γ = 90.101(4)° | | γ = 90° | | γ = 90° | γ = 90.060(2)° |
| Z | 2 | | 2 | | 2 | 2 |
| Density | 1.394 | g/cm3 | 1.460 | g/cm3 | 1.449 | 1.388 |

| | | | | |
|---|---|---|---|---|
| (calculated) | | | | |
| Goodness-of- | 1.215 | 1.057 | 1.065 | 1.032 |
| fit on F2 | | | | |
| Crystal | Compound | Compound 1 | Compound | Compound |
| composition | 1:EtOAc = | only | 1:water = | 1:BuOAc = |
| | 1:2.207 | | 1:0.17 | 1:2 |

A representative XRPD pattern of Compound 1, crystalline Form II (after drying the BuOAc solvate at 50° C. under vacuum) is shown in FIG. 11 and is characterized by sharp reflections, indicating crystallinity. An XRPD peak listing is provided in Table 3.

TABLE 3

| 2-Theta (°) | Relative Intensity (%) |
|---|---|
| 6.5 | 53 |
| 8.9 | 14 |
| 10.1 | 7 |
| 11.4 | 30 |
| 11.9 | 9 |
| 12.8 | 19 |
| 13.4 | 18 |
| 14.3 | 48 |
| 14.5 | 29 |
| 15.7 | 39 |
| 16.3 | 25 |
| 17.2 | 26 |
| 17.9 | 21 |
| 19.2 | 29 |
| 19.7 | 26 |
| 20.0 | 31 |
| 20.8 | 28 |
| 21.1 | 16 |
| 21.8 | 17 |
| 22.7 | 100 |
| 23.8 | 16 |
| 24.6 | 17 |
| 25.2 | 9 |
| 25.9 | 9 |
| 26.9 | 25 |
| 27.5 | 20 |
| 28.2 | 18 |
| 34.5 | 3 |
| 37.0 | 9 |

The DSC thermogram (FIG. 12) indicated an endotherm event with onset at about 201° C., attributed to melting. The TGA thermogram (FIG. 13) showed a weight loss of about 0.2% (from 25 to 150° C.), indicating that the solid is not solvated. DVS analysis (FIG. 14) indicated that the form is slightly hygroscopic with about 1% water uptake from 0 to 90% RH at 25° C.

### Example 3. Preparation of Compound 1, Crystalline Form III

About 50 mg of Compound 1, crystalline Form I was stirred in about 0.2 mL of IPAc at about 22° C. to form a solution. About 0.1 mL of n-butyl ether was charged as an antisolvent. The sample crystallized soon afterwards and was stirred over a period of 2 weeks. XRPD pattern (FIG. 15) of the wet cake showed it was a new solvate (comparing with Compound 1, IPAc Solvate 1), designated as Compound 1, IPAc solvate 2. The solid became Compound 1, crystalline Form III after drying at about 50° C. under vacuum.

Compound 1, crystalline Form III was also obtained after drying Compound 1, MIBK solvate 3 (FIG. 15) that had been obtained by stirring Compound 1 in a mixture of MIBK and DBE with the combined seeds of Compound 1, crystalline Forms I, II, III, and Compound 1, Material A for two days.

A representative XRPD pattern of Compound 1, crystalline Form III (after drying the IPAc solvate at 50° C. under vacuum) is shown in FIG. 16 and is characterized by sharp reflections, indicating crystallinity. An XRPD peak listing is provided in Table 4.

TABLE 4

| 2-Theta (°) | Relative Intensity (%) |
|---|---|
| 6.5 | 100 |
| 7.7 | 7 |
| 8.5 | 11 |
| 9.6 | 11 |
| 11.8 | 34 |
| 12.8 | 79 |
| 13.3 | 33 |
| 14.0 | 24 |
| 15.5 | 29 |
| 16.3 | 26 |
| 16.8 | 22 |
| 17.4 | 41 |
| 18.7 | 97 |
| 19.2 | 58 |
| 21.5 | 48 |
| 21.9 | 87 |
| 24.5 | 36 |
| 27.0 | 22 |
| 30.9 | 8 |
| 33.9 | 9 |
| 35.0 | 12 |

The DSC thermogram (FIG. 17) indicated an endotherm event with onset at about 175° C., attributed to melting. The TGA thermogram (FIG. 18) showed a weight loss of about 0.2% (from 25 to 150 C), indicating that the solid is not solvated. DVS analysis (FIG. 19) indicated that the form is slightly hygroscopic with about 1.6% water uptake from 0 to 90% RH at 25° C.

### Example 4. Preparation of Compound 1, Material A

During a salt screening experiment with sulfonic acid, a solution containing 0.1 g of Compound 1 in 0.2 mL of DCM was mixed with 1 eq. of sulfonic acid, charged with 0.05 mL of toluene, and stirred with a magnetic stir bar at about 22° C. During the 16-hour stirring period, a slurry formed. The slurry was centrifuged and XRPD analysis of the wet cake showed it was crystalline. After drying, the solid lost crystallinity and became Compound 1, Material A. Because Compound 1, Material A is a form of free base that was later made without sulfuric acid, it was determined that the wet cake was not a salt, but a DCM solvate of the free base (designated as Compound 1, DCM Solvate 1).

Compound 1, Material A was also obtained as follows. About 1 g of Compound 1, amorphous material was stirred in 1 mL of EtOAc to form a solution. It was seeded with Compound 1, crystalline Material A (prepared as described above), sonicated, and crystallized as a thick slurry within an hour. XRPD pattern of the wet cake (with a Kapton film cover to prevent solvent loss) is shown in FIG. 20. This wet form was designated as Compound 1, EtOAc Solvate 1. After drying, the solid lost crystallinity and became Compound 1, Material A.

Single crystals of Compound 1, EtOAc Solvate 1 were obtained as follows. A solution containing 30 mg of Compound 1 in 0.5 mL of EtOAc and 0.2 mL of heptanes was seeded with seeds of Compound 1, Material A. The sample was held at about 22° C. for a week to form a suspension, which was submitted for SCXRD analysis. The result showed 2.207 equivalents of EtOAc in the crystal, and confirmed that the crystal consisted of only the major atropisomer at the biaryl rotation axis.

Compound 1, Material A was also obtained after drying at 50 to 75° C. some solvates (as precursors) that had been obtained by stirring Compound 1 (preferably with the seeds of Material A) in the corresponding solvents with appropriate antisolvent such as diisopropyl ether, n-butyl ether, or heptanes, if necessary. The XRPD patterns of some of the solvate precursors of Compound 1, Material A are shown in FIG. 20.

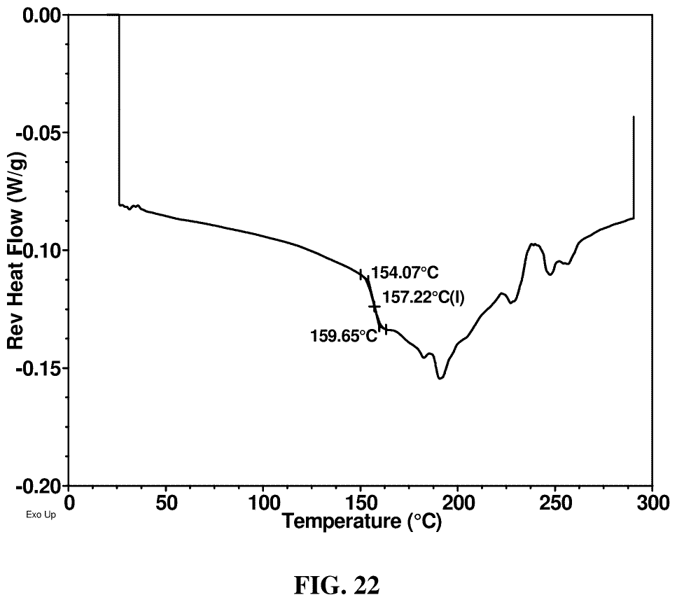

A representative XRPD pattern of Compound 1, Material A (after drying the EtOAc solvate at 50° C. under vacuum) is shown in FIG. 21 and is characterized by a halo almost identical with the amorphous material, indicating disordered material after drying. MDSC thermogram (FIG. 22) indicated a glass transition temperature at about 154° C. The TGA thermogram (FIG. 23) showed a weight loss of about 4.3% (from 25 to 150° C.) due to residual solvents as well as the hygroscopic nature of the solid. DVS analysis (FIG. 24) indicated that the form is hygroscopic with about 4.6% water uptake from 0 to 90% RH at 25° C.

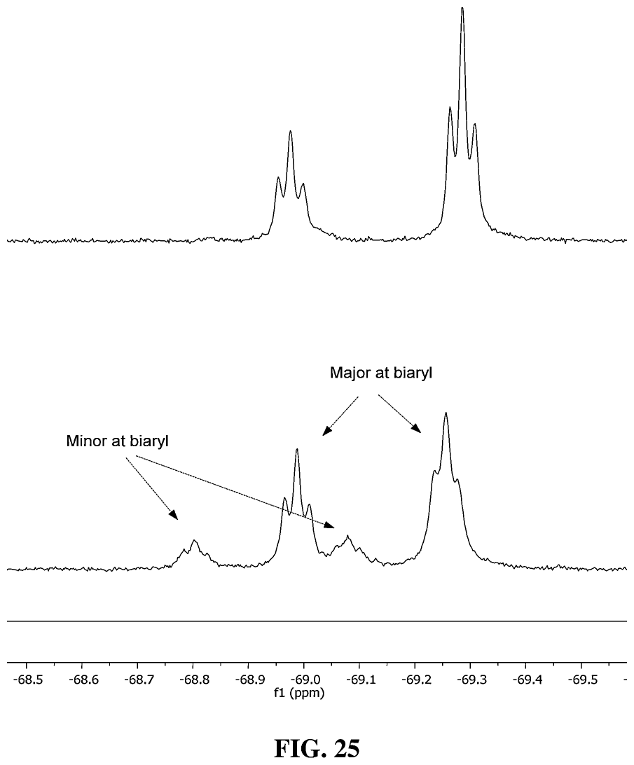

While the XRPD of Compound 1, Material A was similar to amorphous Compound 1, (FIG. 26), 19F-NMR analysis (FIG. 25) showed that Compound 1, Material A contains mostly the major atropisomers at the biaryl rotation axis, while amorphous Compound 1 showed four atropisomers.

### Example 5. Preparation of Compound 1, Amorphous Form

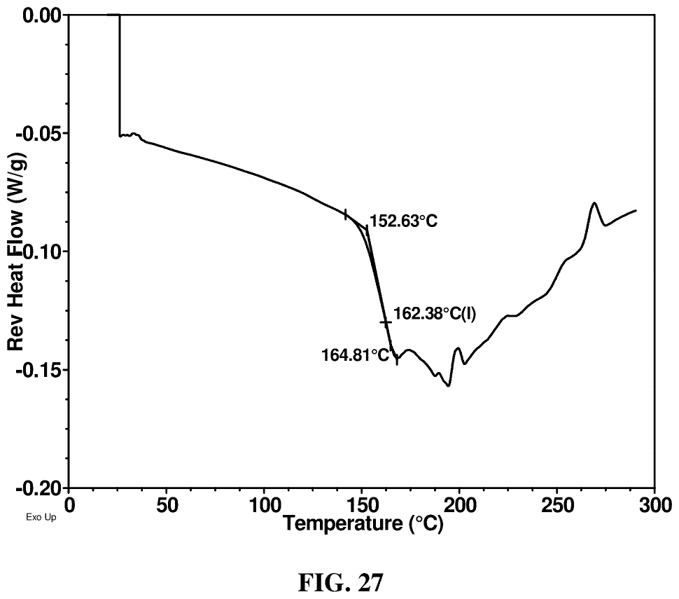

Amorphous Compound 1 was obtained by precipitation, drying solutions of Compound 1 in organic solvents (e.g., in a rotary evaporator), or lyophilizing aqueous solution of Compound 1. A representative XRPD pattern of amorphous Compound 1 is shown in FIG. 26 (after drying a solution of Compound 1 in acetone at 50° C. under vacuum). MDSC thermogram (FIG. 27) indicated a glass transition temperature at about 152° C. The TGA thermogram (FIG. 28) shows a weight loss of about 1.2% (from 25 to 150° C.), due to residual solvents as well as the hygroscopic nature of the solid.

### Example 6. Preparation of Compound 1 Maleate Co-Crystal

About 100 mg of Compound 1, crystalline Form I was stirred with 2 equivalents of maleic acid in 0.5 mL of MTBE at about 22° C. over a period of 20 hours. The sample was filtered and dried at 50° C. under vacuum. XRPD analysis showed a unique pattern, which was designated as Compound 1 maleate. Proton NMR showed about 1.15 equivalents of maleic acid.

A representative XRPD pattern of Compound 1 maleate (after drying at 50° C. under vacuum) is shown in FIG. 29 and is characterized by sharp reflections, indicating crystallinity. An XRPD peak listing is provided in Table 5.

TABLE 5

| 2-Theta (°) | Relative Intensity (%) |
|---|---|
| 6.0 | 24 |
| 6.7 | 38 |
| 9.0 | 16 |
| 10.4 | 28 |
| 12.3 | 62 |
| 13.4 | 86 |
| 13.7 | 29 |
| 15.0 | 13 |
| 16.1 | 92 |
| 17.2 | 60 |
| 18.0 | 59 |
| 19.2 | 24 |
| 20.1 | 47 |
| 21.0 | 40 |
| 21.6 | 99 |
| 21.8 | 100 |
| 22.4 | 95 |
| 23.4 | 14 |
| 24.2 | 17 |
| 24.8 | 68 |
| 25.5 | 37 |
| 26.3 | 17 |
| 27.0 | 21 |
| 27.7 | 26 |
| 28.4 | 20 |
| 29.5 | 27 |
| 30.2 | 20 |
| 30.8 | 22 |
| 32.5 | 19 |
| 33.8 | 8 |
| 35.3 | 13 |
| 35.8 | 14 |
| 37.4 | 11 |

The DSC thermogram (FIG. 30) of Compound 1 maleate indicated several endotherm events with onset at about 82° C., 124° C., and 197° C. The endotherm event with onset at 197° C. was attributed to melting of Compound 1, crystalline Form I (confirmed in another experiment that after heating to 180° C., a sample of Compound 1 maleate lost maleic acid and became Compound 1, crystalline Form I). The TGA thermogram (FIG. 31) showed a weight loss of about 0.25% from 25 to 75° C., and a further loss of about 9.7% from 75° C. to 175° C., attributed to loss of maleic acid (NMR analysis of a sample heated to 180° C. indicated residual maleic acid content of 0.01 equivalents). DVS analysis (FIG. 32) indicated that the form is hygroscopic with about 2.5% water uptake from 0 to 90% RH at 25° C.

### Example 7. Preparation of Compound 1, Succinate Co-Crystal

Compound 1 succinate was obtained as follows. About 100 mg of Compound 1, crystalline Form I was stirred with 1 equivalent of succinic acid in 1 mL of MTBE at about 22° C. over a period of 4 days. The sample was filtered and dried at 50° C. under vacuum. XRPD analysis showed a unique pattern, which was designated as Compound 1 succinate. Proton NMR shows about 0.83 equivalents of succinic acid.

A representative XRPD pattern of Compound 1 succinate (after drying at 50° C. under vacuum) is shown in FIG. 33 and is characterized by sharp reflections, indicating crystallinity. An XRPD peak listing is provided in Table 6.

TABLE 6

| 2-Theta | Relative Intensity (%) |
|---|---|
| 10.5 | 9 |
| 12.5 | 19 |
| 13.5 | 32 |
| 14.0 | 16 |
| 16.3 | 61 |
| 17.4 | 44 |
| 18.1 | 58 |
| 19.4 | 14 |
| 20.2 | 36 |
| 21.1 | 20 |
| 22.0 | 78 |
| 22.7 | 100 |
| 24.9 | 74 |
| 25.7 | 43 |
| 26.4 | 18 |
| 27.2 | 18 |
| 27.9 | 23 |
| 28.6 | 19 |
| 29.6 | 36 |
| 30.4 | 26 |
| 30.9 | 21 |
| 32.7 | 23 |
| 35.5 | 28 |
| 36.0 | 16 |
| 37.4 | 13 |
| 38.5 | 13 |

The DSC thermogram (FIG. 34) of Compound 1 succinate indicated several endotherm events with onset at about 39° C., 163° C., and 164° C. The TGA thermogram (FIG. 35) showed a weight loss of about 0.5% from 25 to 75° C., and a further loss of about 7.5% from 75° C. to 190° C. DVS analysis (FIG. 36) indicated that the form is slightly hygroscopic with about 1.7% water uptake from 0 to 90% RH at 25° C.

### Example 8. Preparation of Compound 2, Crystalline Form I

A mixture of amorphous Compound 2, diethyl ether, and dibutyl ether was stirred in a sealed vial at about 21° C. for a week, and remained as a clear solution with sticky transparent paste on the wall. The sample was left undisturbed without stirring for 8 months, and crystallized during this period. XRPD analysis of the solids confirmed that it was Compound 2, crystalline Form I. Since Compound 2 was observed to readily form solvates with organic solvents, and ethyl either was very volatile, it was believed that in solutions, it first formed an ethyl ether solvate, but lost the solvent during sample preparation.

This form was reproduced subsequently by stirring amorphous Compound 2 in ethyl ether, IPA, MTBE, or CPME to crystallize as solvates (precursors), and then drying at 40° C. to 70° C. XRPD patterns of some of solvate precursors of Compound 2, crystalline Form I are shown in FIG. 37.

A representative XRPD pattern of Compound 2, crystalline Form I (after drying the IPA solvate) is shown in FIG. 38 and is characterized by sharp reflections, indicating crystallinity. An XRPD peak listing is provided in Table 8.

TABLE 8

| 2-Theta (°) | Relative Intensity (%) |
|---|---|
| 6.0 | 22 |
| 6.5 | 23 |
| 8.3 | 18 |
| 8.9 | 25 |
| 9.4 | 24 |
| 9.9 | 19 |
| 11.1 | 27 |
| 11.9 | 51 |
| 13.1 | 26 |
| 13.6 | 40 |
| 14.1 | 32 |
| 15.3 | 51 |
| 16.0 | 17 |
| 17.2 | 66 |
| 17.9 | 60 |
| 18.2 | 74 |
| 18.9 | 100 |
| 19.5 | 89 |
| 20.0 | 51 |
| 20.5 | 27 |
| 21.1 | 59 |
| 21.8 | 40 |
| 22.9 | 49 |
| 23.3 | 44 |
| 24.0 | 34 |
| 26.8 | 11 |
| 27.8 | 14 |
| 29.6 | 23 |
| 32.7 | 11 |
| 33.6 | 24 |

The DSC thermogram (FIG. 39) of a sample prepared using IPA indicated multiple endothermic events with onset at 79° C., 115° C., 121° C., and 142° C. The TGA thermogram (FIG. 40) showed a weight loss of about 1.5% (from 25 to 112° C.), and significant weight loss after that due to decomposition. DVS analysis (FIG. 41) indicated that the form is hygroscopic with about 2.7% water uptake from 0 to 90% RH at 25° C.

### Example 10. Preparation of Compound 2, Form II

Solutions of Compound 2 in EtOH, acetone, MEK, DCM, THF, MeTHF, EtOAc, IPAc, or toluene were charged with antisolvent DBE and seeded with Compound 2, crystalline Form I solid. After stirring for about 1 day, slurries were formed and the solids were isolated by filtration, then dried under vacuum at about 40° C. XRPD analysis showed that the dried materials had similar patterns, which were designated as Compound 2, crystalline Form II. It is believed that Compound 2, crystalline Form II also went through various solvate forms. An example is shown in FIG. 42, in which the XRPD patterns of the solids (originally from acetone/heptanes) changed peak positions after drying under vacuum at 40° C.

To further investigate this, single crystals of Compound 2 acetone solvate was prepared by charging 0.5 mL of DBE into a solution of 0.1 g of Compound 2 in 0.5 mL acetone, followed by seeding of acetone solvate at about 21° C. The crystal structure parameters are shown in Table 9.

TABLE 9

| Temperature | 299.8 (5) K |
|---|---|
| Crystal system | Orthorhombic |
| Space group | P2₁2₁2₁ |
| Unit cell | a = 15.0553(5) Å |
| dimensions | b = 29.7410(10) Å |
| | c = 36.0634(10) Å |
| | α = 90° |
| | β = 90 |
| | γ = 90° |
| Z | 8 |
| Goodness-of-fit on F2 | 0.92 |

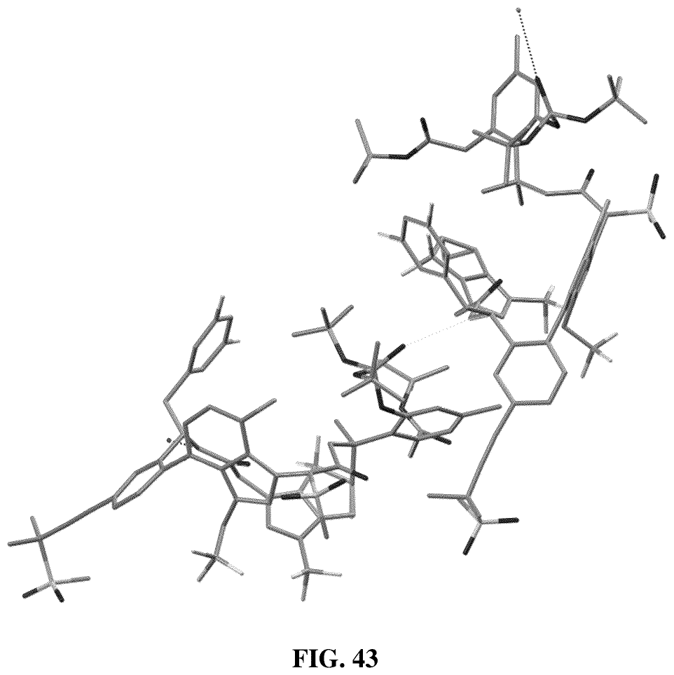

The single crystal structure of Compound 2 confirmed the molecular structure and absolute configuration. It was a solvated crystal form, composed of two Compound 2 molecules with disordered solvent in the asymmetric unit. The solvent was not modeled because of the disorder. Hydrogen bonding occurs from the amide NH moiety to the phosphate of an adjacent molecule, creating a one-dimensional hydrogen bond network along the c-axis, shown in FIG. 43.

A representative XRPD pattern of Compound 2, crystalline Form II (after drying the acetone solvate) is shown in FIG. 44 and is characterized by sharp reflections, indicating crystallinity. An XRPD peak listing is provided in Table 10.

TABLE 10

| 2-Theta (°) | Relative Intensity (%) |
|---|---|
| 6.0 | 36 |
| 6.5 | 22 |
| 7.0 | 26 |
| 7.7 | 17 |
| 8.2 | 33 |
| 8.7 | 42 |
| 9.8 | 23 |
| 11.1 | 44 |
| 11.8 | 35 |
| 13.0 | 45 |
| 14.0 | 33 |
| 14.5 | 38 |
| 15.0 | 78 |
| 15.8 | 29 |
| 16.9 | 62 |
| 17.7 | 48 |
| 18.1 | 38 |
| 18.7 | 30 |
| 19.0 | 41 |
| 19.3 | 68 |
| 19.8 | 42 |
| 20.2 | 65 |
| 20.8 | 57 |
| 21.3 | 100 |
| 22.6 | 51 |
| 23.1 | 50 |
| 23.6 | 55 |
| 24.6 | 13 |
| 27.8 | 17 |
| 33.1 | 18 |

The DSC thermogram (FIG. 45) indicated two endothermic events with onsets at about 119° C. and 130° C., respectively. The TGA thermogram (FIG. 46) showed a weight loss of about 1% (from 25 to 100° C.), and significant weight loss after that due to decomposition.

### Example 11. Preparation of Compound 2, Crystalline Form III

A solution of Compound 2 in MIBK was charged with antisolvent DBE and seeded with Compound 2, crystalline Form I solid. After stirring for about 1 day, a slurry was formed and the solid was isolated by filtration, then dried under vacuum at about 40° C. XRPD analysis showed that the dried material had unique pattern and was designated as Compound 2, crystalline Form III. It is believed to be a MIBK solvate, which lost most of its crystallinity upon drying at 70° C.

A representative XRPD pattern of Compound 2, crystalline Form III is shown in FIG. 47 and is characterized by sharp reflections, indicating crystallinity. An XRPD peak listing is provided in Table 11.

TABLE 11

| 2-Theta (°) | Relative Intensity (%) |
|---|---|
| 6.4 | 37 |
| 7.5 | 26 |
| 8.7 | 19 |
| 10.8 | 19 |
| 12.2 | 29 |
| 12.7 | 11 |
| 13.4 | 44 |
| 15.0 | 77 |
| 15.9 | 21 |
| 16.3 | 17 |
| 17.0 | 33 |
| 18.0 | 36 |
| 18.5 | 12 |
| 18.9 | 32 |
| 19.2 | 35 |
| 20.1 | 78 |
| 20.6 | 23 |
| 21.0 | 57 |
| 21.8 | 100 |
| 22.1 | 61 |
| 22.5 | 21 |
| 23.5 | 23 |
| 24.7 | 25 |
| 26.4 | 18 |
| 27.0 | 16 |
| 28.2 | 15 |
| 30.2 | 21 |
| 32.6 | 27 |
| 36.9 | 9 |

The DSC thermogram (FIG. 48) indicated multiple endothermic events with onsets at about 109° C., 126° C., and 141° C. The TGA thermogram (FIG. 49) showed a weight loss of about 7% (from 25 to 100° C., due to loss of MIBK), and significant weight loss after that due to decomposition. 5

### Example 12. Preparation of Compound 2, Amorphous Form

Amorphous Compound 2 was prepared by charging a concentrated solution of Compound 2 in MTBE into n-heptane to form a slurry at about 21° C., filter, wash with n-heptane, and drying at about 30° C. under nitrogen sweep. XRPD pattern of the solid is shown in FIG. 50. DSC thermogram of the sample is shown in FIG. 51.

### Example 13. Preparation of Compound 1, Oxalate Co-Crystal

About 100 mg of Compound 1, crystalline Form I was stirred with 1 equivalent of oxalic acid in 1 mL of MTBE at about 22° C. over a period of 13 days. The sample was filtered and dried at 50° C. under vacuum. XRPD analysis showed a unique pattern, which was designated as Compound 1 oxalate.

A representative XRPD pattern of Compound 1 oxalate (after drying at 50° C. under vacuum) is shown in FIG. 57. An XRPD peak listing is provided in Table 12.

TABLE 12

| 2-Theta (°) | Relative Intensity (%) |
|---|---|
| 6.0 | 11 |
| 8.2 | 5 |
| 10.6 | 5 |
| 12.7 | 13 |
| 13.5 | 28 |
| 14.4 | 38 |
| 15.2 | 18 |
| 15.7 | 37 |
| 17.1 | 46 |
| 17.4 | 38 |
| 19.1 | 19 |
| 19.7 | 45 |
| 20.3 | 40 |
| 21.9 | 100 |
| 22.9 | 41 |
| 23.7 | 23 |
| 24.4 | 64 |
| 25.8 | 22 |
| 26.7 | 49 |
| 28.3 | 27 |
| 28.9 | 21 |
| 30.3 | 11 |
| 32.2 | 14 |
| 34.8 | 10 |
| 36.4 | 7 |
| 37.2 | 10 |

The DSC thermogram (FIG. 58) of Compound 1 oxalate indicated several endotherm events with onset at about 82° C., 165° C., and 187° C.

### Example 14. Preparation of Compound 1, Gentisate Co-Crystal

About 100 mg of Compound 1, crystalline Form II was stirred with 1 equivalent of gentisic acid in 1 mL of MTBE at about 22° C. over a period of 13 days. The sample remained as sticky substance. It was dried in the vacuum oven and switched to 0.25 mL of MeCN. After stirring overnight, the sample was dried at 50° C. under vacuum and then charged with 0.5 mL of MTBE. After stirring overnight, the sample was dried again at 50° C. under vacuum and then switched to 0.4 mL of EtOAc. After 5 days, the sample was again dried at 50° C. under vacuum, and charged with 0.5 mL of MTBE. After stirring for 13 days at about 22° C., the sample was filtered and dried at 50° C. under vacuum. XRPD analysis showed a unique pattern, which was designated as Compound 1 gentisate.

A representative XRPD pattern of Compound 1 gentisate (after drying at 50° C. under vacuum) is shown in FIG. 59. An XRPD peak listing is provided in Table 13.

TABLE 13

| 2-Theta (°) | Relative Intensity (%) |
|---|---|
| 5.3 | 100 |
| 5.9 | 18 |
| 8.2 | 9 |
| 10.6 | 8 |
| 12.2 | 10 |
| 12.7 | 20 |
| 13.5 | 11 |
| 14.3 | 17 |
| 15.0 | 11 |
| 15.8 | 31 |
| 16.9 | 43 |
| 18.5 | 9 |
| 18.8 | 11 |
| 20.2 | 18 |
| 20.8 | 10 |
| 21.5 | 14 |
| 21.7 | 37 |
| 22.9 | 4 |
| 24.0 | 8 |
| 24.4 | 9 |
| 26.0 | 12 |
| 26.7 | 17 |
| 28.2 | 5 |
| 28.8 | 8 |
| 29.7 | 7 |

The DSC thermogram (FIG. 60) of Compound 1 gentisate indicated several endotherm events with onset at about 77° C. and 120° C.

### Example 15. Preparation of Compound 1, L-Tartrate Co-Crystal

About 100 mg of Compound 1, crystalline Form I was stirred with 1 equivalent of L-tartaric acid in 1 mL of MTBE at about 22° C. over a period of 17 days. The sample was dried at 50° C. under vacuum and then charged with 0.3 mL of EtOAc and seeds of Material A. After stirring for 1 day, the sample was again dried at 50° C. under vacuum, and charged with 0.5 mL of MTBE. After stirring for 28 days at about 22° C., the sample was filtered and dried at 50° C. under vacuum. XRPD analysis showed a unique pattern, which was designated as Compound 1, L-tartrate.

A representative XRPD pattern of Compound 1, L-tartrate (after drying at 50° C. under vacuum) is shown in FIG. 61. An XRPD peak listing is provided in Table 14.

TABLE 14

| 2-Theta (°) | Relative Intensity (%) |
|---|---|
| 5.3 | 51 |
| 5.9 | 25 |
| 8.2 | 15 |
| 10.6 | 13 |
| 12.7 | 31 |
| 13.5 | 37 |
| 14.3 | 32 |
| 15.1 | 31 |
| 15.7 | 42 |
| 17.0 | 88 |
| 18.9 | 45 |
| 20.2 | 92 |
| 20.8 | 72 |
| 21.8 | 100 |
| 22.8 | 28 |
| 24.5 | 39 |
| 26.0 | 23 |
| 26.7 | 48 |
| 29.8 | 47 |
| 32.1 | 37 |
| 36.9 | 33 |

The DSC thermogram (FIG. 62) of Compound 1, L-tartrate indicated two endotherm events with onset at about 109° C. and 162° C.

### Example 16. Preparation of Compound 1, Vanillate Co-Crystal

About 100 mg of Compound 1, crystalline Form I was stirred with 1 equivalent of vanillic acid in 1 mL of MTBE at about 22° C. over a period of 17 days. The sample was dried at 50° C. under vacuum and then charged with 0.3 mL of EtOAc and seeded with seeds of Material A. After stirring for 1 days, the sample was again dried at 50° C. under vacuum, and charged with 0.5 mL of MTBE. After stirring for 28 days at about 22° C., the sample was filtered and dried at 50° C. under vacuum. XRPD analysis showed a unique pattern, which was designated as Compound 1 vanillate.

A representative XRPD pattern of Compound 1 vanillate (after drying at 50° C. under vacuum) is shown in FIG. 63. An XRPD peak listing is provided in Table 15.

TABLE 15

| 2-Theta (°) | Relative Intensity (%) |
|---|---|
| 5.2 | 69 |
| 5.9 | 16 |
| 8.2 | 23 |
| 10.6 | 14 |
| 12.2 | 18 |
| 12.7 | 44 |
| 13.5 | 41 |
| 14.4 | 41 |
| 15.1 | 37 |
| 15.7 | 42 |
| 17.0 | 82 |
| 18.5 | 21 |
| 18.9 | 27 |
| 19.5 | 15 |
| 20.1 | 57 |
| 21.5 | 72 |
| 21.7 | 100 |
| 22.8 | 18 |
| 23.6 | 12 |
| 24.0 | 23 |
| 24.3 | 34 |
| 24.6 | 37 |
| 26.0 | 26 |
| 26.7 | 38 |
| 27.1 | 31 |
| 28.2 | 17 |
| 28.8 | 21 |
| 29.7 | 23 |
| 30.2 | 14 |
| 36.3 | 13 |

The DSC thermogram (FIG. 64) of Compound 1 vanillate indicated several endotherm events with onset at about 102° C., 132° C., and 184° C.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

1. A crystalline form of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid.

2. The crystalline form of claim 1, which is selected from crystalline Form I, crystalline Form II, and crystalline Form III.

3. The crystalline form of claim 1, which is crystalline Form I.

4. The crystalline form of claim 3, wherein the crystalline Form I has at least three XRPD peaks, in terms of 2-theta±0.2°, selected from 6.2°, 6.6°, 8.7°, 10.5°, 12.4°, 12.6°, 13.8°, 23.1°, and 25.7°.

5. The crystalline form of claim 3, wherein the crystalline Form I is characterized by an XRPD pattern substantially as shown in FIG. 4.

6. The crystalline form of claim 3, wherein the crystalline Form I is characterized by a DSC thermogram having a melting onset at about 202° C.

7. The crystalline form of claim 3, wherein the crystalline Form I is characterized by a DSC thermogram substantially as shown in FIG. 5.

8. The crystalline form of claim 1, which is crystalline Form II.

9-17. (canceled)

18. A solvate form of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid.

19. The solvate form of claim 18, which is selected from an acetonitrile solvate form, a butyronitrile solvate, a methyl ethyl ketone solvate form, a methyl isobutyl ketone solvate form, a toluene solvate form, a nitromethane solvate form, a diethyl ether solvate form, a cyclopentyl methyl ether solvate form, an anisole solvate form, a xylene solvate form, a dichloromethane solvate form, a chloroform solvate form, an acetophenone solvate form, a dichloroethane solvate form, a methyl acetate solvate form, an ethylacetate solvate form, an isopropyl acetate solvate form, a n-butyl acetate solvate form, and a cyclohexane solvate form.

20. A co-crystal of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid.

21. The co-crystal of claim 20, which is selected from the group consisting of:
a 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid maleic acid co-crystal;
a 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid succinic acid co-crystal;
a 2-(2-(4-(N-(4-chloro-7-(2-((S)-1-(2-((3bS,4aR)-5,5-difluoro-3-(trifluoromethyl)-3b, 4,4a, 5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid oxalic acid co-crystal;
a 2-(2-(4-(N-(4-chloro-7-(2-((S)-1-(2-((3bS,4aR)-5,5-difluoro-3-(trifluoromethyl)-3b,4,4a, 5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid gentisic acid co-crystal;
a 2-(2-(4-(N-(4-chloro-7-(2-((S)-1-(2-((3bS,4aR)-5,5-difluoro-3-(trifluoromethyl)-3b,4,4a, 5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid L-tartaric acid co-crystal; and
a 2-(2-(4-(N-(4-chloro-7-(2-((S)-1-(2-((3bS,4aR)-5,5-difluoro-3-(trifluoromethyl)-3b,4,4a, 5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid vanillic acid co-crystal.

22-50. (canceled)

51. A pharmaceutical composition comprising a therapeutically effective amount of a crystalline form of claim 1, and a pharmaceutically acceptable excipient.

52. The pharmaceutical composition of claim 51, further comprising one, two, three, or four additional therapeutic agents.

53. The pharmaceutical composition of claim 52, wherein the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

54-55. (canceled)

56. A method of treating or preventing a human immunodeficiency virus (HIV) infection in a patient in need thereof comprising administering to the patient a therapeutically effective amount of a crystalline form of claim 1.

57. A method of treating a human immunodeficiency virus (HIV) infection in a heavily treatment-experienced patient, the method comprising administering to the patient a therapeutically effective amount of a crystalline form of claim 1.

58. The method of claim 56 or 57, wherein the method further comprises administering a therapeutically effective amount of one, two, three, or four additional therapeutic agents, or a pharmaceutically acceptable salt thereof.

59. The method of claim 58, wherein the one, two, three, or four additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

60-70. (canceled)
